(19) United States
(12) Patent Application Publication
(10) Pub. No.: US 2011/0020272 A1
(43) Pub. Date: Jan. 27, 2011

(54) COMBINATION THERAPY FOR TREATING HEPATITIS VIRAL INFECTION

(76) Inventor: Ulrich SCHUBERT, Jena (DE)

Correspondence Address: WILMERHALE/BOSTON, 60 STATE STREET, BOSTON, MA, 02109, US

(21) Appl. No.: 12/843,650

(22) Filed: Jul. 26, 2010

Related U.S. Application Data
(60) Provisional application No. 61/296,363, filed on Jan. 19, 2010.

(30) Foreign Application Priority Data
Jul. 24, 2009 (DE) 102009028015.4
Jan. 19, 2010 (EP) 10151135.0

(51) Int. Cl. A61K 38/20; A61K 38/21; A61K 31/56; A61K 38/45; A61K 38/16; A61K 31/7056; A61K 31/708; A61K 31/353; A61K 31/407; A61K 31/426; A61P 31/14

(52) U.S. Cl. 424/85.2; 424/85.4; 514/171; 424/94.5; 514/4.3; 424/85.5; 424/85.6; 424/85.7; 514/43; 514/46; 514/456; 514/421; 514/371

(57) ABSTRACT

Disclosed are methods of using proteasome inhibitors (PI) in combinations with other pharmaceutically active agents for treating viral hepatitis infections, for example, for treating therapy-resistant and -refractory viral hepatitis infections. Also disclosed are pharmaceutical compositions and kits of pharmaceutical compositions which can be used for treating viral hepatitis infections, for example, for treating therapy-resistant and refractory viral hepatitis infections.

### REFERENCE TO RELATED APPLICATIONS

This application claims priority to German Patent Application No. DE 10 2009 028 015.4, filed Jul. 24, 2009, European Patent Application No. EP 10 151 135.0, filed Jan. 19, 2010, and to U.S. Provisional Application No. 61/296,363, filed Jan. 19, 2010, all of which are incorporated by reference herein in their entirety.

### FIELD OF THE INVENTION

The invention relates generally to the use of proteasome inhibitors in combination with other pharmaceutically active agents for treating virally-induced hepatitis, and more specifically to use of same for treating hepatitis induced by HCV infection.

### BACKGROUND OF THE INVENTION

Pegylated interferon (PEG-IFN) alpha-2a or alpha-2b in combination with nucleoside analog ribavirin when being administered for 24 or 48 weeks currently is the standard therapy for patients suffering from chronic HCV infections (see Practice Guidelines: Diagnosis, Management and Treatment of Hepatitis C, American Association for the Study of Liver Disease AASLD; e.g., Hepatology 2009, 48:1335-74). The aim of the standard therapy is to achieve elimination of the HC virus, meaning that no HCV-RNA is detectable after treatment. The guanosine analog ribavirin in combination with interferons has been authorized for therapy of chronic HCV infections since 1999. However, the mode of action of this medicament is only partially understood. A complete elimination of HCV after administration of Ribavirin without IFN is not to be expected.

The standard therapy of IFN and Ribavirin is frequently associated with side effects which can be partially attributed to the specific substance respectively. The most frequently observed side effects of an IFN therapy are flu-like symptoms such as fever, headache, muscle pain, joint pain as well as fatigue, loss of appetite and loss of weight. Moreover, neuropsychiatric side effects including mood swings, insomnia, anxiety, depression, psychosis, suicidal ideation, actual suicide and homicide have been described. Pegylated interferon may also induce autoimmune disorders, or may worsen preexisting autoimmune disorders, e.g., autoimmune thyroiditis. A frequent side effect observed with ribavirin treatment is anemia, particularly hemolytic anemia, which necessitates continuous control of blood parameters during therapy. Other ribavirin-associated adverse events include mild lymphopenia, hyperuricemia, itching, rash, cough, and nasal stuffiness.

Therapy related adverse events are a major reason for patients discontinuing or outwardly refusing therapy. Quite a number of patients are not even eligible for the standard therapy, due to, e.g., preexisting conditions rendering an adverse event potentially fatal.

The above described adverse events are, at least partially, related to IFN and/or ribavirin exposure. Reducing such exposure by means of novel or improved methods or agents of treatment is, hence, desirable.

In principle, patients undergoing treatment for viral hepatitis, and particularly for hepatitis C, can be categorized as a result of the treatment success “responder”, “non-responder” and “relapser”. A response is typically understood to refer to a sustained decrease of the virus load below the detection limit for at least 6 months after standard therapy has ceased (“sustained virological response”, SVR). A relapse typically refers to a complete virological response up until the 24th week of treatment at the latest. However, after the standard therapy has ceased, a renewed increase of virus load may be observed (therapy refractory). Non-responders are patients for which typically no decrease of virus load by a factor of least 2 log steps is observed during 24 weeks or for which up to week 24, HCV-RNA are still detectable during standard therapy (therapy resistant).

For example, more than 50% of patients infected with HCV of Genotype 1 do not react towards standard therapy (“non-responder”) or suffer from a setback after therapy has ended (“relapser”). Certain patient groups are also less likely to experience a benign course of the disease; e.g., non-African-American origin was shown to be a characteristic associated with a favorable treatment response in HCV-infected individuals in at least one clinical trial. Treatment options, especially for non-responders and relapsed patients, are sparse (Kronenberger, B., Zeuzem, S., Annals of Hepatology 2009; 8: 103).

Evidently, a considerable medical need exists to develop new or improved treatment options for the treatment of hepatitis caused by viral infection, and specifically for Hepatitis C. As a precautionary note, titles and headings in as far as they appear herein are for convenient reference only, and shall in no way affect the interpretation of the present disclosure.

### BRIEF DESCRIPTION OF THE DRAWINGS

FIG. 1 is a graphic representation of the demographics of the clinical trial. 9 patients selected to be refractory to standard therapy or relapsed after standard therapy were treated with 1.3 μg/m² body surface of bortezomib 4 times over the course of 11 days. Thereafter, 6 of these 9 patients decided to voluntarily reenter standard therapy (Re-Therapy). 1 of these six patients prematurely terminated the study, 5 other patients continued to week 24 after termination of bortezomib treatment.

FIG. 2 is a graphic representation of the development of HCV RNA levels measured in patient blood samples over the course of the study. HCV RNA levels decreased in all patients from Visit 8 to Visit 11 and remained low until Visit 15, where all but one patient had HCV RNA levels below 10 IU/mL. “Start Re-Therapy” refers to the point in time of re-uptake of standard therapy by patients having agreed to re-enter therapy with interferon/ribavirin.

FIG. 3 is a graphic representation of the genetic make-up of the model HCV replicon system.

FIG. 4 is a graphic representation of luciferase activities in lysates of cells treated with S-2209, Interferon alpha (IFN-a) and/or Ribavirin (RbV) compared to untreated controls after treatment according to six different treatment protocols (protocols I to VI). Briefly, protocol I involved treatment of HCV infected Huh-7 cells with proteasome inhibitor followed by IFN-a/Rbv (protocol I), IFN-a/Rbv followed by proteasome inhibitor (protocol II), proteasome inhibitor followed by a treatment free period followed by IFN-a/Rbv treatment (protocol III), or a single, double or triple treatment with a combination of all three substances at 24 h intervals (protocols IV, V and VI, respectively).

### SUMMARY OF THE INVENTION

In one aspect, the invention provides a kit for the treatment of a hepatitis viral infection in a human or animal who does not respond or is refractory to treatment with at least one pharmaceutically active agent for treatment of a viral hepatitis infection, the kit comprising: (a) first pharmaceutical container containing a composition comprising an effective amount of a proteasome inhibitor; (b) a second pharmaceutical container containing a composition comprising an effective amount of a first pharmaceutically active agent for treatment of a viral hepatitis infection, wherein the first pharmaceutically active agent for treatment of a viral hepatitis infection is an agent which supports or assists a human or animal body's natural response in dealing with a viral infection or interferes with the function of a viral target; and (c) instructions in paper or electronic form advising a user to apply the kit for the treatment of a human or animal individual who does not respond or is refractory to treatment with at least one pharmaceutically active agent for treatment of a viral hepatitis infection.

In some embodiments, the second container further contains a composition comprising an effective amount of a second pharmaceutically active agent such that one of the first or the second pharmaceutically active agent assists a human or animal body's natural response in dealing with a viral infection, while the other of the first or second pharmaceutically active agents interferes with the function of a viral target, wherein the first and the second pharmaceutically active agent are both different from the proteasome inhibitor.

In some embodiments, the kit further comprises third pharmaceutical container containing a composition comprising an effective amount of a second pharmaceutically active agent such that one of the first or the second pharmaceutically active agent assists a human or animal body's natural response in dealing with a viral infection, while the other of the first or second pharmaceutically active agents interferes with the function of a viral target, wherein the first and the second pharmaceutically active agent are both different from the proteasome inhibitor.

In one aspect, the invention provides a method for treating a hepatitis viral infection in a human or animal individual who does not respond or is refractory to treatment with a pharmaceutically active agent for treatment of viral hepatitis infection, the method comprising administering to the human or animal in need thereof an effective amount of a proteasome inhibitor and an effective amount of a first pharmaceutically active agent for treatment of a viral hepatitis infection, wherein the first pharmaceutically active agent for treatment of a viral hepatitis infection is an agent which supports or assists a human or animal body's natural response in dealing with a viral infection or interferes with the function of a viral target.

In some embodiments, the method further comprises administering to the human or animal an effective amount of a second pharmaceutically active agent for treatment of a viral hepatitis infection such that one of the first or the second pharmaceutically active agent assists a human or animal body's natural response in dealing with a viral infection, while the other of the first or second pharmaceutically active agents interferes with the function of a viral target, wherein the first and the second pharmaceutically active agent are both different from the proteasome inhibitor.

In one aspect, the invention provides a pharmaceutical composition comprising an effective amount of a proteasome inhibitor and an effective amount of a first pharmaceutically active agent for treatment of a viral hepatitis infection, wherein the first pharmaceutically active agent for treatment of a viral hepatitis infection is an agent which supports or assists a human or animal body's natural response in dealing with a viral infection or interferes with the function of a viral target.

In some embodiments, the pharmaceutical composition further comprises an effective amount of a second pharmaceutically active agent for treatment of a viral hepatitis infection such that one of the first or the second pharmaceutically active agent assists a human or animal body's natural response in dealing with a viral infection, while the other of the first or second pharmaceutically active agents interferes with the function of a viral target, wherein the first and the second pharmaceutically active agent are both different from the proteasome inhibitor.

In one aspect, the invention provides a kit for the treatment of a hepatitis viral infection in a human or animal who does not respond or is refractory to treatment with a pharmaceutically active agent for treatment of a viral hepatitis infection, the kit comprising: a first pharmaceutical container containing a composition comprising an effective amount of a proteasome inhibitor; a second pharmaceutical container containing a composition comprising an effective amount of a first pharmaceutically active agent for treatment of a viral hepatitis infection; and instructions in paper or electronic form advising a user to apply the kit for the treatment of a human or animal individual who does not respond or is refractory to treatment with at least one pharmaceutically active agent for treatment of a viral hepatitis infection.

In some embodiments, the second container further contains a composition comprising an effective amount of a second pharmaceutically active agent for treatment of a viral hepatitis infection which is different from the first pharmaceutically active agent for treatment of a viral hepatitis infection.

In some embodiments, the kit further comprises a third pharmaceutical container containing a composition comprising an effective amount of a second pharmaceutically active agent for treatment of a viral hepatitis infection which is different from the first pharmaceutically active agent for treatment of a viral hepatitis infection.

In one aspect, the invention provides a kit for the treatment of a hepatitis viral infection in a human or animal who does not respond or is refractory to treatment with a pharmaceutically active agent for treatment of a viral hepatitis infection, the kit comprising: a first pharmaceutical container containing a composition comprising an effective amount of a proteasome inhibitor; a third pharmaceutical container containing a composition comprising an effective amount of a second pharmaceutically active agent for treatment of a viral hepatitis infection; and instructions in paper or electronic form advising a user to apply the kit for the treatment of a human or animal individual who does not respond or is refractory to treatment with at least one pharmaceutically active agent for treatment of a viral hepatitis infection.

In some embodiments, the third container further contains a composition comprising an effective amount of a first pharmaceutically active agent for treatment of a viral hepatitis infection which is different from the first pharmaceutically active agent for treatment of a viral hepatitis infection.

In some embodiments, the kit further comprises a second pharmaceutical container containing a composition comprising an effective amount of a first pharmaceutically active agent for treatment of a viral hepatitis infection which is different from the second pharmaceutically active agent for treatment of a viral hepatitis infection.

In some embodiments, the proteasome inhibitor is a proteasome-specific inhibitor; the first pharmaceutically active agent for treatment of a viral hepatitis infection is an agent which supports or assists a human or animal body's natural response in dealing with a viral infection; and the second pharmaceutically active agent for treatment of a viral hepatitis infection is an agent which interferes with the function of a viral target.

In some embodiments, the proteasome inhibitor is a proteasome-specific inhibitor; the first pharmaceutically active agent for treatment of a viral hepatitis infection is an interferon or a derivative thereof, an interleukin, a steroid, an immunomodulator, an immunosuppressant or an inhibitor of assisted protein folding; and the second pharmaceutically active agent for treatment of a viral hepatitis infection is an inhibitor of HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the protease inhibitor is a protease-specific inhibitor; the first pharmaceutically active agent for treatment of a viral hepatitis infection is an interferon or a derivative thereof; and the second pharmaceutically active agent for treatment of a viral hepatitis infection is a nucleoside analog or a nucleotide analog.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040, or a glycosylated, pegylated or hesylated form thereof; and the second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

In some embodiments, the first pharmaceutically active agent for treatment of a viral hepatitis infection is a pegylated form of interferon-alpha, beta, gamma or omega; and the second pharmaceutically active agent for treatment of a viral hepatitis infection is ribavirin. In some embodiments, the proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins (−)-epigallocatechin-3-gallate, Disulfuram, acridine derivatives, tetra-acridine derivatives with betulinic acid, as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides, TMC-95A, TMC-95A endocyclic oxindole-phenyl clamp (BIA-1a) derivatives, TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S, 4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

In some embodiments, the proteasome inhibitor is PS-273, PS-341, PS-519 or S-2209.

In some embodiments, the proteasome inhibitor is PS-341, the first pharmaceutically active agent for treatment of a viral hepatitis infection is pegylated interferon alpha, and the second pharmaceutically active agent for treatment of a viral hepatitis infection is ribavirin.

In some embodiments, the proteasome inhibitor is S-2209.

In some embodiments, the proteasome inhibitor is S-2209, the first pharmaceutically active agent for treatment of a viral hepatitis infection is pegylated interferon alpha, and the second pharmaceutically active agent for treatment of a viral hepatitis infection is ribavirin.

In some embodiments, the instructions advise the user to administer the compositions of the first and the second pharmaceutical containers concurrently.

In some embodiments, the instructions advise the user to first administer the composition of the first pharmaceutical container and administer the composition of the second pharmaceutical container subsequently after a delay.

In some embodiments, the instructions advise the user to first administer the composition of the second pharmaceutical container and administer the composition of the first pharmaceutical container subsequently after a delay.

In some embodiments, the instructions further advise the user of the length of the delay. In some embodiments, the delay is from about 2 weeks to about 8 weeks.

In some embodiments, the instructions advise the user to perform one to two rounds of administrations, each round consisting of 3 to 10 administrations of the composition of the first pharmaceutical container.

In some embodiments, the instructions advise the user to administer the first pharmaceutically active agent for treatment of a viral hepatitis infection for not more than about 50% of the duration otherwise recommended for the treatment of viral hepatitis infection with the first pharmaceutically active agent and/or to administer not more than about 66% of the dose recommended for the treatment of hepatitis viral infection with the first pharmaceutically active agent for treatment of a viral hepatitis infection.

In some embodiments, the instructions advise the user to apply the kit for the treatment of a human or animal individual ineligible for or unwilling to undergo treatment with a pharmaceutically active agent for treatment of a viral hepatitis infection.

In some embodiments, the hepatitis viral infection is an infection with Hepatitis C virus.

In another aspect, the invention provides a method for treating a hepatitis viral infection in a human or animal individual who does not respond or is refractory to treatment with a pharmaceutically active agent for treatment of viral hepatitis infection, the method comprising administering to the human or animal in need thereof an effective amount of a proteasome inhibitor and an effective amount of a first pharmaceutically active agent for treatment of a viral hepatitis infection.

In some embodiments, the method further comprises administering to the human or animal an effective amount of a second pharmaceutically active agent for treatment of a viral hepatitis infection which is different from the first pharmaceutically active agent.

In some embodiments, the proteasome inhibitor is a proteasome-specific inhibitor; and the first pharmaceutically active agent for treatment of a viral hepatitis infection is an agent which supports or assists a human or animal body's natural response to a viral infection or an agent that interferes with the function of a viral target.

In another aspect, the invention provides a method for treating a hepatitis viral infection in a human or animal individual who does not respond or is refractory to treatment with a pharmaceutically active agent for treatment of viral hepatitis infection, the method comprising administering to the human or animal in need thereof an effective amount of a proteasome inhibitor and an effective amount of a second pharmaceutically active agent for treatment of a viral hepatitis infection.

In some embodiments, the method further comprises administering to the human or animal an effective amount of a first pharmaceutically active agent for treatment of a viral hepatitis infection which is different from the second pharmaceutically active agent.

In some embodiments, the proteasome inhibitor is a proteasome-specific inhibitor; the first pharmaceutically active agent for treatment of a viral hepatitis infection is an agent which support or assists the human or animal body's natural response to a viral infection; and the second pharmaceutically active agent for treatment of a viral hepatitis infection is an agent that interferes with the function of a viral target.

In some embodiments, the proteasome inhibitor is a proteasome-specific inhibitor; and the first pharmaceutically active agent for treatment of a viral hepatitis infection is an interferon or a derivative thereof, an interleukin, a steroid, an immunomodulator, an immunosuppressant or an inhibitor of assisted protein folding.

In some embodiments, the proteasome inhibitor is a proteasome-specific inhibitor; the first pharmaceutically active agent for treatment of a viral hepatitis infection is an interferon or a derivative thereof, an interleukin, a steroid, an immunomodulator, an immunosuppressant or an inhibitor of assisted protein folding; and the second pharmaceutically active agent for treatment of a viral hepatitis infection is an inhibitor of HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the first pharmaceutically active agent for treatment of a viral hepatitis infection is an interferon or a derivative thereof, a nucleoside analog or a nucleotide analog.

In some embodiments, the first pharmaceutically active agent for treatment of a viral hepatitis infection is an interferon or a derivative thereof; and the second pharmaceutically active agent for treatment of a viral hepatitis infection is a nucleoside analog or a nucleotide analog.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040 or a glycosylated, pegylated or hesylated form thereof.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon and Belerofon or a glycosylated, pegylated or hesylated form thereof; and the second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

In some embodiments, the first pharmaceutically active agent for treatment of a viral hepatitis infection is a pegylated form of interferon-alpha, beta, gamma or omega, or is ribavirin.

In some embodiments, the first pharmaceutically active agent for treatment of a viral hepatitis infection is a pegylated form of interferon-alpha, beta, gamma or omega; and the second pharmaceutically active agent for treatment of a viral hepatitis infection is ribavirin.

In some embodiments, the proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins, (−)-epigallocatechin-3-gallate, Disulfuram, acridine derivatives, tetra-acridine derivatives with betulinic acid, 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, TMV-95A endocyclic oxindole-phenyl clamp (BIA-1a) TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

In some embodiments, the proteasome inhibitor is PS-273, PS-341, PS-519 or S-2209.

In some embodiments, the proteasome inhibitor is PS-341, the first pharmaceutically active agent for treatment of a viral hepatitis infection is pegylated interferon alpha, and the second pharmaceutically active agent for treatment of a viral hepatitis infection nucleoside analog is ribavirin.

In some embodiments, the proteasome inhibitor is S-2209.

In some embodiments, the proteasome inhibitor is S-2209, wherein the first pharmaceutically active agent for treatment of a viral hepatitis infection is pegylated interferon alpha, and wherein the second pharmaceutically active agent for treatment of a viral hepatitis infection is ribavirin.

In some embodiments, the proteasome inhibitor and the first pharmaceutically active agent for treatment of a viral hepatitis infection are administered concurrently.

In some embodiments, the proteasome inhibitor, the first pharmaceutically active agent for treatment of a viral hepatitis infection and the second pharmaceutically active agent for treatment of a viral hepatitis infection are all administered concurrently.

In some embodiments, the proteasome inhibitor is administered first and the first pharmaceutically active agent for treatment of a viral hepatitis infection is administered subsequently after a delay.

In some embodiments, the proteasome inhibitor is administered first and the first pharmaceutically active agent for treatment of a viral hepatitis infection and the second pharmaceutically active agent for treatment of a viral hepatitis infection are administered subsequently after a delay.

In some embodiments, the first pharmaceutically active agent for treatment of a viral hepatitis infection is administered first and the proteasome inhibitor is administered subsequently after delay.

In some embodiments, the first pharmaceutically active agent for treatment of a viral hepatitis infection and the second pharmaceutically active agent for treatment of a viral hepatitis infection are administered first, and the proteasome inhibitor is administered subsequently after a delay.

In some embodiments, the delay is from about 2 weeks to about 8 weeks.

In some embodiments, the proteasome inhibitor is administered in one to two rounds of administrations, wherein each round of administration includes 3 to 10 administrations.

In some embodiments, the first pharmaceutically active agent for treatment of a viral hepatitis infection is administered for not more than about 50% of the duration otherwise recommended for the treatment of the hepatitis viral infection with the first pharmaceutically active agent and/or such that not more than about 66% of the dose recommended for the treatment of the viral hepatitis infection with the first pharmaceutically active agent is administered.

In some embodiments, the first and/or the second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the viral hepatitis infection with the respective pharmaceutically active agent is administered.

In some embodiments, the human or animal is ineligible for or unwilling to undergo treatment with a pharmaceutically active agent for treatment of a viral hepatitis infection.

In some embodiments, the hepatitis viral infection is an infection with Hepatitis C virus.

In one aspect, the invention provides a pharmaceutical composition comprising: an effective amount of PS-341 or S-2209; and an effective amount of a pegylated interferon-alpha.

In one embodiment, the pharmaceutical composition further comprises an effective amount of ribavirin.

In one aspect, the invention provides a kit for the treatment of a hepatitis viral infection, the kit comprising: a first pharmaceutical container containing a composition comprising an effective amount of a proteasome inhibitor; a second pharmaceutical container containing a composition comprising an effective amount of a first pharmaceutically active agent for treatment of a viral hepatitis infection; and instructions in paper or electronic form advising a user to first administer the composition comprising the proteasome inhibitor and administer the composition comprising the effective amount of first pharmaceutically active agent for treatment of a viral hepatitis infection subsequently after a delay.

In some embodiments, the second container further contains a composition comprising a second pharmaceutically active agent for treatment of a viral hepatitis infection which is different from the first pharmaceutically active agent for treatment of a viral hepatitis infection.

In some embodiments, the kit further comprises a third pharmaceutical container containing a composition comprising a second pharmaceutically active agent for treatment of a viral hepatitis infection which is different from the first pharmaceutically active agent for treatment of a viral hepatitis infection.

In one aspect, the invention provides a kit for the treatment of a hepatitis viral infection, the kit comprising: a first pharmaceutical container containing a composition comprising an effective amount of a proteasome inhibitor; a third pharmaceutical container containing a composition comprising an effective amount of a second pharmaceutically active agent for treatment of a viral hepatitis infection; and instructions in paper or electronic form advising a user to first administer the composition comprising the proteasome inhibitor and administer the composition comprising the effective amount of second pharmaceutically active agent for treatment of a viral hepatitis infection subsequently after a delay.

In some embodiments, the third container contains a composition comprising a first pharmaceutically active agent for treatment of a viral hepatitis infection which is different from the second pharmaceutically active agent for treatment of a viral hepatitis infection.

In some embodiments, the kit further comprises a second pharmaceutical container containing a composition comprising a first pharmaceutically active agent for treatment of a viral hepatitis infection which is different from the second pharmaceutically active agent for treatment of a viral hepatitis infection.

In some embodiments, the proteasome inhibitor is a proteasome-specific inhibitor; the first pharmaceutically active agent for treatment of a viral hepatitis infection is an agent which supports or assists a human or animal body's natural response in dealing with a viral infection; and the second pharmaceutically active agent for treatment of a viral hepatitis infection is an agent which interferes with the function of a viral target.

In some embodiments, the proteasome inhibitor is a proteasome-specific inhibitor; the first pharmaceutically active agent for treatment of a viral hepatitis infection is an interferon or a derivative thereof, an interleukin, a steroid, an immunomodulator, an immunosuppressant or an inhibitor of assisted protein folding; and the second pharmaceutically active agent for treatment of a viral hepatitis infection is an inhibitor of HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the protease inhibitor is a protease-specific inhibitor; the first pharmaceutically active agent for treatment of a viral hepatitis infection is an interferon or a derivative thereof; and the second pharmaceutically active agent for treatment of a viral hepatitis infection is a nucleoside analog or a nucleotide analog.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040, or a glycosylated, pegylated or hesylated form thereof; and the second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

In some embodiments, the first pharmaceutically active agent for treatment of a viral hepatitis infection is a pegylated form of interferon-alpha, beta, gamma or omega; and the second pharmaceutically active agent for treatment of a viral hepatitis infection is ribavirin.

In some embodiments, the proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins (−)-epigallocatechin-3-gallate, Disulfuram, acridine derivatives, tetra-acridine derivatives with betulinic acid, as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides, TMC-95A, TMC-95A endocyclic oxindole-phenyl clamp (BIA-1a) derivatives, TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

In some embodiments, the proteasome inhibitor is PS-273, PS-341, PS-519 or S-2209.

In some embodiments, the proteasome inhibitor is PS-341, the first pharmaceutically active agent for treatment of a viral hepatitis infection is pegylated interferon alpha, and the second pharmaceutically active agent for treatment of a viral hepatitis infection is ribavirin.

In some embodiments, the proteasome inhibitor is S-2209.

In some embodiments, the proteasome inhibitor is S-2209, the first pharmaceutically active agent for treatment of a viral hepatitis infection is pegylated interferon alpha, and the second pharmaceutically active agent for treatment of a viral hepatitis infection is ribavirin.

In some embodiments, the instructions advise the user to administer the compositions of the first and the second pharmaceutical containers concurrently.

In some embodiments, the instructions advise the user to first administer the composition of the first pharmaceutical container and administer the composition of the second pharmaceutical container subsequently after a delay.

In some embodiments, the instructions advise the user to first administer the composition of the second pharmaceutical container and administer the composition of the first pharmaceutical container subsequently after a delay.

In some embodiments, the instructions further advise the user of the length of the delay. In some embodiments, the delay is from about 2 weeks to about 8 weeks.

In some embodiments, the instructions advise the user to perform one to two rounds of administrations, each round consisting of 3 to 10 administrations of the composition of the first pharmaceutical container.

In some embodiments, the instructions advise the user to administer the first pharmaceutically active agent for treatment of a viral hepatitis infection for not more than about 50% of the duration otherwise recommended for the treatment of viral hepatitis infection with the first pharmaceutically active agent and/or to administer not more than about 66% of the dose recommended for the treatment of hepatitis viral infection with the first pharmaceutically active agent for treatment of a viral hepatitis infection.

In some embodiments, the instructions advise the user to apply the kit for the treatment of a human or animal individual ineligible for or unwilling to undergo treatment with a pharmaceutically active agent for treatment of a viral hepatitis infection.

In some embodiments, the hepatitis viral infection is an infection with Hepatitis C virus.

In another aspect, the invention provides a method for treating a hepatitis viral infection in a human or animal individual, the method comprising first administering to the human or animal in need thereof an effective amount of a proteasome inhibitor and administering an effective amount of a first pharmaceutically active agent for treatment of a viral hepatitis infection to the human or animal subsequently after a delay.

In some embodiments, the method further comprises administering to the human or animal an effective amount of a second pharmaceutically active agent for treatment of a viral hepatitis infection which is different from the first pharmaceutically active agent.

In another aspect, the invention provides a method for treating a hepatitis viral infection in a human or animal individual, the method comprising first administering to the human or animal in need thereof an effective amount of a proteasome inhibitor and administering an effective amount of a second pharmaceutically active agent for treatment of a viral hepatitis infection to the human or animal subsequently after a delay.

In some embodiments, the method further comprises administering to the human or animal an effective amount of a first pharmaceutically active agent for treatment of a viral hepatitis infection which is different from the second pharmaceutically active agent.

In some embodiments, the proteasome inhibitor is a proteasome-specific inhibitor; and the first pharmaceutically active agent for treatment of a viral hepatitis infection is an agent which supports or assists a human or animal body's natural response to a viral infection or an agent that interferes with the function of a viral target.

In some embodiments, the proteasome inhibitor is a proteasome-specific inhibitor; the first pharmaceutically active agent for treatment of a viral hepatitis infection is an agent which support or assists the human or animal body's natural response to a viral infection; and the second pharmaceutically active agent for treatment of a viral hepatitis infection is an agent that interferes with the function of a viral target.

In some embodiments, the proteasome inhibitor is a proteasome-specific inhibitor; and the first pharmaceutically active agent for treatment of a viral hepatitis infection is an interferon or a derivative thereof, an interleukin, a steroid, an immunomodulator, an immunosuppressant, an inhibitor of assisted protein folding, an inhibitor of HCV HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the proteasome inhibitor is a proteasome-specific inhibitor; the first pharmaceutically active agent for treatment of a viral hepatitis infection is an interferon or a derivative thereof, an interleukin, a steroid, an immunomodulator, an immunosuppressant or an inhibitor of assisted protein folding; and the second pharmaceutically active agent for treatment of a viral hepatitis infection is an inhibitor of HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the first pharmaceutically active agent for treatment of a viral hepatitis infection is an interferon or a derivative thereof, a nucleoside analog or a nucleotide analog.

In some embodiments, the first pharmaceutically active agent for treatment of a viral hepatitis infection is an interferon or a derivative thereof; and the second pharmaceutically active agent for treatment of a viral hepatitis infection is a nucleoside analog or a nucleotide analog.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040 or a glycosylated, pegylated or hesylated form thereof.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon and Belerofon or a glycosylated, pegylated or hesylated form thereof; and the second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

In some embodiments, the first pharmaceutically active agent for treatment of a viral hepatitis infection is a pegylated form of interferon-alpha, beta, gamma or omega, or is ribavirin.

In some embodiments, the first pharmaceutically active agent for treatment of a viral hepatitis infection is a pegylated form of interferon-alpha, beta, gamma or omega; and the second pharmaceutically active agent for treatment of a viral hepatitis infection is ribavirin.

In some embodiments, the proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins, (−)-epigallocatechin-3-gallate, Disulfuram, acridine derivatives, tetra-acridine derivatives with betulinic acid, 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, TMV-95A endocyclic oxindole-phenyl clamp (BIA-1a) TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

In some embodiments, the proteasome inhibitor is PS-273, PS-341, PS-519 or S-2209.

In some embodiments, the proteasome inhibitor is PS-341, the first pharmaceutically active agent for treatment of a viral hepatitis infection is pegylated interferon alpha, and the second pharmaceutically active agent for treatment of a viral hepatitis infection nucleoside analog is ribavirin.

In some embodiments, the proteasome inhibitor is S-2209.

In some embodiments, the proteasome inhibitor is S-2209, wherein the first pharmaceutically active agent for treatment of a viral hepatitis infection is pegylated interferon alpha, and wherein the second pharmaceutically active agent for treatment of a viral hepatitis infection is ribavirin.

In some embodiments, the proteasome inhibitor and the first pharmaceutically active agent for treatment of a viral hepatitis infection are administered concurrently.

In some embodiments, the proteasome inhibitor, the first pharmaceutically active agent for treatment of a viral hepatitis infection and the second pharmaceutically active agent for treatment of a viral hepatitis infection are all administered concurrently.

In some embodiments, the proteasome inhibitor is administered first and the first pharmaceutically active agent for treatment of a viral hepatitis infection is administered subsequently after a delay.

In some embodiments, the proteasome inhibitor is administered first and the first pharmaceutically active agent for treatment of a viral hepatitis infection and the second pharmaceutically active agent for treatment of a viral hepatitis infection are administered subsequently after a delay.

In some embodiments, the first pharmaceutically active agent for treatment of a viral hepatitis infection is administered first and the proteasome inhibitor is administered subsequently after delay.

In some embodiments, the first pharmaceutically active agent for treatment of a viral hepatitis infection and the second pharmaceutically active agent for treatment of a viral hepatitis infection are administered first, and the proteasome inhibitor is administered subsequently after a delay.

In some embodiments, the delay is from about 2 weeks to about 8 weeks.

In some embodiments, the proteasome inhibitor is administered in one to two rounds of administrations, wherein each round of administration includes 3 to 10 administrations.

In some embodiments, the first pharmaceutically active agent for treatment of a viral hepatitis infection is administered for not more than about 50% of the duration otherwise recommended for the treatment of the hepatitis viral infection with the first pharmaceutically active agent and/or such that not more than about 66% of the dose recommended for the treatment of the viral hepatitis infection with the first pharmaceutically active agent is administered.

In some embodiments, the first and/or the second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the viral hepatitis infection with the respective pharmaceutically active agent is administered.

In some embodiments, the human or animal is ineligible for or unwilling to undergo treatment with a pharmaceutically active agent for treatment of a viral hepatitis infection.

In some embodiments, the hepatitis viral infection is an infection with Hepatitis C virus.

In one aspect, the invention provides a pharmaceutical composition comprising: an effective amount of PS-341 or S-2209; and an effective amount of a pegylated interferon-alpha.

In one embodiment, the pharmaceutical composition further comprises ribavirin.

In one aspect, the invention provides a pharmaceutical composition comprising an effective amount of a proteasome inhibitor and an effective amount of a first and/or a second pharmaceutically active agent for treatment of a viral hepatitis infection.

In some embodiments, the proteasome inhibitor is a proteasome-specific inhibitor; and the first pharmaceutically active agent for treatment of a viral hepatitis infection is an agent which supports or assists a human or animal body's natural response to a viral infection or an agent that interferes with the function of a viral target.

In some embodiments, the proteasome inhibitor is a proteasome-specific inhibitor; the first pharmaceutically active agent for treatment of a viral hepatitis infection is an agent which support or assists the human or animal body's natural response to a viral infection; and the second pharmaceutically active agent for treatment of a viral hepatitis infection is an agent that interferes with the function of a viral target.

In some embodiments, the proteasome inhibitor is a proteasome-specific inhibitor; and the first pharmaceutically active agent for treatment of a viral hepatitis infection is an interferon or a derivative thereof, an interleukin, a steroid, an immunomodulator, an immunosuppressant, an inhibitor of assisted protein folding, an inhibitor of HCV HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the proteasome inhibitor is a proteasome-specific inhibitor; the first pharmaceutically active agent for treatment of a viral hepatitis infection is an interferon or a derivative thereof, an interleukin, a steroid, an immunomodulator, an immunosuppressant or an inhibitor of assisted protein folding; and the second pharmaceutically active agent for treatment of a viral hepatitis infection is an inhibitor of HCV HCV NS3/4A protease, HCV NS4B protein, HCV NS5A protein, HCV NS5B polymerase or of an HCV envelope protein.

In some embodiments, the first pharmaceutically active agent for treatment of a viral hepatitis infection is an interferon or a derivative thereof, a nucleoside analog or a nucleotide analog.

In some embodiments, the first pharmaceutically active agent for treatment of a viral hepatitis infection is an interferon or a derivative thereof; and the second pharmaceutically active agent for treatment of a viral hepatitis infection is a nucleoside analog or a nucleotide analog.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040 or a glycosylated, pegylated or hesylated form thereof.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon and Belerofon or a glycosylated, pegylated or hesylated form thereof; and the second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

In some embodiments, the first pharmaceutically active agent for treatment of a viral hepatitis infection is a pegylated form of interferon-alpha, beta, gamma or omega, or is ribavirin.

In some embodiments, the first pharmaceutically active agent for treatment of a viral hepatitis infection is a pegylated form of interferon-alpha, beta, gamma or omega; and the second pharmaceutically active agent for treatment of a viral hepatitis infection is ribavirin. In some embodiments, the proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins, (−)-epigallocatechin-3-gallate, Disulfuram, acridine derivatives, tetra-acridine derivatives with betulinic acid, 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, TMV-95A endocyclic oxindole-phenyl clamp (BIA-1a) TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

In some embodiments, the proteasome inhibitor is PS-273, PS-341, PS-519 or S-2209.

In some embodiments, the proteasome inhibitor is PS-341, the first pharmaceutically active agent for treatment of a viral hepatitis infection is pegylated interferon alpha, and the second pharmaceutically active agent for treatment of a viral hepatitis infection nucleoside analog is ribavirin.

In some embodiments, the proteasome inhibitor is S-2209.

In some embodiments, the proteasome inhibitor is S-2209, wherein the first pharmaceutically active agent for treatment of a viral hepatitis infection is pegylated interferon alpha, and wherein the second pharmaceutically active agent for treatment of a viral hepatitis infection is ribavirin.

In one embodiment, the pharmaceutical composition further comprises ribavirin.

In one aspect, the invention provides a kit for the treatment of a hepatitis viral infection, the kit comprising: a first pharmaceutical container containing a composition comprising an effective amount of a proteasome inhibitor; a second pharmaceutical container containing a composition comprising an effective amount of a first pharmaceutically active agent for treatment of a viral hepatitis infection; and instructions in paper or electronic form advising the user to administer the first pharmaceutically active agent for treatment of a viral hepatitis infection for a reduced duration compared to the duration otherwise recommended for the treatment of viral hepatitis infection with the first pharmaceutically active agent and/or to administer a reduced dose compared to the dose recommended for the treatment of hepatitis viral infection with the first pharmaceutically active agent for treatment of a viral hepatitis infection.

In some embodiments, the second container further contains a composition comprising an effective amount of a second pharmaceutically active agent for treatment of a viral hepatitis infection which is different from the first pharmaceutically active agent for treatment of a viral hepatitis infection.

In some embodiments, the kit further comprises a third pharmaceutical container containing a composition comprising an effective amount of a second pharmaceutically active agent for treatment of a viral hepatitis infection which is different from the first pharmaceutically active agent for treatment of a viral hepatitis infection.

In one aspect, the invention provides a kit for the treatment of a hepatitis viral infection, the kit comprising: a first pharmaceutical container containing a composition comprising an effective amount of a proteasome inhibitor; a third pharmaceutical container containing a composition comprising an effective amount of a second pharmaceutically active agent for treatment of a viral hepatitis infection; and instructions in paper or electronic form advising the user to administer the second pharmaceutically active agent for treatment of a viral hepatitis infection for a reduced duration compared to the duration otherwise recommended for the treatment of viral hepatitis infection with the second pharmaceutically active agent and/or to administer a reduced dose compared to the dose recommended for the treatment of hepatitis viral infection with the second pharmaceutically active agent for treatment of a viral hepatitis infection.

In some embodiments, the third container contains a composition comprising an effective amount of a first pharmaceutically active agent for treatment of a viral hepatitis infection which is different from the second pharmaceutically active agent for treatment of a viral hepatitis infection.

In some embodiments, the kit further comprises a second pharmaceutical container containing a composition comprising an effective amount of a first pharmaceutically active agent for treatment of a viral hepatitis infection which is different from the second pharmaceutically active agent for treatment of a viral hepatitis infection.

In some embodiments, the proteasome inhibitor is a proteasome-specific inhibitor; the first pharmaceutically active agent for treatment of a viral hepatitis infection is an agent which supports or assists a human or animal body's natural response in dealing with a viral infection; and the second pharmaceutically active agent for treatment of a viral hepatitis infection is an agent which interferes with the function of a viral target.

In some embodiments, the proteasome inhibitor is a proteasome-specific inhibitor; the first pharmaceutically active agent for treatment of a viral hepatitis infection is an interferon or a derivative thereof, an interleukin, a steroid, an immunomodulator, an immunosuppressant or an inhibitor of assisted protein folding; and the second pharmaceutically active agent for treatment of a viral hepatitis infection is an inhibitor of HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the protease inhibitor is a protease-specific inhibitor; the first pharmaceutically active agent for treatment of a viral hepatitis infection is an interferon or a derivative thereof; and the second pharmaceutically active agent for treatment of a viral hepatitis infection is a nucleoside analog or a nucleotide analog.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040, or a glycosylated, pegylated or hesylated form thereof; and the second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

In some embodiments, the first pharmaceutically active agent for treatment of a viral hepatitis infection is a pegylated form of interferon-alpha, beta, gamma or omega; and the second pharmaceutically active agent for treatment of a viral hepatitis infection is ribavirin.

In some embodiments, the proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins (−)-epigallocatechin-3-gallate, Disulfuram, acridine derivatives, tetra-acridine derivatives with betulinic acid, as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides, TMC-95A, TMC-95A endocyclic oxindole-phenyl clamp (BIA-1a) derivatives, TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

In some embodiments, the proteasome inhibitor is PS-273, PS-341, PS-519 or S-2209.

In some embodiments, the proteasome inhibitor is PS-341, the first pharmaceutically active agent for treatment of a viral hepatitis infection is pegylated interferon alpha, and the second pharmaceutically active agent for treatment of a viral hepatitis infection is ribavirin.

In some embodiments, the proteasome inhibitor is S-2209.

In some embodiments, the proteasome inhibitor is S-2209, the first pharmaceutically active agent for treatment of a viral hepatitis infection is pegylated interferon alpha, and the second pharmaceutically active agent for treatment of a viral hepatitis infection is ribavirin.

In some embodiments, the instructions advise the user to administer the compositions of the first and the second pharmaceutical containers concurrently.

In some embodiments, the instructions advise the user to first administer the composition of the first pharmaceutical container and administer the composition of the second pharmaceutical container subsequently after a delay.

In some embodiments, the instructions advise the user to first administer the composition of the second pharmaceutical container and administer the composition of the first pharmaceutical container subsequently after a delay.

In some embodiments, the instructions further advise the user of the length of the delay. In some embodiments, the delay is from about 2 weeks to about 8 weeks.

In some embodiments, the instructions advise the user to perform one to two rounds of administrations, each round consisting of 3 to 10 administrations of the composition of the first pharmaceutical container.

In some embodiments, the instructions advise the user to apply the kit for the treatment of a human or animal individual ineligible for or unwilling to undergo treatment with a pharmaceutically active agent for treatment of a viral hepatitis infection.

In some embodiments, the hepatitis viral infection is an infection with Hepatitis C virus.

In another aspect, the invention provides a method for treating a hepatitis viral infection in a human or animal individual who does not respond or is refractory to treatment with a pharmaceutically active agent for treatment of viral hepatitis infection, the method comprising administering to the human or animal in need thereof a proteasome inhibitor and a first pharmaceutically active agent for treatment of a viral hepatitis infection.

In some embodiments, the method further comprises administering to the human or animal a second pharmaceutically active agent for treatment of a viral hepatitis infection which is different from the first pharmaceutically active agent.

In another aspect, the invention provides a method for treating a hepatitis viral infection in a human or animal individual who does not respond or is refractory to treatment with a pharmaceutically active agent for treatment of viral hepatitis infection, the method comprising administering to the human or animal in need thereof a proteasome inhibitor and a second pharmaceutically active agent for treatment of a viral hepatitis infection.

In some embodiments, the method further comprises administering to the human or animal a first pharmaceutically active agent for treatment of a viral hepatitis infection which is different from the second pharmaceutically active agent.

In some embodiments, the proteasome inhibitor is a proteasome-specific inhibitor; and the first pharmaceutically active agent for treatment of a viral hepatitis infection is an agent which supports or assists a human or animal body's natural response to a viral infection or an agent that interferes with the function of a viral target.

In some embodiments, the proteasome inhibitor is a proteasome-specific inhibitor; the first pharmaceutically active agent for treatment of a viral hepatitis infection is an agent which support or assists the human or animal body's natural response to a viral infection; and the second pharmaceutically active agent for treatment of a viral hepatitis infection is an agent that interferes with the function of a viral target.

In some embodiments, the proteasome inhibitor is a proteasome-specific inhibitor; and the first pharmaceutically active agent for treatment of a viral hepatitis infection is an interferon or a derivative thereof, an interleukin, a steroid, an immunomodulator, an immunosuppressant, an inhibitor of assisted protein folding, an inhibitor of HCV HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the proteasome inhibitor is a proteasome-specific inhibitor; the first pharmaceutically active agent for treatment of a viral hepatitis infection is an interferon or a derivative thereof, an interleukin, a steroid, an immunomodulator, an immunosuppressant or an inhibitor of assisted protein folding; and the second pharmaceutically active agent for treatment of a viral hepatitis infection is an inhibitor of HCV NS3/4A protease, an inhibitor of HCV NS4B protein, an inhibitor of HCV NS5A protein, an inhibitor of HCV NS5B polymerase or an inhibitor of an HCV envelope protein.

In some embodiments, the first pharmaceutically active agent for treatment of a viral hepatitis infection is an interferon or a derivative thereof, a nucleoside analog or a nucleotide analog.

In some embodiments, the first pharmaceutically active agent for treatment of a viral hepatitis infection is an interferon or a derivative thereof; and the second pharmaceutically active agent for treatment of a viral hepatitis infection is a nucleoside analog or a nucleotide analog.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040 or a glycosylated, pegylated or hesylated form thereof.

In some embodiments, the first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon and Belerofon or a glycosylated, pegylated or hesylated form thereof; and the second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

In some embodiments, the first pharmaceutically active agent for treatment of a viral hepatitis infection is a pegylated form of interferon-alpha, beta, gamma or omega, or is ribavirin.

In some embodiments, the first pharmaceutically active agent for treatment of a viral hepatitis infection is a pegylated form of interferon-alpha, beta, gamma or omega; and the second pharmaceutically active agent for treatment of a viral hepatitis infection is ribavirin.

In some embodiments, the proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins, (−)-epigallocatechin-3-gallate, Disulfuram, acridine derivatives, tetra-acridine derivatives with betulinic acid, 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, TMV-95A endocyclic oxindole-phenyl clamp (BIA-1a) TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

In some embodiments, the proteasome inhibitor is PS-273, PS-341, PS-519 or S-2209.

In some embodiments, the proteasome inhibitor is PS-341, the first pharmaceutically active agent for treatment of a viral hepatitis infection is pegylated interferon alpha, and the second pharmaceutically active agent for treatment of a viral hepatitis infection nucleoside analog is ribavirin.

In some embodiments, the proteasome inhibitor is S-2209.

In some embodiments, the proteasome inhibitor is S-2209, wherein the first pharmaceutically active agent for treatment of a viral hepatitis infection is pegylated interferon alpha, and wherein the second pharmaceutically active agent for treatment of a viral hepatitis infection is ribavirin.

In some embodiments, the proteasome inhibitor and the first pharmaceutically active agent for treatment of a viral hepatitis infection are administered concurrently.

In some embodiments, the proteasome inhibitor, the first pharmaceutically active agent for treatment of a viral hepatitis infection and the second pharmaceutically active agent for treatment of a viral hepatitis infection are all administered concurrently.

In some embodiments, the proteasome inhibitor is administered first and the first pharmaceutically active agent for treatment of a viral hepatitis infection is administered subsequently after a delay.

In some embodiments, the proteasome inhibitor is administered first and the first pharmaceutically active agent for treatment of a viral hepatitis infection and the second pharmaceutically active agent for treatment of a viral hepatitis infection are administered subsequently after a delay.

In some embodiments, the first pharmaceutically active agent for treatment of a viral hepatitis infection is administered first and the proteasome inhibitor is administered subsequently after delay.

In some embodiments, the first pharmaceutically active agent for treatment of a viral hepatitis infection and the second pharmaceutically active agent for treatment of a viral hepatitis infection are administered first, and the proteasome inhibitor is administered subsequently after a delay.

In some embodiments, the delay is from about 2 weeks to about 8 weeks.

In some embodiments, the proteasome inhibitor is administered in one to two rounds of administrations, wherein each round of administration includes 3 to 10 administrations.

In some embodiments, the first pharmaceutically active agent for treatment of a viral hepatitis infection is administered for not more than about 95%, 90% 85%, 80%, 75% 70%, 66%, 50%, 40%, 33%, 25% 15% or 10% of the duration otherwise recommended for the treatment of the hepatitis viral infection with the first pharmaceutically active agent and/or such that not more than about 95%, 90% 85%, 80%, 75% 70%, 66%, 50%, 40%, 33%, 25% 15% or 10% of the dose recommended for the treatment of the viral hepatitis infection with the first pharmaceutically active agent is administered.

In some embodiments, the first and/or the second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the viral hepatitis infection with the respective pharmaceutically active agent is administered.

In some embodiments, the human or animal is ineligible for or unwilling to undergo treatment with a pharmaceutically active agent for treatment of a viral hepatitis infection.

In some embodiments, the hepatitis viral infection is an infection with Hepatitis C virus.

In another aspect, the invention relates to a method of treating a hepatitis C infections comprising administering to a patient in need thereof an effective amount of a proteasome inhibitor, an interferon or a derivative thereof, and a nucleoside analog.

In some embodiments, the hepatitis C infection is a hepatitis C infection resistant to treatment with interferons and nucleoside analogs.

In some embodiments, the interferon alpha, interferon beta or interferon gamma. In some embodiments, the interferon is pegylated.

In some embodiments, the pegylated interferon is pegylated interferon alpha, pegylated interferon beta or pegylated interferon gamma.

In some embodiments, the pegylated interferon is pegylated interferon alpha-2a or pegylated interferon alpha-2b.

In some embodiments, the proteasome inhibitor is one of the following classes of compounds:

(a) Naturally occurring proteasome inhibitors: Peptide derivatives, comprising C-terminal epoxyketone groups; or Beta-lactone derivatives; or Aclacinomycin A (also known as Aclarubicin); or Lactacystin and its chemically modified variants, such as the cell membrane penetrating variant “Clasto lactacystin beta-lactone”; (b) Synthetically prepared proteasome inhibitors: Modified peptide aldehydes, such as N-carbobenzoxy-L-leucinyl-L-leucinyl-L-leucinal (also known as MG132 or zLLL), its boric acid derivative MG232; N-Carbobenzoxy-Leu-Leu-Nva-H (also known as MG115; N-AcetylL-Leuzinyl-L-Leuzinyl-L-Norleuzinal (also known as LLnL), N-Carbobenzoxy-Ile-Glu(OBut)-Ala-Leu-H (also known as PSI); (c) Peptides with C-terminal α,β-Epoxyketone groups; vinyl sulfones, e.g., Carbobenzoxy-L-Leucinyl-L-Leucinyl-L-Leucin-vinyl sulfone or 4-Hydroxy-5-iodo-3-nitrophenylactetyl-L-Leucinyl-L-Leucinyl-L-Leucin-vinyl sulfone (NLVS); (d) Glyoxal- or boric acid groups, e.g., Pyrazyl-CONH(CHPhe)CONH(CHisobutyl)B(OH)₂) and boric acid derivatized dipeptides; (e) Pinacol esters—e.g., Benzyloxycarbonyl(Cbz)-Leu-Leu-boroLeu-pinacol ester.

In another embodiment, proteasome inhibitor is: one of the epoxyketones such as epoxomicin (also known as epoxomycin, molecular formula: C₂₈H₈₆N₄O₇) and/or eponemycin (also known as eponemicin, molecular formula: C₂₀H₃₆N₂O₅).

In some embodiments, the proteasome inhibitor is: (a) PS-519 as beta-Lacton- as well as a derivative of Lactacystin the compound 1R-[1S,4R,5S]]-1-(1-Hydroxy-2-methylpropyl)-4-propyl-6-oxa-2-azabicyclo[3.2.0]heptane-3,7-dione—molecular formula C₁₂H₁₉NO₄—and/or (b) PS-314 as boric acid peptide derivative the compound N-Pyrazinecarbonyl-L-Phenylalanin-L-Leuzin-boric acid—molecular formula C₁₉H₂₅BN₄O₄—and/or (c) PS-273 (Morpholin-CONH—(CH-Naphthyl)-CONH—(CH-isobutyl)-B(OH)₂) and its enantiomer PS-293 and/or (d) PS-296 (8-Quinolyl-sulfonyl-CONH—(CH-Napthyl)-CONH(—CH-isobutyl)-B(OH)₂) and/or (e) PS-303 (NH₂(CH-Naphthyl)-CONH—(CH-isobutyl)-B(OH)₂) and/or (f) PS-321 as (Morpholin-CONH—(CH-Napthyl)-CONH—(CH-Phenylalanin)-B(OH)₂); -and/or (g) PS-334 (CH₃—NH—(CH-Naphthyl-CONH—(CH-Isobutyl)-B(OH)₂) and/or (h) PS-325 (2-Quinol-CONH—(CH-homo-Phenylalanin)-CONH—(CH-isobutyl)-B(OH)₂) and/or (i) PS-352 (Phenyalanin-CH₂—CH₂—CONH—(CH-Phenylalanin)-CONH—(CH-isobutyl)-B(OH)₂) and/or (j) PS-383 (Pyridyl-CONH—(CHpF-Phenylalanin)-CONH—(CH-isobutyl)-B(OH)₂).

In some embodiments, the concentration of the proteasome inhibitor in the peripheral blood or cytoplasm is in the range of 1 nM to 100 μM.

In some embodiments, the concentration of the interferon is 180 μg/week for PEGASYS (Roche) and 1.5 μg/kg for PegIntron (Essex).

In some embodiments, the concentration of the nucleoside analog (Rebetol, Essex) is 800-1200 mg/day (weight corrected).

The above embodiments can be combined with each other and/or with any of the above aspects.

### DETAILED DESCRIPTION OF THE INVENTION

In a first embodiment, the invention relates to a kit of pharmaceutical compositions for the treatment of a hepatitis viral infection in a human or animal individual who does not respond or is refractory to treatment with at least one pharmaceutically active agent for treatment of a viral hepatitis infection, comprising: at least one first pharmaceutical composition comprising at least one proteasome inhibitor; and at least one second pharmaceutical composition comprising at least one first pharmaceutically active agent for treatment of a viral hepatitis infection, and optionally at least one second pharmaceutically active agent for treatment of a viral hepatitis infection, present in said at least one second or in at least one third pharmaceutical container.

In one embodiment, the invention relates to a kit, wherein said kit comprises said at least one second pharmaceutically active agent for treatment of a viral hepatitis infection present in said at least one second or in at least one third pharmaceutical container.

In one embodiment, the invention relates to a kit, wherein: said at least one proteasome inhibitor of said first pharmaceutical composition is selected from the group comprising proteasome-specific inhibitors; said at least one first pharmaceutically active agent for treatment of a viral hepatitis infection of said at least one second pharmaceutical container is selected from one of (A) the group comprising agents which support or assist the body's natural response in dealing with viral infections, or (B) the group comprising agents which interfere with the function of a viral target; and said at least one second pharmaceutically active agent for treatment of a viral hepatitis infection, where present, is selected from (B) if the first pharmaceutically active agent for treatment of a viral hepatitis infection is selected from (A), or selected from (A) if the first pharmaceutically active agent for treatment of a viral hepatitis infection is selected from (B).

In one embodiment, the invention relates to a kit, wherein: said at least one proteasome inhibitor of said first pharmaceutical composition is selected from the group comprising proteasome-specific inhibitors; said group (A) comprising agents which support or assist the body's natural response in dealing with viral infections comprises interferons including their derivatives, interleukins, steroids, immunomodulators, immunosuppressants, and inhibitors of assisted protein folding; and said group (B) comprising agents which interfere with the function of a viral target comprises inhibitors of HCV NS3/4A protease, HCV NS4B protein, HCV NS5A protein, HCV NS5B polymerase and HCV envelope proteins.

In one embodiment, the invention relates to a kit, wherein: said group (A) comprising agents which support or assist the body's natural response in dealing with viral infections comprises interferons including their derivatives; and said group (B) comprising agents which interfere with the function of a viral target comprises nucleoside or nucleotide analogs.

In one embodiment, the invention relates to a kit, wherein: said group (A) comprising agents which support or assist the body's natural response in dealing with viral infections consists of: interferon-alpha, beta, gamma or omega, albinterferon, Locteron, Omega IFN, Medusa IFN, DA-3021, EMZ702, Infradure, IL28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040 and the glycosylated, pegylated or hesylated forms thereof; and said group (B) comprising agents which interfere with the function of a viral target consists of: lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222, PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132).

In one embodiment, the invention relates to a kit, wherein: said group (A) comprising agents which support or assist the body's natural response in dealing with viral infections consists of pegylated forms of interferon-alpha, beta, gamma or omega; and said group (B) comprising agents which interfere with the function of a viral target consists of: is ribavirin.

In one embodiment, the invention relates to a kit, wherein said at least one proteasome inhibitor of said first pharmaceutical container is selected from the group comprising: peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (also known as aclarubicine), celastrol, withaferin A, Gliotoxin, epipolythiodioxo-piperazines, green tea polyphenolic catechins such as (−)-epigallocatechin-3-gallate, Disulfuram, acridine derivatives including tetra-acridine derivatives with betulinic acid such as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, its endocyclic oxindole-phenyl clamp (BIA-1a) and endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal, its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer “PS-293”, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209 and its structural analogs Compound 1-6 and Compound 8.

In one embodiment, the invention relates to a kit, wherein said at least one proteasome inhibitor present in said first pharmaceutical container is PS-273, PS-341, PS-519, or S-2209.

In one embodiment, the invention relates to a kit, wherein said at least one proteasome inhibitor of said first pharmaceutical container is PS-341, wherein said interferon is pegylated interferon alpha, and wherein said nucleoside analog is ribavirin.

In one embodiment, the invention relates to a kit, wherein said at least one proteasome inhibitor of said first pharmaceutical container is S-2209.

In one embodiment, the invention relates to a kit, wherein said at least one proteasome inhibitor of said first pharmaceutical container is S-2209, wherein said interferon is pegylated interferon alpha, and wherein said nucleoside analog is ribavirin.

In one embodiment, the invention relates to a kit, further comprising instructions in paper or electronic form advising the user to apply the kit for the treatment of a human or animal individual who does not respond or is refractory to treatment with at least one pharmaceutically active agent for treatment of a viral hepatitis infection.

In one embodiment, the invention relates to a kit, further comprising instructions in paper or electronic form advising the user to administer the compositions of the first pharmaceutical container and the composition of the second and, where present, third pharmaceutical container concurrently.

In one embodiment, the invention relates to a kit, further comprising instructions in paper or electronic form advising the user to first administer the compositions of the first pharmaceutical container and administer the compositions of the second and, where present, third pharmaceutical container subsequently after a delay.

In one embodiment, the invention relates to a kit, further comprising instructions in paper or electronic form advising the user to first administer the compositions of the second and, where present, third pharmaceutical container(s), and administer the compositions of the first pharmaceutical container subsequently after a delay.

In one embodiment, the invention relates to a kit, wherein the instructions further advise the user on a specified time period for said delay.

In one embodiment, the invention relates to a kit, wherein said specified time period is more than about 2 weeks, but less than about 8 weeks.

In one embodiment, the invention relates to a kit, further comprising instructions in paper or electronic form advising the user to perform one to two rounds of administrations, each round consisting of 3 to 10 administrations, of the compositions of the first pharmaceutical composition comprising a proteasome inhibitor.

In one embodiment, the invention relates to a kit, further comprising instructions in paper or electronic form advising the user to administer at least one of the at least one first and/or, where present, at least one second pharmaceutically active agent for treatment of a viral hepatitis infection for not more than about 50% of the duration otherwise recommended for the treatment of the hepatitis viral infection with the respective pharmaceutically active agent for treatment of a viral hepatitis infection and/or to administer not more than about 66% of the dose recommended for the treatment of the hepatitis viral infection with the respective pharmaceutically active agent for treatment of a viral hepatitis infection.

In one embodiment, the invention relates to a kit, further comprising instructions in paper or electronic form advising the user to apply the kit for the treatment of a human or animal individual ineligible for or unwilling to undergo treatment with at least one pharmaceutically active agent for treatment of a viral hepatitis infection.

In one embodiment, the invention relates to a kit, wherein the hepatitis viral infection is an infection with Hepatitis C virus.

In a second embodiment, the invention relates to a use of at least one proteasome inhibitor and at least one first pharmaceutically active agent for treatment of a viral hepatitis infection, and optionally at least one second pharmaceutically active agent for treatment of a viral hepatitis infection, in the manufacture of a pharmaceutical composition for the treatment of a hepatitis viral infection in a human or animal individual who does not respond or is refractory to treatment with at least one pharmaceutically active agent for treatment of a viral hepatitis infection.

In one embodiment, the invention relates to a use, wherein said at least one second pharmaceutically active agent for treatment of a viral hepatitis infection is used in said manufacture of a pharmaceutical composition.

In one embodiment, the invention relates to a use, wherein said at least one proteasome inhibitor is selected from the group comprising proteasome-specific inhibitors; said at least one first pharmaceutically active agent for treatment of a viral hepatitis infection is selected from one of (A) the group comprising agents which support or assist the body's natural response in dealing with viral infections, or (B) the group comprising agents which interfere with the function of a viral target; and said at least one second pharmaceutically active agent for treatment of a viral hepatitis infection, where present, is selected from (B) if the first pharmaceutically active agent for treatment of a viral hepatitis infection is selected from (A), or selected from (A) if the first pharmaceutically active agent for treatment of a viral hepatitis infection is selected from (B).

In one embodiment, the invention relates to a use, wherein said at least one proteasome inhibitor of said first pharmaceutical composition is selected from the group comprising proteasome-specific inhibitors; said group (A) comprising agents which support or assist the body's natural response in dealing with viral infections comprises interferons including their derivatives, interleukins, steroids, immunomodulators, immunosuppressants, and inhibitors of assisted protein folding; and said group (B) comprising agents which interfere with the function of a viral target comprises inhibitors of HCV NS3/4A protease, HCV NS4B protein, HCV NS5A protein, HCV NS5B polymerase and HCV envelope proteins.

In one embodiment, the invention relates to a use, wherein said group (A) comprising agents which support or assist the body's natural response in dealing with viral infections comprises interferons including their derivatives; and said group (B) comprising agents which interfere with the function of a viral target comprises nucleoside or nucleotide analogs.

In one embodiment, the invention relates to a use, wherein said group (A) comprising agents which support or assist the body's natural response in dealing with viral infections consists of: interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD-101, IMO-2125, GI-5005 (Tarmogen), IC41, PF-04878691, TG 4040 and the glycosylated, pegylated or hesylated forms thereof; and said group (B) comprising agents which interfere with the function of a viral target consists of: lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222, PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132).

In one embodiment, the invention relates to a use, wherein said group (A) comprising agents which support or assist the body's natural response in dealing with viral infections consists of: pegylated forms of interferon-alpha, beta, gamma or omega; and said at least one second pharmaceutically active agent for treatment of a viral hepatitis infection, where present, is ribavirin.

In one embodiment, the invention relates to a use, wherein said at least one proteasome inhibitor is selected from the group consisting of: peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (also known as aclarubicine), celastrol, withaferin A, Gliotoxin, epipolythiodioxo-piperazines, green tea polyphenolic catechins such as (−)-epigallocatechin-3-gallate, Disulfuram, acridine derivatives including tetra-acridine derivatives with betulinic acid such as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, its endocyclic oxindole-phenyl clamp (BIA-1a) and endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal, its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer “PS-293”, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209 and its structural analogs Compound 1-6 and Compound 8.

In one embodiment, the invention relates to a use, wherein said inhibitor is PS-273, PS-341, PS-519, or S-2209.

In one embodiment, the invention relates to a use, wherein said at least one proteasome inhibitor is PS-341, wherein said interferon is pegylated interferon alpha, and wherein said nucleoside analog is ribavirin.

In one embodiment, the invention relates to a use, wherein said at least one proteasome inhibitor is S-2209.

In one embodiment, the invention relates to a use, wherein said at least one proteasome inhibitor is S-2209, wherein said interferon is pegylated interferon alpha, and wherein said nucleoside analog is ribavirin.

In one embodiment, the invention relates to a use, wherein the pharmaceutical composition is provided in a manner such that the proteasome inhibitor, the first pharmaceutically active agent for treatment of a viral hepatitis infection and, where present, the second pharmaceutically active agent for treatment of a viral hepatitis infection are administered concurrently.

In one embodiment, the invention relates to a use, wherein the pharmaceutical composition is provided in a manner such that the proteasome inhibitor is administered first, and the first pharmaceutically active agent for treatment of a viral hepatitis infection and, where present, the second pharmaceutically active agent for treatment of a viral hepatitis infection are administered subsequently with delay.

In one embodiment, the invention relates to a use, wherein the pharmaceutical composition is provided in a manner such that the first pharmaceutically active agent for treatment of a viral hepatitis infection and, where present, the second pharmaceutically active agent for treatment of a viral hepatitis infection are administered first, and the proteasome inhibitor is administered subsequently after a delay.

In one embodiment, the invention relates to a use, wherein said delay is a specified time period.

In one embodiment, the invention relates to a use, wherein said specified time period is more than about 2 weeks, but less than about 8 weeks, or is 2-8 weeks. As far as the eighth through fourteenth embodiments as described hereinafter mention suitable specified time periods for subsequent administration, these specified time periods also apply to the first to seventh embodiment.

In one embodiment, the invention relates to a use, wherein the pharmaceutical composition is provided in a manner such that one to two rounds of administrations, each round consisting of 3 to 10 administrations, of the at least one proteasome inhibitor are performed.

In one embodiment, the invention relates to a use, wherein at least one of said at least one first and/or, where present, at least one second pharmaceutically active agent for treatment of a viral hepatitis infection is provided such that the at least one first and/or, where present, at least one second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the hepatitis viral infection with the respective pharmaceutically active agent for treatment of a viral hepatitis infection is administered. In one embodiment, the invention relates to a use, wherein the pharmaceutical composition is provided for the treatment of a human or animal individual ineligible for or unwilling to undergo treatment with at least one pharmaceutically active agent for treatment of a viral hepatitis infection.

In one embodiment, the invention relates to a use, wherein the hepatitis viral infection is an infection with Hepatitis C virus.

In a third embodiment, the invention relates to pharmaceutical compositions essentially as provided by the second embodiment's uses described herein.

If in the context of the present invention it is stated that the invention relates to a use of an active ingredient such as e.g., at least one proteasome inhibitor and e.g., at least one first pharmaceutically active agent for treatment of a viral hepatitis infection in the manufacture of a pharmaceutical composition e.g., for treating a specific condition, e.g., for treating a specific condition in a selected group of patients, e.g., for using a specific dosage regimen in the treatment of a specific condition, etc., then such statements are meant to also disclose a pharmaceutical composition comprising such active ingredient(s) for use in e.g., treating such a specific condition, in e.g., treating such specific condition in such selected group of patients, e.g., for treating such a specific condition by using such a specific dosage regimen, etc. The uses as mentioned above for the second embodiment thus also disclose pharmaceutical compositions comprising the active ingredients as mentioned for use in the treatment of a hepatitis viral infection in a human or animal individual who does not respond or is refractory to treatment with at least one pharmaceutically active agent for treatment of a viral hepatitis infection.

In a fourth embodiment, the invention thus relates to a pharmaceutical composition for use in the treatment of a hepatitis viral infection in a human or animal individual who does not respond or is refractory to treatment with at least one pharmaceutically active agent for treatment of a viral hepatitis infection, comprising at least one proteasome inhibitor being selected from PS-341 and S-2209 and at least one first pharmaceutically active agent for treatment of a viral hepatitis infection, wherein said at least one first pharmaceutically active agent for treatment of a viral hepatitis infection is pegylated interferon-alpha, and said at least one second pharmaceutically active agent for treatment of a viral hepatitis infection, where present, is ribavirin.

In a fifth embodiment, the invention relates to a method of treating a hepatitis viral infection in a human or animal individual who does not respond or is refractory to treatment with at least one pharmaceutically active agent for treatment of a viral hepatitis infection, comprising the step of administering to such individual at least one kit of pharmaceutical compositions as provided herein, or at least one pharmaceutical composition as provided herein.

In a sixth embodiment, the invention relates to a combination of at least one proteasome inhibitor, at least one first pharmaceutically active agent for treatment of a viral hepatitis infection, and optionally at least one second pharmaceutically active agent for treatment of a viral hepatitis infection, for the treatment of a hepatitis viral infection in a human or animal individual who does not respond or is refractory to treatment with at least one pharmaceutically active agent for treatment of a viral hepatitis infection, essentially as provided by the first embodiment's kits of pharmaceutical compositions described herein before, or the second embodiment's uses described herein before.

In a seventh embodiment, the invention relates to the novel kits, compositions, methods and uses essentially as provided by the first through sixth embodiment described herein before.

In an eighth embodiment, the invention relates to a kit for the treatment of a hepatitis viral infection in a human or animal individual, comprising: (a) at least one first pharmaceutical container containing a composition comprising at least one proteasome inhibitor; (b) at least one second pharmaceutical container containing a composition comprising at least one first pharmaceutically active agent for treatment of a viral hepatitis infection, and optionally at least one second pharmaceutically active agent for treatment of a viral hepatitis infection, in said at least one second or in at least one third pharmaceutical container; and (c) instructions in paper or electronic form advising the user to first administer the composition of the at least one first pharmaceutical container and administer the composition or at least one second and, where present, at least one third pharmaceutical container subsequently after a delay, or advising the user to first administer composition of the at least one second and, where present, at least one third pharmaceutical container and administer the compositions of the at least one first pharmaceutical container subsequently after a delay.

In one embodiment, the invention relates to a kit, wherein said kit comprises a composition containing said at least one second pharmaceutically active agent for treatment of a viral hepatitis infection contained in said at least one second or in at least one third pharmaceutical container.

In one embodiment, the invention relates to a kit, wherein: said at least one proteasome inhibitor is selected from the group comprising proteasome-specific inhibitors; said at least one first pharmaceutically active agent for treatment of a viral hepatitis infection is selected from one of (A) the group comprising agents which support or assist the body's natural response in dealing with viral infections, or (B) the group comprising agents which interfere with the function of a viral target; and said at least one second pharmaceutically active agent for treatment of a viral hepatitis infection, where present, is selected from (B) if the first pharmaceutically active agent for treatment of a viral hepatitis infection is selected from (A), or selected from (A) if the first pharmaceutically active agent for treatment of a viral hepatitis infection is selected from (B)

In one embodiment, the invention relates to a kit, wherein: said at least one proteasome inhibitor is selected from the group comprising proteasome-specific inhibitors; said group (A) comprising agents which support or assist the body's natural response in dealing with viral infections comprises interferons including their derivatives, interleukins, steroids, immunomodulators, immunosuppressants, and inhibitors of assisted protein folding; and said group (B) comprising agents which interfere with the function of a viral target comprises inhibitors of HCV NS3/4A protease, HCV NS4B protein, HCV NS5A protein, HCV NS5B polymerase and HCV envelope proteins.

In one embodiment, the invention relates to a kit, wherein: said group (A) comprising agents which support or assist the body's natural response in dealing with viral infections comprises interferons including their derivatives; and said group (B) comprising agents which interfere with the function of a viral target comprises nucleoside or nucleotide analogs.

In one embodiment, the invention relates to a kit, wherein: said group (A) comprising agents which support or assist the body's natural response in dealing with viral infections consists of: interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD-101, IMO-2125, GI-5005 (Tarmogen), IC41, PF-04878691, TG 4040 and the glycosylated, pegylated or hesylated forms thereof; and said group (B) comprising agents which interfere with the function of a viral target consists of: lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222, PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132).

In one embodiment, the invention relates to a kit, wherein: said group (A) comprising agents which support or assist the body's natural response in dealing with viral infections consists of: pegylated forms of interferon-alpha, beta, gamma or omega; and said group (B) comprising agents which interfere with the function of a viral target consists of: ribavirin.

In one embodiment, the invention relates to a kit, wherein said at least one proteasome inhibitor is selected from the group comprising: peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (also known as aclarubicine), celastrol, withaferin A, Gliotoxin, epipolythiodioxo-piperazines, green tea polyphenolic catechins such as (−)-epigallocatechin-3-gallate, Disulfuram, acridine derivatives including tetra-acridine derivatives with betulinic acid such as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, its endocyclic oxindole-phenyl clamp (BIA-1a) and endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal, its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer “PS-293”, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209 and its structural analogs Compound 1-6 and Compound 8.

In one embodiment, the invention relates to a kit, wherein said at least one proteasome inhibitor is PS-273, PS-341, PS-519, or S-2209.

In one embodiment, the invention relates to a kit, wherein said at least one proteasome inhibitor is PS-341, wherein said interferon is pegylated interferon alpha, and wherein said nucleoside analog is ribavirin.

In one embodiment, the invention relates to a kit, wherein said at least one proteasome inhibitor is S-2209.

In one embodiment, the invention relates to a kit, wherein said at least one proteasome inhibitor is S-2209, wherein said interferon is pegylated interferon alpha, and wherein said nucleoside analog is ribavirin.

In one embodiment, the invention relates to a kit, further comprising instructions in paper or electronic form advising the user to administer at least one of the at least one first and/or, where present, at least one second pharmaceutically active agent for treatment of a viral hepatitis infection for not more than about 50% of the duration otherwise recommended for the treatment of the hepatitis viral infection with the respective pharmaceutically active agent for treatment of a viral hepatitis infection and/or to administer not more than about 66% of the dose recommended for the treatment of the hepatitis viral infection with the respective pharmaceutically active agent for treatment of a viral hepatitis infection.

In one embodiment, the invention relates to a kit, further comprising instructions in paper or electronic form advising the user to apply the kit for the treatment of a human or animal individual ineligible for or unwilling to undergo treatment with at least one pharmaceutically active agent for treatment of a viral hepatitis infection.

In one embodiment, the invention relates to a kit, further comprising instructions in paper or electronic form advising the user to apply the kit for the treatment of a human or animal individual who does not respond or is refractory to treatment with at least one pharmaceutically active agent for treatment of a viral hepatitis infection.

In one embodiment, the invention relates to a kit, further comprising instructions in paper or electronic form advising the user to first administer the at least one first pharmaceutical composition and administer the at least one second and, where present, at least one third pharmaceutical composition subsequently with delay.

In one embodiment, the invention relates to a kit, wherein the instructions further advise the user on a specified time period for said delay.

In one embodiment, the invention relates to a kit, wherein said specified time period is about 1 day, about 2 days, about 2 to 4 days, about 4 to 6 days, about 1 week, about 1 to 2 weeks, about 2 to 3 weeks, about 2 to 4 weeks, about 2 to 5 weeks, about 2 to 6 weeks, about 2 to 7 weeks, about 2 to 8 weeks, about 3 to 4 weeks, about 3 to 5 weeks, about 3 to 6 weeks, about 3 to 7 weeks, about 3 to 8 weeks, about 4 to 5 weeks, about 4 to 6 weeks, about 4 to 7 weeks, about 4 to 8 weeks, about 4 to 10 weeks, about 4 to 12 weeks, about 6 to 8 weeks, about 6 to 10 weeks, or about 6 to 12 weeks; or more than about 2 weeks, more than about 3 weeks, more than about 4 weeks, more than about 5 weeks, more than about 6 weeks, more than about 7 weeks, more than about 8 weeks, more than about 10 weeks, or more than about 12 weeks and/or less than about 4 weeks, less than about 5 weeks, more less 6 weeks, less than about 7 weeks, less than about 8 weeks, less than about 10 weeks, or less than about 12 weeks, or any other combination not yet expressly given above of these upper and lower limits.

In one embodiment, the invention relates to a kit, wherein said specified time period is more than about 2 weeks, and less than about 8 weeks. In another embodiment, said specified time period is 2-8 weeks.

In one embodiment, the invention relates to a kit, further comprising instructions in paper or electronic form advising the user to perform one to two rounds of administrations, each round consisting of 3 to 10 administrations, of the at least one first pharmaceutical composition comprising the at least one proteasome inhibitor.

In one embodiment, the invention relates to a kit, wherein the hepatitis viral infection is an infection with Hepatitis C virus.

In a ninth embodiment, the invention relates to a use of at least one proteasome inhibitor and at least one first pharmaceutically active agent for treatment of a viral hepatitis infection, and optionally at least one second pharmaceutically active agent for treatment of a viral hepatitis infection, in the manufacture of a pharmaceutical composition for the treatment of a hepatitis viral infection in a human or animal individual, wherein said pharmaceutical composition is provided in a manner such that the at least one first proteasome inhibitor is administered first, and the at least one first pharmaceutically active agent for treatment of a viral hepatitis infection and, where present, the at least one second pharmaceutically active agent for treatment of a viral hepatitis infection are administered subsequently with delay, or such that the at least one first pharmaceutically active agent for treatment of a viral hepatitis infection and, where present, the at least one second pharmaceutically active agent for treatment of a viral hepatitis infection are administered first, and the at least one proteasome inhibitor is administered subsequently with delay.

In one embodiment, the invention relates to a use, wherein said at least one second pharmaceutically active agent for treatment of a viral hepatitis infection is used in the manufacture of said pharmaceutical composition.

In one embodiment, the invention relates to a use, wherein said at least one proteasome inhibitor is selected from the group comprising proteasome-specific inhibitors; said at least one first pharmaceutically active agent for treatment of a viral hepatitis infection is selected from one of (A) the group comprising agents which support or assist the body's natural response in dealing with viral infections, or (B) the group comprising agents which interfere with the function of a viral target; and said at least one second pharmaceutically active agent for treatment of a viral hepatitis infection, where present, is selected from (B) if the first pharmaceutically active agent for treatment of a viral hepatitis infection is selected from (A), or selected from (A) if the first pharmaceutically active agent for treatment of a viral hepatitis infection is selected from (B).

In one embodiment, the invention relates to a use, wherein said at least one proteasome inhibitor is selected from the group comprising proteasome-specific inhibitors; said group (A) comprising agents which support or assist the body's natural response in dealing with viral infections comprises interferons including their derivatives, interleukins, steroids, immunomodulators, immunosuppressants, and inhibitors of assisted protein folding; and said group (B) comprising agents which interfere with the function of a viral target comprises inhibitors of HCV NS3/4A protease, inhibitors of HCV NS4B protein, inhibitors of HCV NS5A protein, inhibitors of HCV NS5B polymerase and inhibitors of HCV envelope proteins.

In one embodiment, the invention relates to a use, wherein said group (A) comprising agents which support or assist the body's natural response in dealing with viral infections comprises interferons including their derivatives; and said group (B) comprising agents which interfere with the function of a viral target comprises nucleoside or nucleotide analogs.

In one embodiment, the invention relates to a use, wherein said group (A) comprising agents which support or assist the body's natural response in dealing with viral infections consists of: interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD-101, IMO-2125, GI-5005 (Tarmogen), IC41, PF-04878691, TG 4040 and the glycosylated, pegylated or hesylated forms thereof; and said group (B) comprising agents which interfere with the function of a viral target consists of: lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222, PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132).

In one embodiment, the invention relates to a use, wherein said group (A) comprising agents which support or assist the body's natural response in dealing with viral infections consists of pegylated forms of interferon-alpha, beta, gamma or omega; and said group (B) comprising agents which interfere with the function of a viral target consists of: ribavirin.

In one embodiment, the invention relates to a use, wherein said at least one proteasome inhibitor is selected from the group comprising: peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (also known as aclarubicine), celastrol, withaferin A, Gliotoxin, epipolythiodioxo-piperazines, green tea polyphenolic catechins such as (−)-epigallocatechin-3-gallate, Disulfuram, acridine derivatives including tetra-acridine derivatives with betulinic acid such as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, its endocyclic oxindole-phenyl clamp (BIA-1a) and endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal, its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer “PS-293”, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209 and its structural analogs Compound 1-6 and Compound 8.

In one embodiment, the invention relates to a use, wherein said at least one proteasome inhibitor is PS-273, PS-341, PS-519, or S-2209.

In one embodiment, the invention relates to a use, wherein said at least one proteasome inhibitor is PS-341, wherein said interferon is pegylated interferon alpha, and wherein said nucleoside analog is ribavirin.

In one embodiment, the invention relates to a use, wherein said at least one proteasome inhibitor is S-2209.

In one embodiment, the invention relates to a use, wherein said at least one proteasome inhibitor is S-2209, wherein said interferon is pegylated interferon alpha, and wherein said nucleoside analog is ribavirin.

In one embodiment, the invention relates to a use, wherein at least one of the said at least one first and/or, where present, at least one second pharmaceutically active agent for treatment of a viral hepatitis infection is provided such that the at least one first and/or, where present, at least one second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the hepatitis viral infection with the respective pharmaceutically active agent for treatment of a viral hepatitis infection is administered.

In one embodiment, the invention relates to a use, wherein the pharmaceutical composition is provided for the treatment of a human or animal individual ineligible for or unwilling to undergo treatment with at least one pharmaceutically active agent for treatment of a viral hepatitis infection.

In one embodiment, the invention relates to a use, wherein the pharmaceutical composition is provided for the treatment of a hepatitis viral infection in a human or animal individual who does not respond or is refractory to treatment with at least one pharmaceutically active agent for treatment of a viral hepatitis infection.

In one embodiment, the invention relates to a use, wherein the pharmaceutical composition is provided in a manner such that the at least one first proteasome inhibitor is administered first, and the at least one first pharmaceutically active agent for treatment of a viral hepatitis infection and, where present, the at least one second pharmaceutically active agent for treatment of a viral hepatitis infection are administered subsequently after a delay.

In one embodiment, the invention relates to a use, wherein the pharmaceutical composition is provided in a manner such that the at least one first pharmaceutically active agent for treatment of a viral hepatitis infection and, where present, the at least one second pharmaceutically active agent for treatment of a viral hepatitis infection are administered first, and the at least one proteasome inhibitor is administered subsequently with delay.

In one embodiment, the invention relates to a use, wherein said delay is a specified time period.

In one embodiment, the invention relates to a use, wherein said specified time period is about 1 day, about 2 days, about 2 to 4 days, about 4 to 6 days, about 1 week, about 1 to 2 weeks, about 2 to 3 weeks, about 2 to 4 weeks, about 2 to 5 weeks, about 2 to 6 weeks, about 2 to 7 weeks, about 2 to 8 weeks, about 3 to 4 weeks, about 3 to 5 weeks, about 3 to 6 weeks, about 3 to 7 weeks, about 3 to 8 weeks, about 4 to 5 weeks, about 4 to 6 weeks, about 4 to 7 weeks, about 4 to 8 weeks, about 4 to 10 weeks, about 4 to 12 weeks, about 6 to 8 weeks, about 6 to 10 weeks, or about 6 to 12 weeks; or more than about 2 weeks, more than about 3 weeks, more than about 4 weeks, more than about 5 weeks, more than about 6 weeks, more than about 7 weeks, more than about 8 weeks, more than about 10 weeks, or more than about 12 weeks and/or less than about 4 weeks, less than about 5 weeks, more less 6 weeks, less than about 7 weeks, less than about 8 weeks, less than about 10 weeks, or less than about 12 weeks, or any other combination not yet expressly given above of these upper and lower limits.

In one embodiment, the invention relates to a use, wherein said specified time period is more than about 2 weeks, but less than about 8 weeks.

In one embodiment, the invention relates to a use, wherein the pharmaceutical composition is provided in a manner such that one to two rounds of administrations, each round consisting of 3 to 10 administrations, of the at least one proteasome inhibitor are performed.

In one embodiment, the invention relates to a use, wherein the hepatitis viral infection is an infection with Hepatitis C virus.

In a tenth embodiment, the invention relates to pharmaceutical compositions essentially as provided by the ninth embodiment's uses described herein.

If in the context of the present invention it is stated that the invention relates to a use of an active ingredient such as e.g., at least one proteasome inhibitor and e.g., at least one first pharmaceutically active agent for treatment of a viral hepatitis infection in the manufacture of a pharmaceutical composition e.g., for treating a specific condition, e.g., for treating a specific condition in a selected group of patients, e.g., for using a specific dosage regimen in the treatment of a specific condition, etc., then such statements are meant to also disclose a pharmaceutical composition comprising such active ingredient(s) for use in e.g., treating such a specific condition, in e.g., treating such specific condition in such selected group of patients, e.g., for treating such a specific condition by using such a specific dosage regimen, etc. The uses as mentioned above for the ninth embodiment thus also disclose pharmaceutical compositions comprising the active ingredients as mentioned for use in the treatment of a hepatitis viral infection in a human or animal individual by administering the pharmaceutical composition in a manner such that the at least one first proteasome inhibitor is administered first, and the at least one first pharmaceutically active agent for treatment of a viral hepatitis infection and, where present, the at least one second pharmaceutically active agent for treatment of a viral hepatitis infection are administered subsequently with delay, or such that the at least one first pharmaceutically active agent for treatment of a viral hepatitis infection and, where present, the at least one second pharmaceutically active agent for treatment of a viral hepatitis infection are administered first, and the at least one proteasome inhibitor is administered subsequently with delay.

In an eleventh embodiment, the invention relates to a pharmaceutical composition comprising at least one proteasome inhibitor being selected from PS-341 and S-2209 and at least one first pharmaceutically active agent for use in the treatment of viral hepatitis infections by administering at least one proteasome inhibitor first to the patient and the at least one first pharmaceutically active agent for treatment of a viral hepatitis infection subsequently to the patient with delay.

In a twelfth embodiment, the invention relates to a method of treating a hepatitis viral infection in a human or animal individual comprising the step of administering to such individual at least one kit of pharmaceutical compositions essentially as provided by the eighth embodiment's kits of pharmaceutical compositions described herein, or at least one pharmaceutical composition essentially as provided by the ninth embodiment's uses described herein.

In a thirteenth embodiment, the invention relates to a combination of at least one proteasome inhibitor, at least one first pharmaceutically active agent for treatment of a viral hepatitis infection, and optionally at least one second pharmaceutically active agent for treatment of a viral hepatitis infection, for the treatment of a hepatitis viral infection in a human or animal individual, essentially as provided by the eighth embodiment's kits of pharmaceutical compositions described herein before, or the ninth embodiment's uses described herein before.

In a fourteenth embodiment, the invention relates to the novel kits, compositions, methods and use essentially as provided by the eighth through thirteenth embodiment described herein before.

In a fifteenth embodiment, the invention relates to a kit of pharmaceutical compositions for the treatment of a hepatitis viral infection in a human or animal individual ineligible for or unwilling to undergo treatment with at least one pharmaceutically active agent for treatment of a viral hepatitis infection, comprising: at least one first pharmaceutical container comprising a pharmaceutical composition comprising at least one proteasome inhibitor; at least one second pharmaceutical container comprising a pharmaceutical composition comprising at least one first pharmaceutically active agent for treatment of a viral hepatitis infection, and optionally at least one second pharmaceutically active agent for treatment of a viral hepatitis infection, comprised in a composition of said at least one second or in at least one third pharmaceutical containers; instructions in paper or electronic form advising the user to administer at least one of the at least one second and, where present, third pharmaceutical compositions for a reduced duration compared to the duration otherwise recommended for the treatment of the hepatitis viral infection with the respective pharmaceutically active agent for treatment of a viral hepatitis infection and/or to administer the compositions of at least one of the at least one second and, where present, third pharmaceutical container such that a reduced dose compared to the dose recommended for the treatment of the hepatitis viral infection with the respective pharmaceutically active agent for treatment of a viral hepatitis infection is administered.

In one embodiment, the invention relates to a kit, wherein said kit comprises said at least one second pharmaceutically active agent for treatment of a viral hepatitis infection contained in the composition of said at least one second or in at least one third pharmaceutical container.

In one embodiment, the invention relates to a kit, wherein: said at least one proteasome inhibitor is selected from the group comprising proteasome-specific inhibitors; said at least one first pharmaceutically active agent for treatment of a viral hepatitis infection is selected from one of (A) the group comprising agents which support or assist the body's natural response in dealing with viral infections, or (B) the group comprising agents which interfere with the function of a viral target; and said at least one second pharmaceutically active agent for treatment of a viral hepatitis infection, where present, is selected from selected from (B) if the first pharmaceutically active agent for treatment of a viral hepatitis infection is selected from (A), or selected from (A) if the first pharmaceutically active agent for treatment of a viral hepatitis infection is selected from (B).

In one embodiment, the invention relates to a kit, wherein: said at least one proteasome inhibitor is selected from the group comprising proteasome-specific inhibitors; said group (A) comprising agents which support or assist the body's natural response in dealing with viral infections comprises interferons including their derivatives, interleukins, steroids, immunomodulators, immunosuppressants, and inhibitors of assisted protein folding; and said group (B) comprising agents which interfere with the function of a viral target comprises inhibitors of HCV NS3/4A protease, HCV NS4B protein, HCV NS5A protein, HCV NS5B polymerase and HCV envelope proteins.

In one embodiment, the invention relates to a kit, wherein: said group (A) comprising agents which support or assist the body's natural response in dealing with viral infections comprises interferons including their derivatives; and said group (B) comprising agents which interfere with the function of a viral target comprises nucleoside or nucleotide analogs.

In one embodiment, the invention relates to a kit, wherein: said group (A) comprising agents which support or assist the body's natural response in dealing with viral infections consists of: interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD-101, IMO-2125, GI-5005 (Tarmogen), IC41, PF-04878691, TG 4040 and the glycosylated, pegylated or hesylated forms thereof; and said group (B) comprising agents which interfere with the function of a viral target consists of: lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222, PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132).

In one embodiment, the invention relates to a kit, wherein: said group (A) comprising agents which support or assist the body's natural response in dealing with viral infections consists of: pegylated forms of interferon-alpha, beta, gamma or omega; and said group (B) comprising agents which interfere with the function of a viral target consists of: ribavirin.

In one embodiment, the invention relates to a kit, wherein said at least one proteasome inhibitor is selected from the group comprising: peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (also known as aclarubicine), celastrol, withaferin A, Gliotoxin, epipolythiodioxo-piperazines, green tea polyphenolic catechins such as (−)-epigallocatechin-3-gallate, Disulfuram, acridine derivatives including tetra-acridine derivatives with betulinic acid such as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, its endocyclic oxindole-phenyl clamp (BIA-1a) and endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal, its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer “PS-293”, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209 and its structural analogs Compound 1-6 and Compound 8.

In one embodiment, the invention relates to a kit, wherein said at least one proteasome inhibitor is PS-273, PS-341, PS-519, or S-2209.

In one embodiment, the invention relates to a kit, wherein said at least one proteasome inhibitor is PS-341, wherein said interferon is pegylated interferon alpha, and wherein said nucleoside analog is ribavirin.

In one embodiment, the invention relates to a kit, wherein said at least one proteasome inhibitor is S-2209.

In one embodiment, the invention relates to a kit, wherein said at least one proteasome inhibitor is S-2209, wherein said interferon is pegylated interferon alpha, and wherein said nucleoside analog is ribavirin.

In one embodiment, the invention relates to a kit, further comprising instructions in paper or electronic form advising the user to administer at least one of the at least one first and/or, where present, at least one second pharmaceutically active agent for treatment of a viral hepatitis infection for not more than about 95%, 90% 85%, 80%, 75% 70%, 66%, 50%, 40%, 33%, 25% 15% or 10% of the duration otherwise recommended for the treatment of the hepatitis viral infection with the respective pharmaceutically active agent for treatment of a viral hepatitis infection and/or to administer not more than about 95%, 90% 85%, 80%, 75% 70%, 66%, 50%, 40%, 33%, 25% 15% or 10% of the dose recommended for the treatment of the hepatitis viral infection with the respective pharmaceutically active agent for treatment of a viral hepatitis infection.

In one embodiment, the invention relates to a kit, wherein the user is advised to administer at least one of the at least one first and/or, where present, at least one second pharmaceutically active agent for treatment of a viral hepatitis infection for not more than about 50% of the duration otherwise recommended for the treatment of the hepatitis viral infection with the respective pharmaceutically active agent for treatment of a viral hepatitis infection and/or to administer not more than about 66% of the dose recommended for the treatment of the hepatitis viral infection with the respective pharmaceutically active agent for treatment of a viral hepatitis infection.

In one embodiment, the invention relates to a kit, further comprising instructions in paper or electronic form advising the user to apply the kit for the treatment of a human or animal individual who does not respond or is refractory to treatment with at least one pharmaceutically active agent for treatment of a viral hepatitis infection.

In one embodiment, the invention relates to a kit, further comprising instructions in paper or electronic form advising the user to administer the at least one first pharmaceutical composition and the at least one second and, where present, at least one third pharmaceutical composition concurrently.

In one embodiment, the invention relates to a kit, further comprising instructions in paper or electronic form advising the user to first administer the at least one first pharmaceutical composition and administer the at least one second and, where present, at least one third pharmaceutical composition subsequently with delay.

In one embodiment, the invention relates to a kit, further comprising instructions in paper or electronic form advising the user to first administer the at least one second and, where present, at least one third pharmaceutical composition, and administer the at least one first pharmaceutical composition subsequently with delay.

In one embodiment, the invention relates to a kit, wherein the instructions further advise the user on a specified time period for said delay.

In one embodiment, the invention relates to a kit, wherein said specified time period is more than about 2 weeks, and less than about 8 weeks.

In one embodiment, the invention relates to a kit, further comprising instructions in paper or electronic form advising the user to perform one to two rounds of administrations, each round consisting of 3 to 10 administrations, of the at least one first pharmaceutical composition comprising the at least one proteasome inhibitor.

In one embodiment, the invention relates to a kit, wherein the hepatitis viral infection is an infection with Hepatitis C virus.

In a sixteenth embodiment, the invention relates to a use of at least one proteasome inhibitor and at least one first pharmaceutically active agent for treatment of a viral hepatitis infection, and optionally at least one second pharmaceutically active agent for treatment of a viral hepatitis infection, in the manufacture of a pharmaceutical composition for the treatment of a hepatitis viral infection in a human or animal individual ineligible for or unwilling to undergo treatment with at least one pharmaceutically active agent for treatment of a viral hepatitis infection, wherein said at least one first and/or, where present, at least one second pharmaceutically active agent for treatment of a viral hepatitis infection is provided such that the at least one first and/or, where present, at least one second pharmaceutically active agent for treatment of a viral hepatitis infection is administered for a reduced duration compared to the duration otherwise recommended for the treatment of the hepatitis viral infection with the respective pharmaceutically active agent for treatment of a viral hepatitis infection and/or such that a reduced dose compared to the dose recommended for the treatment of the hepatitis viral infection with the respective pharmaceutically active agent for treatment of a viral hepatitis infection is administered.

In one embodiment, the invention relates to a use, wherein said at least one second pharmaceutically active agent for treatment of a viral hepatitis infection is used in the manufacture of said pharmaceutical composition.

In one embodiment, the invention relates to a use, wherein said at least one proteasome inhibitor is selected from the group comprising proteasome-specific inhibitors; said at least one first pharmaceutically active agent for treatment of a viral hepatitis infection is selected from one of (A) the group comprising agents which support or assist the body's natural response in dealing with viral infections, or (B) the group comprising agents which interfere with the function of a viral target; and said at least one second pharmaceutically active agent for treatment of a viral hepatitis infection, where present, is selected from (B) if the first pharmaceutically active agent for treatment of a viral hepatitis infection is selected from (A), or selected from (A) if the first pharmaceutically active agent for treatment of a viral hepatitis infection is selected from (B).

In one embodiment, the invention relates to a use, wherein said at least one proteasome inhibitor is selected from the group comprising proteasome-specific inhibitors; said group (A) comprising agents which support or assist the body's natural response in dealing with viral infections comprises interferons including their derivatives, interleukins, steroids, immunomodulators, immunosuppressants, and inhibitors of assisted protein folding; and said group (B) comprising agents which interfere with the function of a viral target comprises inhibitors of HCV NS3/4A protease, HCV NS4B protein, HCV NS5A protein, HCV NS5B polymerase and HCV envelope proteins.

In one embodiment, the invention relates to a use, wherein said group (A) comprising agents which support or assist the body's natural response in dealing with viral infections comprises interferons including their derivatives; and said group (B) comprising agents which interfere with the function of a viral target comprises nucleoside or nucleotide analogs.

In one embodiment, the invention relates to a use, wherein said group (A) comprising agents which support or assist the body's natural response in dealing with viral infections consists of: interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD-101, IMO-2125, GI-5005 (Tarmogen), IC41, PF-04878691, TG 4040 and the glycosylated, pegylated or hesylated forms thereof; and said group (B) comprising agents which interfere with the function of a viral target consists of: lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222, PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132).

In one embodiment, the invention relates to a use, wherein said group (A) comprising agents which support or assist the body's natural response in dealing with viral infections consists of: pegylated forms of interferon-alpha, beta, gamma or omega; and said group (B) comprising agents which interfere with the function of a viral target consists of: ribavirin.

In one embodiment, the invention relates to a use, wherein said at least one proteasome inhibitor is selected from the group comprising: peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (also known as aclarubicine), celastrol, withaferin A, Gliotoxin, epipolythiodioxo-piperazines, green tea polyphenolic catechins such as (−)-epigallocatechin-3-gallate, Disulfuram, acridine derivatives including tetra-acridine derivatives with betulinic acid such as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, its endocyclic oxindole-phenyl clamp (BIA-1a) and endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal, its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer “PS-293”, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209 and its structural analogs Compound 1-6 and Compound 8.

In one embodiment, the invention relates to a use, wherein said at least one proteasome inhibitor is PS-273, PS-341, PS-519, or S-2209.

In one embodiment, the invention relates to a use, wherein said at least one proteasome inhibitor is PS-341, wherein said interferon is pegylated interferon alpha, and wherein said nucleoside analog is ribavirin.

In one embodiment, the invention relates to a use, wherein said at least one proteasome inhibitor is S-2209.

In one embodiment, the invention relates to a use, wherein said at least one proteasome inhibitor is S-2209, wherein said interferon is pegylated interferon alpha, and wherein said nucleoside analog is ribavirin.

In one embodiment, the invention relates to a use, wherein at least one of the said at least one first and/or, where present, at least one second pharmaceutically active agent for treatment of a viral hepatitis infection is provided such that the at least one first and/or, where present, at least one second pharmaceutically active agent for treatment of a viral hepatitis infection is administered for not more than about 95%, 90% 85%, 80%, 75% 70%, 66%, 50%, 40%, 33%, 25% 15% or 10% of the duration otherwise recommended for the treatment of the hepatitis viral infection with the respective pharmaceutically active agent for treatment of a viral hepatitis infection and/or such that not more than about 95%, 90% 85%, 80%, 75% 70%, 66%, 50%, 40%, 33%, 25% 15% or 10% of the dose recommended for the treatment of the hepatitis viral infection with the respective pharmaceutically active agent for treatment of a viral hepatitis infection is administered.

In one embodiment, the invention relates to a use, wherein at least one of said at least one first and/or, where present, at least one second pharmaceutically active agent for treatment of a viral hepatitis infection is provided such that the at least one first and/or, where present, at least one second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the hepatitis viral infection with the respective pharmaceutically active agent for treatment of a viral hepatitis infection is administered.

In one embodiment, the invention relates to a use, wherein the pharmaceutical composition is provided in a manner such that the treatment of a hepatitis viral infection in a human or animal individual who does not respond or is refractory to treatment with at least one pharmaceutically active agent for treatment of a viral hepatitis infection is enabled.

In one embodiment, the invention relates to a use, wherein the pharmaceutical composition is provided in a manner such that the proteasome inhibitor, the first pharmaceutically active agent for treatment of a viral hepatitis infection and, where present, the second pharmaceutically active agent for treatment of a viral hepatitis infection are administered concurrently.

In one embodiment, the invention relates to a use, wherein the pharmaceutical composition is provided in a manner such that the at least one first proteasome inhibitor is administered first, and the at least one first pharmaceutically active agent for treatment of a viral hepatitis infection and, where present, the at least one second pharmaceutically active agent for treatment of a viral hepatitis infection are administered subsequently with delay.

In one embodiment, the invention relates to a use, wherein the pharmaceutical composition is provided in a manner such that the at least one first pharmaceutically active agent for treatment of a viral hepatitis infection and, where present, the at least one second pharmaceutically active agent for treatment of a viral hepatitis infection are administered first, and the at least one proteasome inhibitor is administered subsequently with delay.

In one embodiment, the invention relates to a use, wherein said delay is a specified time period.

In one embodiment, the invention relates to a use, wherein said specified time period is more than about 2 weeks, but less than about 8 weeks.

In one embodiment, the invention relates to a use, wherein the pharmaceutical composition is provided in a manner such that one to two rounds of administrations, each round consisting of 3 to 10 administrations, of the at least one proteasome inhibitor are performed.

In one embodiment, the invention relates to a use, wherein the hepatitis viral infection is an infection with Hepatitis C virus.

In a seventeenth embodiment, the invention relates to a pharmaceutical composition essentially as provided by the uses of the sixteenth embodiment described herein before.

If in the context of the present invention it is stated that the invention relates to a use of an active ingredient such as e.g., at least one proteasome inhibitor and e.g., at least one first pharmaceutically active agent for treatment of a viral hepatitis infection in the manufacture of a pharmaceutical composition e.g., for treating a specific condition, e.g., for treating a specific condition in a selected group of patients, e.g., for using a specific dosage regimen in the treatment of a specific condition, etc., then such statements are meant to also disclose a pharmaceutical composition comprising such active ingredient(s) for use in e.g., treating such a specific condition, in e.g., treating such specific condition in such selected group of patients, e.g., for treating such a specific condition by using such a specific dosage regimen, etc. The uses as mentioned above for the sixteenth embodiment thus also disclose pharmaceutical compositions comprising the active ingredients as mentioned for use in the treatment of a hepatitis viral infection in a human or animal individual ineligible for or unwilling to undergo treatment with at least one pharmaceutically active agent for treatment of a viral hepatitis infection.

In an eighteenth embodiment, the invention relates to a pharmaceutical composition for use in the treatment of a hepatitis viral infection in a human or animal individual ineligible for or unwilling to undergo treatment with at least one pharmaceutically active agent for treatment of a viral hepatitis infection, comprising at least one proteasome inhibitor being selected from PS-341 and S-2209, at least one first pharmaceutically active agent for treatment of a viral hepatitis infection, and, optionally, at least one second pharmaceutically active agent for treatment of a viral hepatitis infection, wherein said at least one first and/or, where present, at least one second pharmaceutically active agent for treatment of a viral hepatitis infection is provided such that the at least one first and/or, where present, at least one second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the hepatitis viral infection with the respective pharmaceutically active agent for treatment of a viral hepatitis infection is administered.

In an nineteenth embodiment, the invention relates to a method of treating a hepatitis viral infection in a human or animal individual ineligible for or unwilling to undergo treatment with at least one pharmaceutically active agent for treatment of a viral hepatitis infection, comprising the step of administering to such individual at least one kit of pharmaceutical compositions essentially as provided by the fifteenth embodiment's kits of pharmaceutical compositions described herein, or at least one pharmaceutical composition essentially as provided by the sixteenth embodiment's uses described herein before.

In a twentieth embodiment, the invention relates to a combination of at least one proteasome inhibitor, at least one first pharmaceutically active agent for treatment of a viral hepatitis infection, and optionally at least one second pharmaceutically active agent for treatment of a viral hepatitis infection, for the treatment of a hepatitis viral infection in a human or animal individual ineligible for or unwilling to undergo treatment with at least one pharmaceutically active agent for treatment of a viral hepatitis infection, essentially as provided by the fifteenth embodiment's kits of pharmaceutical compositions described herein, or the seventeenth embodiment's uses described herein before.

In a twenty-first embodiment, the invention relates to the novel kits, compositions, methods and uses essentially as provided by the fifteenth through twentieth embodiments described herein before.

In one aspect, the present invention thus relates to a pharmaceutical composition comprising at least one proteasome inhibitor, at least one first pharmaceutically active agent for treatment of a viral hepatitis infection, and optionally at least one second pharmaceutically active agent for treatment of a viral hepatitis infection. Said at least one first pharmaceutically active agent for treatment of a viral hepatitis infection may be selected from one of (A) the group comprising agents which support or assist the body's natural response in dealing with viral infections, or (B) the group comprising agents which interfere with the function of a viral target; and said at least one second pharmaceutically active agent for treatment of a viral hepatitis infection, where present, is selected from (B) if the first pharmaceutically active agent for treatment of a viral hepatitis infection is selected from (A), or selected from (A) if the first pharmaceutically active agent for treatment of a viral hepatitis infection is selected from (B). Such pharmaceutical compositions can comprise combinations of specific active ingredients as they are specifically mentioned in the first embodiment in the context of kits.

In one embodiment, the proteasome-specific inhibitors of such pharmaceutical compositions may thus be selected from the group consisting of PS-273, PS-341, PS-519, or S-2209. Group (A) comprising agents which support or assist the body's natural response in dealing with viral infections of such pharmaceutical compositions may comprise interferons including their derivatives. Group (B) comprising agents which interfere with the function of a viral target of such pharmaceutical compositions may comprise nucleoside or nucleotide analogs.

In some embodiments, the proteasome inhibitors of such pharmaceutical compositions can be selected from PS-273, PS-341, PS-519, or S-2209. Agents of group (A) of such pharmaceutical compositions may be selected from the group consisting of interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD-101, IMO-2125, GI-5005 (Tarmogen), IC41, PF-04878691, TG 4040 and the glycosylated, pegylated or hesylated forms thereof. Agents of group (B) of such pharmaceutical compositions may be selected from the group consisting of lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222, PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132).

In some embodiments, the proteasome inhibitors of such pharmaceutical compositions may be selected from PS-273, PS-341, PS-519, or S-2209. The agents of said group (A) of such pharmaceutical compositions may be selected from pegylated forms of interferon-alpha, beta, gamma or omega; and the agents of said group (B) of such pharmaceutical compositions may be selected from ribavirin. The pharmaceutical compositions may thus comprise PS-341, pegylated interferon alpha, and ribavirin. The pharmaceutical compositions may thus also comprise S-2209, pegylated interferon alpha, and ribavirin.

In a twenty-second embodiment, the present invention relates to a kit of pharmaceutical compositions comprising: a) at least one first pharmaceutical container containing a pharmaceutical at least one proteasome inhibitor; b) at least one second pharmaceutical container containing a pharmaceutical at least one first pharmaceutically active agent for treatment of a viral hepatitis infection; c) at least one third pharmaceutical container containing a pharmaceutical at least one second pharmaceutically active agent for treatment of a viral hepatitis infection

In a twenty-third embodiment, the present invention relates to a pharmaceutical composition comprising: a) at least one proteasome inhibitor; b) at least one first pharmaceutically active agent for treatment of a viral hepatitis infection; and c) at least one second pharmaceutically active agent for treatment of a viral hepatitis infection.

In another embodiment, the present invention relates to the use of at least one proteasome inhibitor and at least one pharmaceutically active agent for treatment of a viral hepatitis infection in the manufacture of a medicament for treating human or animal individuals which do not respond or are refractory to treatment with a pharmaceutically active agent for treatment of a viral hepatitis infection, wherein: a) a medicament with said at least one proteasome inhibitor is first administered to an individual which does not respond or is refractory to treatment with a pharmaceutically active agent for treatment of a viral hepatitis infection alone; and b) a medicament with said at least one pharmaceutically active agent is administered subsequent to treatment with the medicament with said at least one proteasome inhibitor.

In another embodiment, the present invention relates to the use of least one proteasome inhibitor and at least a first and at least a second pharmaceutically active agent for treatment of a viral hepatitis infection in the manufacture of a medicament for treating human or animal individuals which do not respond or are refractory to treatment with a pharmaceutically active agent for treatment of a viral hepatitis infection, wherein: a) a medicament with said at least one proteasome inhibitor is first administered to an individual which does not respond or is refractory to treatment with a pharmaceutically active agent for treatment of a viral hepatitis infection alone; b) a medicament with at least one first and at least one second pharmaceutically active agent is administered subsequent to treatment with the medicament with said at least one proteasome inhibitor

In a twenty-fourth embodiment the present invention relates to the use of at least one proteasome inhibitor together with at least one pharmaceutically active agent for treatment of a viral hepatitis infection in the manufacture of a medicament for treating patients which do not respond or are refractory to treatment with a pharmaceutically active agent for treatment of a viral hepatitis infection alone.

In a twenty-fifth embodiment the present invention relates to the use of at least one proteasome inhibitor together with at least one first and at least one second pharmaceutically active agent for treatment of a viral hepatitis infection in the manufacture of a medicament for treating patients which do not respond or are refractory to treatment with a pharmaceutically active agent for treatment of a viral hepatitis infection alone.

In a twenty-sixth embodiment, the invention relates to a method of treating human or animal individuals which do not respond or are refractory to treatment with a pharmaceutically active agent for treatment of a viral hepatitis infection comprising the steps of administering to such individuals first a pharmaceutical composition comprising at least one proteasome inhibitor and subsequent thereto a pharmaceutical composition comprising at least one pharmaceutically active agent for treatment of a viral hepatitis infection.

In a twenty-seventh embodiment, the invention relates to a method of treating human or animal individuals which do not respond or are refractory to treatment with a pharmaceutically active agent for treatment of a viral hepatitis infection comprising the steps of administering to such individuals first a pharmaceutical composition comprising at least one proteasome inhibitor and subsequent thereto a pharmaceutical composition comprising at least one first and one second pharmaceutically active agent for treatment of a viral hepatitis infection.

In a twenty-eighth embodiment, the invention relates to a method of treating human or animal individuals which do not respond or are refractory to treatment with a pharmaceutically active agent for treatment of a viral hepatitis infection comprising the step of administering to such individuals a pharmaceutical composition comprising at least one proteasome inhibitor and at least one pharmaceutically active agent for treatment of a viral hepatitis infection.

In a twenty-ninth embodiment, the invention relates to a method of treating human or animal individuals which do not respond or are refractory to treatment with a pharmaceutically active agent for treatment of a viral hepatitis infection comprising the step of administering to such individuals a pharmaceutical composition comprising at least one proteasome inhibitor and at least one first and at least one second pharmaceutically active agent for treatment of a viral hepatitis infection.

The methods of treatment in accordance with the invention are practiced on human or animal individuals who are in need of such treatment. These may be individuals suffering from a hepatitis viral infection.

In some of above-mentioned embodiments, the viral hepatitis infection is a Hepatitis C Virus (HCV) infection.

### DEFINITIONS

The patent and scientific literature referred to herein establishes knowledge that is available to those of skill in the art. The issued U.S. patents, allowed applications, published foreign applications, and references, including GenBank database sequences, that are cited herein are hereby incorporated by reference to the same extent as if each was specifically and individually indicated to be incorporated by reference.

Terms as set forth hereinafter are generally to be understood in their common sense unless indicated otherwise.

Where an indefinite or definite article is used when referring to a singular noun, e.g., “a”, “an” or “the”, this includes a plural of that noun unless something else is specifically stated.

In the context of the present invention the terms “about” or “approximately” denote an interval of accuracy that the person skilled in the art will understand to still ensure the technical effect of the feature in question. The term typically indicates deviation from the indicated numerical value of ±10%, or of ±5%.

The term “viral hepatitis infection” refers to a disease or condition, such as a hepatitis, caused, induced, or brought about, at least in part, by a viral infection of cells of the liver (e.g., hepatic cells, e.g., without limitation, hepatocytes, hepatic stellate cells or Ito cells, hepatic oval cells, etc.). Such viral infections may be infections with one or more viruses that can specifically infect hepatic cells, such as, for example, without limitation, Hepatitis Virus A, B, C, D, E and G. Alternatively, the hepatitis may be induced by an infection with a virus which may infect and potentially damage the liver as part of a more generalized infection, such as, for example, without limitation, certain Retro-, Herpes, Adeno-, Entero-, Paramyxo-, Toga-, Flavi-, Bunya-, Arena-, Filo-, and Parvoviruses (H. Dancygier, Klinische Hepatologie, Springer-Verlag Berlin Heidelberg 2003, S. 489). More specifically, the hepatitis may be induced by infection with one or more viruses chosen from the group of: Herpes Simplex Virus, HIV, Cytomegalovirus, Epstein-Barr virus, yellow fever virus, mumps virus, rubella virus. The terms “viral hepatitis”, “viral hepatitis infection” and “hepatitis viral infection” are used interchangeably herein. The treatment of a viral hepatitis caused by HCV infection is included in the context of the present invention.

The term “pharmaceutically active agent for treatment of a viral hepatitis infection” refers to a pharmaceutically active compound for which the art recognizes that it can be used for treatment of viral hepatitis infections, such as for the treatment of HCV infections. The term can relate to pharmaceutically active agents as they are authorized by regulatory agencies such as the FDA or the EMEA for treating viral hepatitis infections, in particular HCV infections. The term “pharmaceutically active agent for treatment of a viral hepatitis infection” for the purposes of the present invention does not refer to a proteasome inhibitor or a proteasome-specific inhibitor. Recognition in the art of a pharmaceutically active compound as useful for hepatitis treatment may have occurred at the time the instant invention was made, but nothing herein shall be construed as limiting the instant invention to such compounds. Pharmaceutically active compounds, which are characterized as useful for the therapy of viral hepatitis, and/or gain regulatory approval for such purpose, at a later date, are well within the scope of the instant invention, and are expressly included. A pharmaceutically active agent for treatment of a viral hepatitis infection may be referred to herein for short as “pharmaceutically active agent”, as the context will indicate.

In some embodiments of the kits, methods, composition and pharmaceutical compositions wherein specific pharmaceutically active agents or combinations of pharmaceutically active agents are mentioned, the pharmaceutically active agent(s) is/are the sole pharmaceutically active agent(s) of said kit, method, composition or pharmaceutical composition.

The term “proteasome inhibitor” refers to a compound which can inhibit, reversibly or irreversibly, the proteasome-mediated degradation of ubiquitin-modified peptides and proteins, or proteasome activity.

“Inhibition of proteasome activity” as the term is used herein, shall mean the reduction of proteasome activity inside or outside a cell by at least 5%, at least 10%, at least 15%, 20%, at least 25%, at least 30%, at least 40%, at least 50%, at least 60%, at least 70%, at least 75%, at least 80%, at least 85%, at least 90%, at least 95%, at least 98% or at least 99% compared to the situation where no compound or a compound which is known to not affect proteasome activity is administered. In certain circumstances and cell types, the inhibition of proteasome activity above and beyond a certain level can appear to have untoward effects on the cells. For example, in certain cells, cell viability may drop when the overall residual activity of the proteasome drops below about 40% of the activity seen in these same cells without proteasome inhibitor treatment. In some embodiments of the present invention, that this threshold is generally not surpassed, although it may still be, for example for only a short time, as the activity recovers after removal of the proteasome inhibitor, particularly if the inhibitor is capable of reversibly inhibiting proteasome activity. Generally, proteasome inhibitors which inhibit proteasome activity reversibly, rather than irreversibly, are embodiments of the present invention. In one embodiment, thus, proteasome activity inhibited by an inhibitor of the present invention displays not more than about 30 to 60%, or not more than about 40 to 55%, or not more than about 50% residual activity, such as in cells as are the target of the proteasome inhibitor (e.g., hepatic cells), or in cells acting as a model system, and can provide at maximal inhibition of the proteasome inhibitor, or at the concentration envisaged, for example, as the maximal or steady state concentration in the blood or plasma of a human or animal individual to be treated with the proteasome inhibitor. In one embodiment, the proteasome inhibitor inhibits one or more of the catalytic activities of the 26-S proteasome, or one or more of the postglutamyl-peptide-hydrolyzing (caspase-like, β1-subunit), trypsin-like (β2 subunit), and/or chymotrypsin-like (β5 subunit) catalytic activities. Yet in some embodiments, the proteasome inhibitor inhibits all three, in some embodiments not more than two, and in some embodiments only one of these 26-S proteasome catalytic activities. In some embodiments, the inhibitory activity is observed at nanomolar concentrations in cell culture in vitro, e.g., at concentrations ranging between 1 nM and 1 μM, or 10 nM to 1 μM, or 100 nM to 1 μM, or 1 nM to 100 nM, or 1 nM to 10 μM, or 10 nM to 100 nM.

In another embodiment, the inhibitory activity is observed at concentrations ranging between 1 nM to 10 nM, 10 nM to 10 μM, 100 nM to 10 μM, or 1 μM to 10 μM.

The term “patient” herein is interchangeably used with “a human or animal individual”. A patient herein may be suffering from a viral hepatitis infection, and may therefore be subjected to treatment with the inventive kits, pharmaceutical compositions, combinations and methods as provided herein. In some embodiments, a patient suffers from Hepatitis C virus infection.

### Proteasome Inhibitors

One may use all known proteasome inhibitors; this, however, shall not be interpreted as a limitation to proteasome inhibitors known at the time of the invention, but to include all substances and compositions now or in the future known to inhibit proteasome activity. These include: a) naturally-occurring proteasome inhibitors such as: epoxomycine and eponemycine, aclacinomycine A (also known as aclarubicine), lactacystine and its chemically modified variants, specifically the cell membrane penetrating variant “Clastolactacysteine β-lactone”, b) synthetically prepared proteasome inhibitors such as: modified peptide aldehydes such as N-carbobenzoxy-L-leucinyl-L-leucinyl-1-leucinal (also known as MG132 or zLLL), its boronic acid derivative MG232; N-carbobenzoxy-Leu-Leu-Nva-H (known as MG115); N-acetyl-L-leuzinyl-L-leuzinyl-L-norleuzinal (known as LLnL); N-carbobenzoxy-Ile-Glu(Obut)-Ala-Leu-H (also known as PSI); peptides, such as those that carry at their C-terminal α,β-epoxyketone (also referred to as epoxomicine/epoxomycine or eponemycine), vinyl-sulphones (for example carbobenzoxy-L-leucinyl-L-leucinyl-L-leucin-vinyl sulfone or 4-hydroxy-5-iodo-3-nitrophenylacetyl-L-leucinyl-L-leucinyl-L-leucin-vinyl-sulfone, also referred to as NLVS), glyoxal or boronic acid-residues (for example pyrazyl-CONH(CHPhe)CONH(CHisobutyl)B(OH)₂), also referred to as “PS-341” or Benzoyl(Bz)-Phe-boroLeu, Ph-acetyl-Leu-Leu-boroLeu, Cbz-Phe-boroLeu); pinacol-esters (for example benzyloxycarbonyl(CbZ)-Leu-Leu-boroLeu-pinacol-ester); chemically modified derivatives of naturally occurring proteasome inhibitors such as a β-lactone derivative with the name PS-519 (1R-[1S,4R,5S]]-1-(1-hydroxy-2-methylpropyl)-4-propyl-6-oxa-2-azabicyclo[3.2.0]heptanes-3,7-dione, (molecular formula: C₁₂H₁₉NO₄), being a derivative of the natural proteasome inhibitor lactacystine; dipeptidyl-boronic acid derivatives such as “PS-341” (N-(2,3-pyrazine)carbonyl-L-Phenylalanin-L-leuzin-boronic acid, molecular formula: C₁₉H₂₅BN₄O₄).

Suitable proteasome inhibitors thus include “PS-341” (N-(2,3-pyrazine)carbonyl-L-Phenylalanin-L-leuzin-boronic acid, molecular formula: C₁₉H₂₅BN₄O₄; also commonly known as Bortezomib, and the active ingredient in the pharmaceutical preparation sold under the trade name Velcade®, in use for the treatment of multiple myeloma), “PS-273” (Morpholin-CONH—(CH-napthyl)-CONH—(CH-isobutyl)-B(OH)₂ and its enantiomer “PS-293”, “PS-296” (8-quinolyl-sulfonyl-CONH—(CH-napthyl)-CONH(—CH-isobutyl)-B(OH)₂); “PS-303” (NH₂(CH-Napthyl)-CONH—(CH-isobutyl)-B(OH)₂); “PS-321” (morpholino-CONH—(CH-napthyl)-CONH—(CH-phenylalanine)-B(OH)₂); “PS-334” (CH₃—NH—(CH-napthyl-CONH—(CH-isobutyl)-B(OH)₂); “PS-325” (2-quinole-CONH—(CH-homo-phenylalanin)-CONH—(CH-isobutyl)-B(OH)₂); “PS-352” (Phenyalanine-CH₂—CH₂—CONH—(CH-phenylalanine)-CONH—(CH-isobutyl)1-B(OH)₂); “PS-383” (pyridyl-CONH—(CHpF-phenylalanin2)-CONH—(CH-isobutyl)-B(OH)₂.

Further suitable proteasome inhibitors include (Groll, M., et al, J. Pept. Sci. 2009, 15:58): the aldehydes calpain inhibitor I, Mal-β-Ala-Val-Arg-al and fellutamide B (Hines J., et al., Chem. Biol. 2008, 15:501), syringolin A (Groll M, et al., J. Am. Chem. Soc. 2000, 122:1237) and glidobactin A, members of the new class of Syrbactins; the natural lactones Omuralide, Homobelactosin C and Salinosporamide A (NPI-0052; Salinosporamide A is undergoing clinical Phase 1b trials in drug combination therapy for patients with non-small cell lung cancer, pancreatic cancer, or melanoma), and the vinyl sulfone peptide Ac-Pro-Arg-Leu-Asn-vs; the analogue of epoxomycin carfilzomib, also known as PR-171 (Demo, S. Dak., Cancer Res. 2007, 67:6383); the TMC-95 family of cyclic tripeptides from Apiospora montagnei (Koguchi, Y., J. Antibiot. 2000, 53:105, Kohno, J., Org. Chem. 2000, 65:990), such as TMC-95A and its endocyclic oxindole-phenyl clamp (BIA-1a) and endocyclic biphenyl-ether clamp (BIA-2a) derivatives; the natural cyclic octapeptide argyrin A derived from the myxobacterium Archangium gephyra (Vigneron, N., Science 2004, 304:587), the acetylated tripeptide aldehydes Ac-Leu-Leu-Nle-H and Ac-Arg-Val-Arg-H, the corresponding pegylated tripeptide aldehydes (PEG)₁₉₋₂₅-Leu-Leu-Nle-H and (PEG)₁₉₋₂₅-Arg-Val-Arg-H, as well as their bifunctional equivalents H-Nle-Leu-Leu-(PEG)₁₉₋₂₅-Leu-Leu-Nle-H and H-Arg-Val-Arg-(PEG)₁₉₋₂₅-Arg-Val-Arg-H.

Yet further suitable proteasome inhibitors include (Huang, L., Chen, C H, Current Medicinal Chemistry, 2009, 16:931): CEP1612, a dipeptide aldehyde proteasome inhibitor that is highly selective for the chymotrypsin-like proteolytic activity of the proteasome; ZLVS (ZLLL-vs) and YLVS (YLLL-vs), further examples of vinyl sulfones (herein, -vs is used as shorthand for a vinyl sulfone group); MG-262, a boronate analog of MG132, which exhibits a 100-fold increase in anti-proteasome activity compared to its parent compound; Tyropeptin A, a tripeptide aldehyde natural product isolated from Kitasatospora sp. MK993-dF2, preferentially inhibiting the chymotrypsin-like proteasome activity by binding to the (β5 subunit of the proteasome (Momose, I.; et al., J. Antibiotics 2001, 54:997; Momose, I., et al., Bioorg. Med. Chem. Lett. 2005, 15:1867); Peptide epoxyketones, isolated from various microbials, are small peptides with a ketone epoxide functional group; for example, epoxomycin was derived from Streptomyces hygroscopicus (Hanada, M., et al., J. Antibiot. (Tokyo) 1992, 45:1746), TMC-86 and TMC-89 were isolated from Streptomyces sp. [Koguchi, Y., et al., J. Antibiot. (Tokyo) 2000, 53:63; Koguchi, Y, et al., J. Antibiot. (Tokyo) 2000, 53:967); peptide epoxyketones inhibit the proteasome by covalently modifying the catalytic sites of the β subunits; Carfilzomib (PR-171), an epoxyketone peptide structurally related to epoxomicin, is in Phase 2 clinical trials for patients with relapsed solid tumors including non-small cell lung cancer, small cell lung cancer, ovarian cancer, and renal cancer (Kuhn, D J, et al., Blood 2007, 110:3281); it is also in a phase 2 single-agent trial for patients with multiple myeloma and in a phase 1 study for lymphoma patients; some peptide epoxyketone derivatives, such as dihydroeponemycin analogs, were shown to preferentially target the immunoproteasome (Ho, Y. K., et al., Chem. Biol. 2007, 14:419); PR39 is a naturally occurring antibacterial peptide containing 39 amino acid residues isolated from pig intestine, and was shown to inhibit the proteasome; unlike small tripeptide proteasome inhibitors that bind to the proteolytic active site located at β5 subunit, PR39 binds to the nonproteolytic β7 subunit of the 20S proteasome. PR11 (first 11 residues of PR39 sequence: RRRPRPPYLPR) and its analogs exhibit similar activity to that of PR39; Inhibition of the proteasome by PR11 and PR39 results in accumulation of IκB, a factor that regulates the NF-κB-dependent gene expression pathways; natural products derived from plant sources, such as celastrol, isolated from the traditional herbal medicine “Thunder-god vine”, and withaferin A, isolated from Indian winter cherry, which were shown to inhibit the proteasome at low micromolar concentrations (Celastrol is a triterpene and withaferin A is structurally related to steroids); Gliotoxin, a fungal metabolite structurally related to the epipolythiodioxo-piperazines; green tea polyphenolic catechins such as (−)-epigallocatechin-3-gallate {(−)-EGCG} and its analogs have been widely studied for their possible benefits in cancer prevention; EGCG was reported to potently inhibit the chymotrypsin-like activity of the proteasome in vitro and in cultured tumor cells; Disulfuram, a drug for the treatment of alcohol dependence, was shown to inhibit the proteasome; certain acridine derivatives, a class of anti-cancer agents primarily targeting DNA and topoisomerase II, also having proteasome inhibiting activity, e.g., tetra-acridine; certain derivatives of betulinic acid, e.g., 3′,3′-dimethylsuccinyl betulinic acid; in contrast to BA a proteasome activator, many BA derivatives inhibit the proteasome; similarly, certain derivatives of glycyrrhetinic acid (GLA) may be potent inhibitors of the proteasome, and such inhibitors are envisaged by the present invention.

Yet further suitable proteasome inhibitors include: NEOSH-101, also known as OSH-101, a tetrapeptide aldehyde in clinical trials for androgenetic alopecia; CEP-18770, a P2 threonine boronic acid derivative under development for, e.g., multiple myeloma; IPSI001, IPSI007, as well as MLN2238 and its prodrug MLN9708, under development for indications in oncology by Millennium Pharmaceuticals/Takeda; ONX 0912 (formerly PR-047 (1) by Proteolix, Inc.; Peese, K., Drug Discovery Today 2009, 14: 905), a proteasome inhibitor based on the same novel chemistry as carfilzomib, and ONX 0914 (formerly PR-957; Muchamuel, T., et al., Nature Medicine 2009, 15:7) an inhibitor of the immunoproteasome, both being developed by Onyx Pharmaceuticals; AA-102, an anticancer agent being developed by Bionovo, Inc., 26 S PI, a proteasome inhibitor being developed by Ergon Pharmaceuticals for oncology and other indications; AVR-147, a development candidate by Advanced Viral Research, Corp., in oncology; BU-32 (pyrazyl-2,5-bis-CONH(CHPhe)CONH(CHisobutyl)-B(OH)₂, NSC D750499-S; Aygin, J K, et al-., Breast Cancer Res. 2009, 11:R74), 4E12, a non-peptidyl small molecule proteasome inhibitor identified by Telik, Inc., and intended for development in oncology; and Compound 13 and Compound 20 (Purandare, A S, et al., Am. Assoc. Cancer Res Annual Meeting 2007, 98^{th}: April 15, Abs. 717), two lactam boronic acid proteasome inhibitors having high activity (low nM IC₅₀ values) as well as high specificity (>100 fold selective against chymotrypsin, trypsin, elastase and Factors Xa, Xia and VIIa).

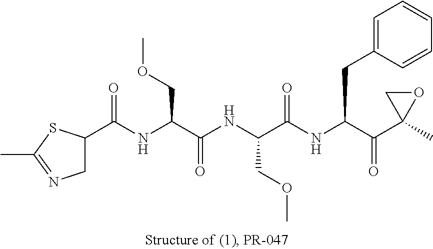

Yet further suitable proteasome inhibitors include: ALLnL, ALLnM; LLnV; DFLB (dansyl-Phe-Leu-boronate); Ada-(Ahx)₃-(Leu)₃-vs; YU101 (Ac-hFLFL-ex), MLN519, and the semi-carbazone S-2209 (Baumann et al., Brit. J. Haematology 144: 875-886, 2009) as well as its structural analogs Compound 1 to Compound 6 and Compound 8 as given in Leban, J., et al., Bioorg. Med. Chem. 2008, 16:4579; S-2209 (S,S,S-[1-[1-[1-Benzyl-2-(2,4-dioxo-imidazolidin-1-ylimino)-ethylcarbamoyl]-2-(1H-indol-3-yl)-ethylcarbamoyl]-2-(1H-indol-3-yl)-ethyl]-carbamic acid benzyl ester) is an embodiment of a proteasome inhibitor.

Some proteasome inhibitors act on one or more of the postglutamyl-peptide-hydrolyzing (caspase-like, β1-subunit), trypsin-like (β2 subunit), and/or chymotrypsin-like (β5 subunit) activities found within the 26S proteasome (Groll, M., et al., J. Pept. Sci. 2009; 15:58; Löwe J, et al., Science 1995, 268:533; Groll M, et al., Nature 1997; 386:463). Proteasome inhibitors may thus include di-, tri,- tetra-, penta-, hexa-, hepta-, octa-, nona-peptide aldehydes or peptide aldehydes having ten or more amino acids such as 15, 20, 30, 40 or more amino acids.

Such di-, tri,- tetra-, penta-, hexa-, hepta-, octa-, nona-peptide aldehydes or peptide aldehydes having ten or more amino acids may carry at their C-terminus an α,β-epoxyketone functionality, a vinyl-sulphone functionality, a glyoxal functionality, a boronic acid functionality, pinacol ester functionality or other functionalities.

Such di-, tri,- tetra-, penta-, hexa-, hepta-, octa-, nona-peptide aldehydes or peptide aldehydes having ten or more amino acids may comprise natural or non-natural amino acids. Such di-, tri,- tetra-, penta-, hexa-, hepta-, octa-, nona-peptide aldehydes or peptide aldehydes having ten or more amino acids may also be chemically modified by hydrogenation, dehydrogenation, hydroxylation, dehydroxylation, acylation, deacylation, alkylation, dealkylation, pegylation, hesylation, glycosylation and the like.

Such di-, tri-, tetra-, penta-, hexa-, hepta-, octa-, nona-peptide aldehydes or peptide aldehydes having ten or more amino acids and which optionally may carry α,β-epoxyketone functionality, a vinyl-sulphone functionality, a glyoxal functionality, a boronic acid functionality, pinacol ester functionality or other functionalities at their C-terminus may also comprise natural or non-natural amino acids.

Such di-, tri,- tetra-, penta-, hexa-, hepta-, octa-, nona-peptide aldehydes or peptide aldehydes having ten or more amino acids and which optionally may carry α,β-epoxyketone functionality, a vinyl-sulphone functionality, a glyoxal functionality, a boronic acid functionality, pinacol ester functionality or other functionalities at their C-terminus may also be further chemically modified by hydrogenation, dehydrogenation, hydroxylation, dehydroxylation, acylation, deacylation, alkylation, dealkylation, pegylation, hesylation, glycosylation and the like.

Such di-, tri,- tetra-, penta-, hexa-, hepta-, octa-, nona-peptide aldehydes or peptide aldehydes having ten or more amino acids and which optionally may carry α,β-epoxyketone functionality, a vinyl-sulphone functionality, a glyoxal functionality, a boronic acid functionality, pinacol ester functionality or other functionalities at their C-terminus and may also be further chemically modified by hydrogenation, dehydrogenation, hydroxylation, dehydroxylation, acylation, deacylation, halogenations, alkylation, dealkylation, pegylation, hesylation, glycosylation and the like, may also comprise natural or non-natural amino acids.

In some embodiments, a proteasome inhibitor of the invention inhibits one or more of the above catalytic activities of the 26-S proteasome, or one or more of the postglutamyl-peptide-hydrolyzing (caspase-like, β1-subunit), trypsin-like (β2 subunit), and/or chymotrypsin-like (β5 subunit) catalytic activities.

In some embodiments, the proteasome inhibitor inhibits all three, or not more than two, or only one of these 26-S proteasome catalytic activities.

In some embodiments, the inhibitory activity is observed at nanomolar concentrations in cell culture in vitro, e.g., at concentrations ranging between 1 nM and 1 μM, or 10 nM to 1 μM, or 100 nM to 1 μM, or 1 nM to 100 nM, or 1 nM to 10 μM, or 10 nM to 100 nM.

In another embodiment, the inhibitory activity is observed at concentrations ranging between 1 nM to 10 nM, 10 nM to 10 μM, 100 nM to 10 μM, or 1 μM to 10 μM.

As these proteolytic activities can also be found in cellular proteases, proteasome inhibitors may not only inhibit the proteasome as described above, but also further cellular proteases.

Proteasome inhibitors can act by inhibiting the respective proteasomal activity irreversibly, e.g., by binding covalently to an active site within the proteasome, or reversibly. Proteasome inhibitors acting in a reversible manner are suitable for the pharmaceutical compositions, the kits, the uses; the pharmaceutical compositions and the methods as described herein.

“Specific proteasome inhibitor”, or “specific inhibition” of an activity of the proteasome by an inhibitor, herein shall mean that the inhibitor reduces the said activity by 50% at a concentration (termed IC₅₀) that is lower by at least a factor of 1/2, 1/3, 1/5, 1/10, 1/20, 1/50, 10⁻², 5×10⁻³, 2×10⁻³, 10⁻³, or less, than the IC₅₀ of that same inhibitor for the inhibition of another, or many other, or, in some embodiments, any other relevant activity in question, e.g., a proteolytic activity not associated with the proteasome.

For example, a specific proteasome inhibitor can be at least twice as potent with respect to the inhibition of any one, or more, of the 26S proteasome catalytic activities than with respect to the inhibition of, e.g., a lysosomal protease or an HIV protease, or at least three times as potent, or at least five times as potent, etc. Such specific proteasome inhibitors include, without limitation, the proteasome inhibitors PS-519, PS-341 (Bortezomib), PS-273, and S-2209. These proteasome inhibitors are potent, specific for the proteasome and substantially do not block other cellular proteases. The proteasome inhibitors PS-341 and PS-519 have moreover been tested pre-clinically in animal models and in humans for clinical studies (cancer patients).

The inhibitory activity on the proteasome of a candidate proteasome inhibitor may be assessed by, for example, the assay described in Adams, J., et al., Cancer Research 1999, 59:2615, or the commercially available Proteasome Glo™ Assay (Promega Corp. Madison Wis., USA). These assays may be used to determine an IC₅₀ for the candidate proteasome inhibitor respective the one or more proteasome catalytic activities; any other suitable assay may then be used to determine an IC₅₀ for the candidate proteasome inhibitor respective another relevant activity in question; and these IC₅₀ values may hence be compared to obtain a measure of the specificity of the inhibitory activity of the candidate proteasome inhibitor.

### Pharmaceutically Active Agents for Treatment of a Viral Hepatitis

Pharmaceutically active agents for treatment of a viral hepatitis infection can be broadly classified in two categories, or groups.

Pharmaceutically active agents for treatment of a viral hepatitis infection of the first category support or assist the body's natural response in dealing with a viral infection. Pharmaceutically active agents for treatment of a viral hepatitis of the second category will interfere with the function of a viral target, such as a virus-specific protease or polymerase.

For the kits, pharmaceutical compositions, combinations and methods of the instant invention, a proteasome inhibitor is combined with at least one pharmaceutically active agent for treatment of a viral hepatitis infection, and optionally with at least two pharmaceutically active agents for treatment of a viral hepatitis infection. Where the proteasome inhibitor is combined with only one pharmaceutically active agent for treatment of a viral hepatitis infection, this pharmaceutically active agent for treatment of a viral hepatitis infection may be freely selected from either of the above described categories.

However, where the proteasome inhibitor is combined with more than one pharmaceutically active agent for treatment of a viral hepatitis infection, it is one embodiment of the instant invention, that at least one of the more than one pharmaceutically active agents for treatment of a viral hepatitis infection is selected from either of the above described categories. Hence, where a first pharmaceutically active agent for treatment of a viral hepatitis infection is selected from a first category of pharmaceutically active agents for treatment of a viral hepatitis infection, an optional second can be selected from another category of pharmaceutically active agents for treatment of a viral hepatitis infection. E.g., where the first pharmaceutically active agent for treatment of a viral hepatitis infection is selected from the group of pharmaceutically active agents for treatment of a viral hepatitis infection which support or assist the body's natural response in dealing with viral infections, an optional second can be chosen from the group of pharmaceutically active agents for treatment of a viral hepatitis infection which interfere with the function of a viral target, such as a virus-specific protease or polymerase, or vice versa.

Pharmaceutically Active Agents for Treatment of a Viral Hepatitis Infection which Assist the Body's Natural Response to Viral Infection

In principle such pharmaceutically active agents which will be administered in addition to proteasome inhibitors, and can, in addition to specific proteasome inhibitors, include, without intent to limitation, cytokines, such as interferons and their derivatives, and interleukins such as IL-1, IL-2, IL-6, IL-7, IL-8, IL-12, IL-15, IL-18, IL-21, IL-22, IL-28, and IL-29, inhibitors of viral enzymes, e.g., protease inhibitors e.g., telaprevir, boceprevir, ITMN-191, SCH 900518, TMC435, BI201335 and MK-7009, nucleoside analogs, nucleotide analogs and non-nucleoside analogous inhibitors of viral enzymes which may, for example, inhibit a viral polymerase and/or a viral protease, steroids, thymosin alpha 1, vaccines including vaccines allowing for passive and active vaccination, therapeutic and prophylactic vaccination, glycyrrhizin, immunomodulators, e.g., Thymosin, ME3738, SCV-07, Alinia, Oglufanide, IPH-1101, CYT 107, or EGS-21, and/or immunosuppressants and/or inhibitors of assisted protein folding, e.g., ciclosporin and derivatives thereof, e.g., SCY-635, DEBIO-025, NIM811, Silibinin, Nitazoxanide, A-831, KPE02001003, TCM700C, PYN-17, BIT225, JTK-652, BMS-791325BMS-791325, ACH-806 (GS-9132), amantadine or rimantadine and derivatives thereof, and azathoprine.

Pharmaceutically active agents which support or assist the body's natural response in dealing with viral infections may comprise interferons, interleukins, steroids, immunomodulators, immunosuppressants and inhibitors of assisted protein folding.

Interferons can be used as pharmaceutically active agents which support or assist the body's natural response in dealing with a viral infection.

The term “interferon” refers to the various forms of interferons including their derivatives. Thus the term includes interferon alpha-1, alpha-2, beta, gamma, delta, lambda and omega as well as the glycosylated, pegylated and hesylated forms thereof, and other forms wherein the interferon is fused or otherwise conjugated to another moiety conveying desirable properties to the overall molecule, e.g., albinterferon (Albuferon, alb-IFN, Zalbin), a fusion polypeptide of interferon with albumin. The term further includes: PEG-IFN alpha-2a, alpha-2b or lambda, Locteron, Omega IFN, Medusa IFN, DA-3021, EMZ702, Infradure, IL-28, IL-29, Veldona (Oral Interferon alfa, Amarillo Biosciences, Amarillo, Tex., USA), Soluferon and Belerofon (see e.g., Thompson, A., et al., J. Hepatology, 50:184 (2009)).

For example, Albinterferon is a genetic fusion polypeptide of albumin and interferon alpha-2b with a longer half life than pegylated interferons. A phase 2 study comparing different doses of albinterferon alpha-2b and ribavirin with PEG-interferon alpha-2a and ribavirin indicated similar sustained virologic response rates with a better tolerability of albinterferon alpha-2b based treatment. Based on the encouraging findings from the phase 2 study, the efficacy and safety of albinterferon alpha-2b administered every two weeks in combination with ribavirin for 48 weeks and 24 weeks in patients infected with HCV genotype 1 and 2/3, respectively, was investigated in two phase 3, randomized, active controlled, multi-center studies. Both studies ACHIEVE-1 and ACHIEVE-2 were designed to demonstrate non-inferiority of the albinterferon alpha-2b regimes compared with PEG-interferon alpha-2a. Both studies achieved the primary objective.

Locteron is a controlled-release interferon alpha-2b which is injected every 2 weeks. In a short term study controlled release interferon alpha-2b showed less flu like symptoms than PEG-interferon alpha-2b injected every week indicating that the controlled-release formulation may have a better tolerability.

PEG-interferon lambda is a pegylated type III interferon that binds to a unique receptor with more limited distribution than the type I interferon receptor. In a phase 1b study the mean decline of HCV-RNA in patients with relapsed HCV genotype 1 infection was 1.9-3.6 log 10 IU/mL after 4 weeks of re-treatment with PEG-interferon lambda. PEG-interferon lambda is currently investigated in combination with ribavirin.

Further interferon varieties under development for hepatitis treatment include Omega IFN, Medusa IFN, DA-3021, EMZ702, Infradure, IL-28, IL-29, Veldona, and Belerofon. Also envisaged is Soluferon.

In some embodiments described herein, an interferon is a pegylated interferon alpha (PEG-interferon-alpha), for example PEG-interferon-alpha 2a or 2b.

Pharmaceutically Active Agents for Treatment of a Viral Hepatitis which Interfere with the Function of a Viral Target

Pharmaceutically active agents for treatment of a viral hepatitis of the second category, which interfere with the function of a viral target, may be agents that inhibit viral enzymes such as a virus-specific protease or polymerase or a viral envelope protein. In case of HCV infections this may be inhibitors of, e.g., the HCV NS3/4A protease and/or the HCV NS5B polymerase.

Nucleoside analogs, nucleotide analogs and non-nucleoside analog inhibitors of viral enzymes include lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 and ACH-806 (GS-9132). An example of a nucleoside analog is ribavirin. Another example of a nucleoside analog is viramidine.

For example, the structure identification of the NS3/4A protease and the HCV NS5B polymerase and the development of a (sub) genomic replicon system have enabled the development and testing of HCV specific compounds. Further attractive targets within the HCV genome for antiviral therapy are the envelope proteins which are involved in HCV entry and NS5A which is involved in replication and in interferon alpha resistance. The clinical development of NS3/4A protease inhibitors is currently most advanced.

### HCV Protease Inhibitors

The NS3/4A protease has key functions in the hepatitis C virus replication cycle. NS3/NS4A cleaves at four downstream sites in the polyprotein to generate the N-termini of the NS4A, NS4B, NS5A, and NS5B proteins. The NS3/4A serine protease has also been shown to cleave and inactivate the host proteins Trif and Cardif. Both proteins have important roles in the interferon response mediated by TLR3 and RIG-I, respectively.

Furthermore, it has been shown that NS3 is not only a protease but also an integral part of the viral RNA replication complex, functions as a RNA helicase and a nucleotide triphosphatase (NTPase). Due to the multiple functions, NS3 is an attractive target for anti-HCV therapy. Several protease inhibitors were investigated in clinical trials. Monotherapy with protease inhibitors ciluprevir, telaprevir and boceprevir was shown to be effective in lowering the viral load. Clinical evaluation of telaprevir and boceprevir is most advanced. Both protease inhibitors showed a rapid occurrence of drug resistant HCV strains within 2 weeks of therapy indicating that protease monotherapy is not sufficient for treatment of patients with chronic hepatitis C.

The peptidomimetic inhibitor of the NS3/4A serine protease telaprevir showed a 3 log 10 IU/mL decline of HCV RNA during the first 2 days of monotherapy in patients infected with HCV genotype 1 and previous non response to interferon based antiviral treatment. Combination therapy of telaprevir with PEG-interferon alpha-2a and ribavirin was effective in preventing the rapid occurrence of resistance. The combination therapy of PEG-interferon alpha-2a/ribavirin/telaprevir was investigated in the PROVE1 and 2 studies. Both studies are completed and telaprevir is one of the first STAT-C compound for which sustained virologic response rates have been reported for the combination therapy with PEG-interferon alpha-2a and ribavirin. In both trials triple therapy was given for 12 weeks. The sustained virologic response rates in PROVE1 and PROVE2 were 67% and 69% in patients treated with PEG-interferon alpha-2a/ribavirin/telaprevir for 12 weeks followed by PEG-interferon/ribavirin for 36 or 12 weeks, respectively. The sustained virologic response rates in these telaprevir arms were significantly higher compared with the sustained virologic response rates in the standard of care control arms (41% and 46% in PROVE1 and PROVE2 respectively). Overall, the PROVE-studies confirm that protease inhibitors are able to increase sustained virologic response rates in patients with HCV genotype 1 infection. Furthermore, the PROVE2 study indicates that by addition of telaprevir to standard therapy higher sustained virologic response rates can be achieved with shorter treatment duration. The results of the PROVE2-trial provide evidence that ribavirin has additive antiviral activity to telaprevir and PEG-interferon alpha-2a.

Boceprevir, another NS3/4A serine protease inhibitor, binds reversibly to the NS3 protease active site and has potent activity in the Replicon system alone and in combination with interferon alpha-2b. Recently, the final results of the HCV SPRINT-1 study assessing safety and efficacy of boceprevir in combination with PEG-interferon alpha-2b (1.5 μg/kg/week) and ribavirin in treatment naïve patients with chronic hepatitis C genotype 1 infection were presented. The triple combination arms with a total treatment duration of 48 weeks with or without a 4 weeks PEG-interferon-alpha2b/ribavirin lead-in were associated with significantly higher sustained virologic response rates than the low dose ribavirin arm and the standard of care control arm (75% and 67% vs 36% and 38%, respectively).

ITNM-191, SCH 900518, TMC435, BI201335 and MK-7009 are novel NS3/4A protease inhibitors currently in clinical trials. ITMN-191 is a potent HCV NS3/4A protease inhibitor that achieves high liver concentrations following oral administration. ITNM-191 in combination with PEG-interferon alpha-2a/ribavirin showed a stronger decline of HCV RNA compared with PEG-interferon alpha-2a/ribavirin standard of care after two weeks of treatment (4.7-5.7 log 10 IU/mL vs 2.0 log 10 IU/mL). After 2 weeks, 13-57% of patients in the triple therapy arm while no patient in the standard of care arm showed undetectable HCV RNA. SCH-900518 with and without ritonavir boostering showed robust reductions in HCV RNA levels in both treatment-experienced and naïve HCV genotype 1-infected patients (4.01 log 10 IU/mL and 4.5 log 10 IU/mL vs 0.09-0.19 log 10 IU/mL after 8 days in patients treated with SCH 900518 400 mg twice/day plus PEG-interferon alpha-2a/ribavirin plus ritonavir 100 mg/d and SCH 900518 800 mg thrice/day plus PEG-interferon alpha-2a/ribavirin, respectively, vs. patients receiving PEG-interferon alpha-2a/ribavirin alone). TMC435 administered for 4 weeks in combination with PEG-interferon-alpha2a/ribavirin was well tolerated and demonstrated potent antiviral activity in HCV genotype 1 infected treatment-experienced patients (4.3-5.3 log 10 IU/mL in the TMC435 arms vs 1.5 log 10 IU/mL in the control arms). BI 201335 was investigated as monotherapy for 14 days and in combination with PEG-interferon alpha-2a/ribavirin for 28 days in experienced patients and showed a median HCV RNA decline of 3-4.2 log 10 IU/mL in monotherapy and 4.8-5.3 log 10 IU/ml in combination therapy. MK-7009 is a noncovalent competitive inhibitor of HCV NS3/4A protease. In treatment naïve patients MK-7009 was administered for 28 days in combination with pegylated interferon-alpha/ribavirin. The rapid virologic rate was higher in patients treated with triple therapy than in patients treated with standard of care (68.8-82.4% vs 5.6%). All new compounds were relatively safe and well tolerated in monotherapy as well in combination with standard of care and will be further developed for HCV treatment (Kronenberger, B., Zeuzem, S., Annals of Hepatology 2009; 8: 103).

Further protease inhibitors under development for hepatitis treatment include BMS790052, VBY-376, and TMC-435350 (Thompson, A., et al., J. Hepatology, 50:184 (2009)); RG7227 (ITMN-191), BI201335, narlaprevir (SCH900518), VX-813, VX-985, ABT-450, ACH-1095, ACH-1625, AVL-181, BILN-2061, GS-9256, IDX-320, ITMN-5489, and PHX1766.

### HCV Polymerase Inhibitors

Two classes of NS5B polymerase inhibitors, nucleoside and non-nucleoside polymerase inhibitors, have been developed. Nucleoside analogue polymerase inhibitors are converted into triphosphates by cellular kinases and incorporated into the elongating RNA strand as chain terminators. Generally, they show similar efficacy against all HCV genotypes. The mechanisms of action of non-nucleoside polymerase inhibitors are different from that of nucleoside polymerase inhibitors. Therefore, cross resistance between these two classes is unlikely to occur. Several structurally distinct non-nucleoside inhibitors of the HCV RNA-dependent RNA-polymerase NS5B have been reported to date, including benzimidazole, benzothiadiazine, and disubstituted phenylalanine/thiophene or dihydropyranone derivatives. They target different sites within the polymerase. Different resistance profiles due to distinct target sites can be expected for the class of non-nucleoside inhibitors. As with protease inhibitors a single mutation may already confer resistance to non-nucleoside polymerase inhibitors. In contrast to nucleoside polymerase inhibitors, a restricted spectrum of activity of non-nucleoside polymerase inhibitors against different HCV genotypes and subtypes has been described.

### Nucleoside Analogs

Valopicitabine was the first nucleoside analogue polymerase inhibitor tested in patients with chronic hepatitis C. Valopicitabine showed antiviral activity in monotherapy (mean HCV-RNA decline 0.15-1.21 log 10 IU/mL after 14 days in patients infected with HCV genotype 1 and prior non response to interferon based antiviral treatment) and in combination therapy with interferon alpha (mean HCV-RNA decline 3.75-4.41 log 10 IU/mL after 36 weeks in treatment naïve patients infected with HCV genotype 1). The nucleoside analogue R1479 (4′-azidocytidine) is a potent inhibitor of NS5B-dependent RNA synthesis and hepatitis C virus replication in cell culture. R1626 (Balapiravir) is a prodrug of R1479 (4′-azidocytidine). R1626 was investigated in treatment naïve patients with HCV genotype 1 infection in combination with PEG-interferon alpha-2a and ribavirin. After 48 weeks (4 weeks R1626 plus PEG-interferon alpha-2a with or without ribavirin followed by 44 weeks of PEG-interferon alpha-2a plus ribavirin) the virologic response rates were 52-84% in the R1626 treatment arms and 65% in the control arm with PEG-interferon alpha-2a/ribavirin. Remarkably, end of treatment response was higher in the ribavirin arm than in the non-ribavirin arm (84% vs 52-66%) indicating that ribavirin has additional effects on treatment antiviral activity to polymerase inhibitors.

R1626, a prodrug of a cytidine analog, is a nucleoside inhibitor currently in phase 2 development. When used in combination with PEG-IFN and RbV for 4 weeks, the mean maximal viral load reduction from baseline was 5.2 log 10 IU/mL. R7128 is another nucleoside analogue NS5B polymerase inhibitor. Non responders treated with R7128 1,500 mg twice daily showed a mean viral decline of 2.7 log 10 IU/mL after 14 days of therapy. R7128 is currently evaluated in combination with PEG-interferon alpha-2a and ribavirin in treatment naïve patients with chronic HCV genotype 1 infection. The week 4 rapid virologic response rates in patients treated with PEG-interferon alpha-2a, ribavirin plus R7128 500 mg or 1,500 mg twice daily were 30% and 85%, respectively, and 10% in patients treated with PEG-interferon alpha-2a and ribavirin without R7128. R7128 also showed antiviral activity against HCV genotype 2/3 in vitro.

### Nucleotide Analogs

IDX184 is a liver-targeted nucleotide prodrug designed to enhance formation of its active triphosphate in the liver while minimizing systemic exposure of the parent drug and its nucleoside metabolite. Oral administration of IDX184 to HCV-infected chimpanzees resulted in potent antiviral activity (mean HCV-RNA decline after 4 days 1.4 to 3.8 log 10 copies/mL). The antiviral activity was achieved with low systemic levels of the parent drug and its nucleoside metabolite.

### Non-Nucleoside-Analogous Inhibitors of Viral Enzymes

HCV-796 is a non-nucleoside polymerase inhibitor that has demonstrated potent antiviral activity in vitro and in patients with chronic hepatitis C. Monotherapy showed a maximum antiviral effect after 4 days of treatment with a mean HCV RNA reduction of 1.4 log 10 IU/mL. The combination of HCV-796 and PEG-interferon alpha-2b produced a mean viral reduction of 3.3-3.5 log 10 IU/mL after 14 days of treatment compared to 1.6 log 10 IU/mL with PEG-interferon alpha-2b alone.

Filibuvir (PF 00868554) showed in monotherapy of patients with chronic HCV genotype 1 infection a dose-dependent inhibition of viral replication, with maximum reductions in HCV RNA ranging from 0.97 to 2.13 log 10 IU/mL. In treatment naïve patients with HCV genotype 1 infection triple therapy with PEG-interferon alpha-2a and ribavirin was associated with a rapid virologic response rate of 60-75% while no patient in the placebo arm achieved a rapid virologic response.

The non-nucleoside polymerase inhibitors VCH-916, ANA598, BI 207127 and VCH-222 were investigated only in monotherapy so far. VCH-916 showed a maximum HCV-RNA decline ranging between 0.2 and 2.5 log 10 IU/mL within 14 days of treatment. ANA598 showed a decline of HCV RNA after 3 days of monotherapy ranging between 0.4 and 3.4 log 10 IU/mL. 43 ANA598 was combined in vitro with interferon alpha, the HCV NS3/4 protease inhibitor telaprevir, the NS5B nucleoside polymerase inhibitor PSI-6130, and the TLR7 agonist ANA773, respectively. The in vitro combination studies demonstrated additive to synergistic antiviral effects of ANA598 in combination with other anti-HCV agents having distinct mechanisms of action and non-overlapping resistance profiles. The study indicates that combination therapy may produce a greater viral load reduction and potentially delay the emergence of drug resistance in vivo. BI 207127 monotherapy showed an HCV-RNA decline after 5 days ranging between 0.6 and 3.1 log 10 IU/mL in patients with chronic hepatitis C genotype 1 infection. Similar to ANA598, no increase in HCV RNA levels was observed during short term BI 207127 monotherapy. For VCH-222 only preliminary efficacy results on the first 4 treatment-naïve patients with chronic HCV genotype 1 infection treated for 3 days are available showing a decline of HCV-RNA ranging between 3.2 and 4.2 log 10 IU/mL. MK-3281, ABT-072 and ABT-333 are additional nonnucleoside polymerase inhibitors in development.

Further polymerase inhibitors under development for hepatitis treatment include R1728, VCH-759, GS9190, BMS-650032, BE-868554, MK-3281 (Thompson, A., et al., J. Hepatology, 50:184 (2009)); PSI-7977, PSI-7851, PSI-938, BILB 1941, IDX-375, PSI-7851, PSI-7977 (single isomer of PSI-7851).

### HCV-Entry Inhibitors

Chronic hepatitis C is characterized by a high turnover of infected cells and continuous de novo infection of target cells. Due to the vital role of de novo infection in maintenance of HCV infection, blocking of de novo infection is a potential target for antiviral therapy. A target to block de novo infection is the HCV envelope protein E2. A fully humanized monoclonal antibody to a linear epitope of HCV E2 glycoprotein MBL-HCV1 that neutralizes pseudoviruses from multiple HCV genotypes was developed. The antibody was shown to completely neutralize infectious HCV particles in cell culture. Three chimpanzees received a single dose of the Anti-E2 antibody intravenously before challenge with HCV 1a strain H77. No HCV RNA was detected in the serum of 250 mg/kg dosed chimpanzees through week 20 while the 0 mg/kg and 50 mg/kg dosed chimpanzees both became infected by day 14. These findings indicate that a human monoclonal antibody directed to a conserved epitope of the HCV E2 glycoprotein has the potential to neutralize infectious, replication competent HCV and may prevent infection. The blocking of viral entry into a target cell by an agent may herein be referred to as “entry inhibition” or “envelope protein inhibition”, regardless of the mechanism of the blocking action.

Combinations and Other Approaches of Interfering with a Viral Target

One may also consider using combinations of inhibitors of viral targets in the context of the present invention. Thus one may use e.g., an inhibitor of an HCV protease together with inhibitors of HCV polymerase and/or an HCV envelope protein. Of course these agents may be additionally combined with e.g., active agents which support or assist the body's natural response in dealing with viral infections such as interferons, interleukins, steroids, immunomodulators, immunosuppressants and inhibitors of assisted protein folding.

The nucleoside polymerase inhibitor R7128 and the protease inhibitor ITNM-191 showed substantial antiviral activity in patients with chronic hepatitis C. The INFORM-1 trial is the first trial to investigate the combination of a nucleoside polymerase inhibitor and a protease inhibitor in patients with chronic hepatitis C. Both compounds have different resistance profiles and thus are good candidates for combination therapy. After 14 days of combination therapy (with yet lower doses for both compounds), a decline of HCV-RNA ranging between 2.9 and 5.0 to log 10 IU/mL was observed. One patient had undetectable HCV-RNA. No viral rebound was observed.

Other compounds which may be used as pharmaceutically active agents aside from proteasome inhibitors may act both on viral targets and host cell factors.

For example, cyclophilins are ubiquitous proteins in human cells that are involved in protein folding. Moreover, cyclophilins participate in HCV replication. It was shown that cyclophilin B binds to the HCV NS5B polymerase and stimulates its RNA-binding activity. Cyclophilin inhibitors show strong antiviral activity in vitro and in vivo. The cyclophilin inhibitor DEBIO-025 showed dual antiviral activity against HCV and HIV in a phase 1 trial with HCV/HIV co-infected patients. DEBIO-025 was investigated in combination with PEG-interferon alpha-2a and ribavirin in HCV genotype 1 null responders to previous PEG-interferon/ribavirin combination therapy. Triple combination therapy showed a HCV RNA decline after 29 days of 0.88-2.38 log 10 IU/mL in the different dosing arms while no antiviral activity was observed in patients receiving DEBIO-025 monotherapy.

NIM811 is another oral non-immunosuppressive cyclophilin inhibitor which has in vitro activity against HCV. In patients with HCV genotype 1 infection with previous relapse to PEG-interferon/ribavirin therapy, NIM811 in combination with PEG-interferon alpha-2a showed a mean HCV RNA decline of 2.78 log 10 IU/mL after 14 days compared with a 0.58 log 10 decline of HCV RNA in the PEG-interferon alpha-2a monotherapy arm.

SCY-635 is also a non-immunosuppressive analog of cyclosporine A that exhibits potent suppression of HCV RNA replication in vitro. SCY 635 binds to human cyclophilin A at nanomolar concentrations. Different doses of SCY-635 were investigated in patients infected with HCV genotype 1 and viral load above 100,000 IU/mL. Consistent decreases in plasma HCV RNA were observed in the highest dose group (mean nadir values were 2.20 log 10 IU/mL).

Oral silibinin is widely used for treatment of hepatitis C, but its efficacy is unclear. Intravenous silibinin was investigated in non-responders to prior interferon-based antiviral therapy and showed a significant decline in HCV RNA between 0.55 to 3.02 log 10 IU/mL after 7 days and a further decrease after additional 7 days in combination with PEG-interferon alpha-2a and ribavirin in the range between 1.63 and 4.85 log 10 IU/mL. Next, intravenous silibinin was investigated as rescue treatment for patients with chronic hepatitis C who were still HCV-RNA positive after 24 weeks of treatment with PEG-interferon alpha-2a/ribavirin. After 24 weeks of treatment with standard of care the patients received additionally 20 mg/kg/d silibinin intravenously for 15 days. Thereafter PEG-interferon/ribavirin was continued. After 15 days of intravenous silibinin therapy HCV-RNA decreased in all patients and 7 out of 9 patients achieved undetectable HCV RNA plasma levels. After the end of silibinin administration patients were followed for at least 12 weeks. In one patient HCV-RNA increased to 100 IU/mL, and a second course of intravenous silibinin for 15 days was given. HCV-RNA became negative again and remained negative so far.

Nitazoxanide is a thiazolide anti-infective with activity against a number of protozoa, bacteria, and viruses. It is FDA approved for treatment of cryptosporidium and giardia. Nitazoxanide inhibits replication of hepatitis C virus, hepatitis B virus, and rotavirus in vitro. Based on its broad antiviral activity, the mechanism of action is likely through cellular processes rather than specific anti-viral targets. Rossignol et al. recently reported that the use of nitazoxanide in combination with PEG-interferon alpha-2a with or without ribavirin among treatment-naive hepatitis C patients infected with genotype 4 significantly improved viral response rates compared to the standard of care (PEG-interferon alpha-2a plus ribavirin). The sustained virologic response rates were 79% and 64% in patients treated with PEG-interferon alpha-2a/ribavirin/nitazoxanide and PEG-interferon alpha-2a/nitazoxanide, respectively, versus 45% in patients treated with PEG-interferon alpha-2a/ribavirin.

NS4B and NS5A are further nonstructural proteins that result from the processing of the large HCV protoprotein by the NS3/4A protease. Although the exact functions of these proteins are just being elucidated, it has been postulated that they could serve as new targets for antiviral intervention (Sklan, E. N, and Glenn, J. S.; Hepatitis C Viruses: Genomes and Molecular Biology, Tan, S. L. (Ed.), Horizon Bioscience 2006, Norfolk (UK); Macdonald, A., Harris, M., J. Gen. Virol. 2004, 85:2485-2502). Compounds putatively acting via inhibition of NS5A include BMS-790052 (Bristol-Myers Squibb), IDX-NS5A, (Idenix), PPI-461 and PPI-1301 (Presidio), AZD2836 and AZD7295 (Arrow Therapeutics, acquired by AstraZeneca), and EDP-239 (Enanta). These and other substances, which may now or in the future qualify as pharmaceutically active agents for treatment of a viral hepatitis infection, and which interfere with the actions of these viral proteins, are well within the scope of the instant invention.

Other active ingredients under development for HCV therapy and potentially not falling in any of the above categories are BMS-824393, Celgosivir, A-831, KPE02001003, TCM700C, PYN-17, BIT225, JTK-652, BMS-791325 and ACH-806 (GS-9132).

### Patient Stratification According to Response to Antivirals: Non-Responders and Relapsers

A patient which does not respond to viral hepatitis treatment, and in particular to HCV treatment, may be designated as a “non-responder” or “therapy resistant” patient. A complete response is understood to refer to a decrease of the virus load below the detection limit for at least 6 months after therapy has ceased (“sustained virological response”, SVR). The therapy usually is a combination of pegylated interferon alpha and ribavirin. “Non-responders” or “therapy resistant” are patients for which no decrease of virus load by a factor of least 2 log steps is observed during 24 weeks or for which up to week 24, HCV-RNA is still detectable during therapy. The terms “therapy” and “treatment” can be used interchangeably.

A relapse refers to a complete virological response up until the 24th week of treatment at the latest. However, after the therapy has ceased, a renewed increase of virus load is observed (therapy refractory). Such patients are designated as “relapser” or “therapy refractory” patients.

A patient that is “resistant or refractory to therapy with at least one pharmaceutically active agent for treatment of a viral hepatitis infection” is a patient that has undergone therapy with at least one pharmaceutically active agent for treatment of a viral hepatitis infection, and can have undergone Standard of Care (SOC) therapy for his condition, but was either found resistant to such therapy, or who relapsed after such therapy. The at least one pharmaceutically active agent for treatment of a viral hepatitis infection to be used in the inventive methods, kits etc. for the treatment of such a patient may be the same as the one or several pharmaceutically active agents for treatment of a viral hepatitis infection which he was found resistant or refractory to, or it, or they, may be different. It should be noted here, that where it is referred to a “first pharmaceutically active agent for treatment of a viral hepatitis” or a “second pharmaceutically active agent for treatment of a viral hepatitis”, these agents are meant to differ from each other and from the proteasome inhibitor, not from the agent that a patient has shown to be resistant or refractory to.

If patients are mentioned in the context of the present invention, this term can relate to patients suffering from a hepatitis viral infection which are selected according to inclusion and exclusion criteria in accordance with the guidelines of the International Conference of Harmonization (ICH) as they are practiced e.g., by the Food an Drug Administration (FDA) or the European Medicines Agency (EMEA) for clinical trials being concerned with hepatitis viral infections. Patients may thus be of Caucasian origin, of average weight, male or female and may be 20 to 60 years of age.

### Treatment Schedules for Viral Hepatitis

Common viral hepatitis infection treatment schedules include:

Hepatitis A: dietary and lifestyle adjustments (no alcohol, reduced lipid intake) until the infection is cleared; these adjustments are usually considered to have a significant negative impact on quality of life by affected patients.

Acute Hepatitis B: symptomatic treatment (bed rest, reduction of intake of agents causing hepatic stress), until symptoms abate; in severe cases, recovery is assisted by lamivudine treatment (typically 2 mg/kg body weight, twice daily). Inactive chronic Hepatitis B: lifestyle adjustment, continuous monitoring. Active chronic Hepatitis B: administration of interferon-α (typically 5-6 Mio. Units 3 times per week), Peg-Interferon alpha-2a (typically 180 μg once per week) Peg-Interferon alpha-2b (typically 50-100 μg once per week), lamivudin (typically 100 mg daily), entecavir (typically 0.5 mg to 1 mg once daily), telbivudin (typically 600 mg once daily), or adefovir (typically 10 mg to 30 mg once daily), for at least several months and up to several years; combination therapies do apparently not improve primary outcome, but are employed if and when resistance emerges.

Hepatitis C: The standard of care (SOC) for patients with chronic hepatitis C is pegylated interferon alpha in combination with ribavirin (herein also referred to as “standard therapy” in the context of Hepatitis C treatment). Two pegylated interferons, alpha-2a (40 kD) and −2b (12 kD), are approved. The aim of antiviral therapy is the sustained elimination of the hepatitis C virus. The HCV genotype is the most important predictive factor for treatment response of patients with chronic hepatitis C and has become an important decision criterion for treatment duration and ribavirin dosage. The SOC treatment duration is 48 weeks and 24 weeks for patients infected with HCV genotype 1 and 2/3, respectively. The SOC ribavirin dosage is 1,000-1,200 mg and 800 mg for patients infected with HCV genotype 1 and 2/3, respectively. Peginterferon alpha-2a is recommended at 180 μg/week, alpha-2b at 1.5 μg/kg/week.

The initial virologic response of patients with chronic hepatitis C shows large individual variation and can be classified into rapid virologic response (HCV RNA undetectable after 4 weeks of therapy), complete early virologic response (HCV RNA undetectable after 12 weeks of therapy), partial early virologic response (≧2 log 10 decline of HCV RNA after 12 weeks of treatment but still HCV RNA positive), slow virologic response (≧2 log 10 decline of HCV RNA after 12 weeks of treatment but still HCV RNA positive followed by undetectable HCV RNA by week 24) and non-response (detectable HCV RNA 24 weeks after start of antiviral treatment). The faster a patient develops undetectable HCV RNA, the higher is his/her probability to achieve a sustained virologic response. Based on the rapid virologic response criterion, individualization of treatment duration is possible without reduction of the overall sustained virologic response rate.

Patients infected with HCV genotype 1 who start with a low baseline viral load (<600,000 IU/mL) and who achieve a rapid virologic response were shown to have favorable sustained virologic response rates after 24 weeks of antiviral treatment indicating that a shorter treatment duration can be considered in this group of patients. The possibility of shorter treatment duration was also investigated in patients with HCV genotype 2/3 infection. Smaller trials showed that a shorter treatment duration of 12-14 weeks is equally effective as the standard treatment duration in patients infected with HCV genotype 2/3 who achieve a rapid virologic response after 4 weeks of therapy. However, the large ACCELERATE trial comparing 16 versus 24 weeks of treatment in patients with HCV genotype 2/3 infection showed that a shorter treatment duration of 16 weeks results in lower sustained virologic response rates compared with the standard treatment duration. In the ACCELERATE trial, a shorter course of therapy over 16 weeks has been shown to be as effective as a 24 week course in those patients with genotype 2/3 infection who have a baseline viral load ≦400.000 IU/mL and rapid virologic response. In patients with genotype (2 and) 3 infection without a rapid virologic response (<50 IU/mL) at week 4, a longer than 24 weeks treatment duration may be necessary to optimize sustained virologic response rates.

Patients infected with HCV genotype 1 and slow virologic response have a high risk to relapse after 48 weeks of treatment with PEG-interferon and ribavirin. An approach to reduce the relapse rates is treatment extension to 72 weeks. In a German multicenter study, patients infected with HCV genotype 1 were randomized for treatment with PEG-interferon alpha-2a/ribavirin 800 mg for 48 weeks and 72 weeks, respectively. In this study, the overall sustained virologic response rate was not superior in patients infected with genotype 1 treated for 72 weeks with PEG-interferon alpha-2a/ribavirin 800 mg compared with patients treated for 48 weeks. However, the subgroup analysis of patients infected with HCV genotype 1 and a slow virologic response showed a significantly lower relapse rate in patients treated for 72 weeks compared with patients treated for 48 weeks indicating that a subgroup of patients may benefit from extended treatment duration. The SUCCESS study was the first prospective study to compare 48 weeks of treatment with 72 weeks of treatment in slow responders. In this trial slow responders were randomized at week 36 of treatment to receive PEG-interferon alpha-2b (1.5 μg/kg/week) plus weight-based dosed ribavirin (800-1400 mg/day) for a total of 48 or 72 weeks. Of the 1,427 patients included in the trial 157 (11%) were slow responders. In the intent to-treat analysis, sustained virologic rates were not different between the two groups (43% and 48% in the 48 and the 72 week treatment arms, respectively). Patients, however, who showed 80/80/80 compliance had sustained virologic response rates of 44% and 57% in the 48 weeks and 72 weeks treatment arms, respectively. Overall, these studies indicate that extended treatment duration can be considered in slow responders, however, adherence to treatment is crucially important.

The treatment options for patients with chronic hepatitis C and non-response to antiviral treatment are sparse. It was hypothesized that long term maintenance therapy with interferon may reduce progression to liver cirrhosis and its complications. In the HALT-C trial 1,050 patients with prior non response to PEG-interferon alpha/ribavirin and advanced fibrosis/cirrhosis were randomized for treatment with low dose PEG-interferon alpha-2a 90 μg/week or no treatment for 3.5 years. The primary end point was progression of liver disease defined as liver related death, hepatocellular carcinoma, hepatic decompensation or increase of the ISHAK fibrosis score of 2 or more points. The level of aminotransferases, HCV-RNA and necroinflammatory scores decreased significantly, however, there was no difference between the groups in the rate of any primary outcome. Similar results were observed in the CoPilot10 and the EPIC311 trials investigating long term PEG-interferon alpha-2b 0.5 mg/kg vs colchicine for 4 years or PEG-interferon alpha-2b 0.5 mg/kg vs no treatment in patients for 5 years with prior non-response to PEG-interferon/ribavirin, respectively.

(d) Hepatitis D: as HDV virions are incapable of replication absent an HBV infection, therapy is as given above for HBV.

(e) Hepatitis E: symptomatic therapy until symptoms abate.

(f) Hepatitis G: GB virus C (GBV-C), formerly known as Hepatitis G virus (HGV), is a virus in the Flaviviridae family which has not yet been assigned to a genus. Hepatitis G virus and GB virus C (GBV-C) are RNA viruses that were independently identified in 1995, and were subsequently found to be two isolates of the same virus. Although GBV-C was initially thought to be associated with chronic hepatitis, extensive investigation failed to identify any association between this virus and any clinical illness. So far, no therapy has been approved.

(g) Hepatitis as a complication of Epstein Barr Virus infection: Infectious mononucleosis, also known as EBV infectious mononucleosis, Pfeiffer's disease or Filatov's disease, and colloquially as kissing disease, mono (in North America) and glandular fever (in other English-speaking countries) is an infectious, very widespread viral disease caused by the Epstein-Barr virus (EBV); hepatitis occurs as a rare (<5%) complication of EBV infectious mononucleosis. Infectious mononucleosis is generally self-limiting and only symptomatic and/or supportive treatments are used (acetaminophen/paracetamol or non-steroidal anti-inflammatory drugs may be used to reduce fever and pain; Prednisone is commonly used as an anti-inflammatory to reduce symptoms of pharyngeal pain, odynophagia, or enlarged tonsils). There is little evidence to support the use of aciclovir, although it may reduce initial viral shedding. However, the antiviral drug valacyclovir has recently been shown to lower or eliminate the presence of the Epstein-Barr virus in patients afflicted with acute mononucleosis, leading to a significant decrease in the severity of symptoms

(h) Hepatitis as a complication of Cytomegalovirus infection: Cytomegalovirus is a herpes viral genus of the Herpesviruses group; in humans it is commonly known as HCMV or Human Herpesvirus 5 (HHV-5). Most healthy people who are infected by HCMV after birth have no symptoms. Some of them develop an infectious mononucleosis/glandular fever-like syndrome, with prolonged fever, and a mild hepatitis. A sore throat is common. After infection, the virus remains latent in the body for the rest of the person's life. Overt disease rarely occurs unless immunity is suppressed either by drugs, infection or old-age. Initial HCMV infection, which often is asymptomatic is followed by a prolonged, inapparent infection during which the virus resides in cells without causing detectable damage or clinical illness. However, in patients with a depressed immune system, CMV-related disease may be much more aggressive. CMV hepatitis may cause fulminant liver failure in such patients. Cytomegalovirus Immune Globulin Intravenous (Human) (CMV-IGIV), is an immunoglobulin G (IgG) containing a standardized amount of antibody to Cytomegalovirus (CMV). It may be used for the prophylaxis of cytomegalovirus disease associated with transplantation of kidney, lung, liver, pancreas, and heart. Ganciclovir treatment is used for patients with depressed immunity who have either sight-related or life-threatening illnesses. Valganciclovir may be applied effectively by orally administration, yet its therapeutic efficacy is frequently compromised by the emergence of drug-resistant virus isolates. Foscarnet or cidofovir are only given to patients with CMV resistant to ganciclovir, as, e.g., foscarnet often causes nephrotoxicity.

(i) Hepatitis as a complication of yellow fever virus infection: Yellow fever virus is a 40 to 50 nm enveloped RNA virus with positive sense of the Flaviviridae family. The virus is transmitted by the bite of mosquitos. A safe and efficient vaccine exists, yet official estimations of the WHO still amount to 200,000 cases of disease and 30,000 deaths a year. The disease presents itself in most cases with fever, nausea and pain and it disappears after several days. In some patients, a toxic phase follows, in which liver damage with jaundice (giving the name of the disease) can occur and lead to death. Currently, there is no causative cure for yellow fever. Hospitalization is advisable and intensive care may be necessary because of rapid deterioration in some cases. Different methods for acute treatment of the disease have been shown to of limited success; passive immunisation after emergence of symptoms is probably without effect. Ribavirin and other antiviral drugs as well as treatment with interferons do not have a positive effect in patients. A symptomatic treatment includes rehydration and pain relief with drugs like paracetamol.

(j) Hepatitis as a complication of mumps virus and rubella virus infection: Rare incidences of acute or fulminant hepatitis have been reported in conjunction with mumps virus and rubella virus (Matsunaga, T., et al., Journal of the Japan Pediatric Society 2003, 107:1645; Masao, A., et al., Journal of Gastroenterology 1995, 30: 539); no treatment exists or is under development.

### Further Embodiments

In the first embodiment, the instant invention relates to a kit for the treatment of a hepatitis viral infection in a human or animal individual who does not respond or is refractory to treatment with at least one pharmaceutically active agent for treatment of a viral hepatitis infection, comprising: at least one first pharmaceutical container comprising a pharmaceutical composition comprising at least one proteasome inhibitor; and at least one second pharmaceutical container comprising a pharmaceutical composition comprising at least one first pharmaceutically active agent for treatment of a viral hepatitis infection, and optionally at least one second pharmaceutically active agent for treatment of a viral hepatitis infection, comprised in the composition of said at least one second or in at least one third pharmaceutical container.

In the second embodiment, the invention relates to a use of at least one proteasome inhibitor and at least one first pharmaceutically active agent for treatment of a viral hepatitis infection, and optionally at least one second pharmaceutically active agent for treatment of a viral hepatitis infection, in the manufacture of a pharmaceutical composition for the treatment of a hepatitis viral infection in a human or animal individual who does not respond or is refractory to treatment with at least one pharmaceutically active agent for treatment of a viral hepatitis infection.

In a third embodiment, the invention relates to pharmaceutical compositions essentially as provided by the second embodiment's uses described herein.

In fourth embodiment, the invention relates to a pharmaceutical composition for the treatment of a hepatitis viral infection in a human or animal individual who does not respond or is refractory to treatment with at least one pharmaceutically active agent for treatment of a viral hepatitis infection, comprising at least one proteasome inhibitor being selected from PS-341 and S-2209 and at least one first pharmaceutically active agent for treatment of a viral hepatitis infection, wherein said at least one first pharmaceutically active agent for treatment of a viral hepatitis infection is pegylated interferon-alpha, and said at least one second pharmaceutically active agent for treatment of a viral hepatitis infection, where present, is ribavirin.

In a fifth embodiment, the invention relates to a method of treating a hepatitis viral infection in a human or animal individual who does not respond or is refractory to treatment with at least one pharmaceutically active agent for treatment of a viral hepatitis infection, comprising the step of administering to such individual at least one kit of pharmaceutical compositions as provided herein, or at least one pharmaceutical composition as provided herein.

In a sixth embodiment, the invention relates to a combination of at least one proteasome inhibitor, at least one first pharmaceutically active agent for treatment of a viral hepatitis infection, and optionally at least one second pharmaceutically active agent for treatment of a viral hepatitis infection, for the treatment of a hepatitis viral infection in a human or animal individual who does not respond or is refractory to treatment with at least one pharmaceutically active agent for treatment of a viral hepatitis infection, essentially as provided by the first embodiment's kits of pharmaceutical compositions described herein before, or the second embodiment's uses described herein before.

In a seventh embodiment, the invention relates to the novel kits, compositions, methods and uses essentially as provided by the first through sixth embodiment described herein before.

In these and other embodiments, specific proteasome inhibitors such as S-2209, PS-519, PS-341 (Bortezomib) and PS-273 can be used, interferons including their derivatives such as pegylated interferon alpha as first pharmaceutically active agent and nucleoside or nucleotide analogs such as ribavirin as second pharmaceutically active agent. These may be the sole pharmaceutically active agents comprised within the pharmaceutical compositions and kits.

Therein, the interferon can be an interferon preparation chosen from the list of: albinterferon, PEG-IFN alpha-2a, alpha-2b or lambda, Locteron, Omega IFN, Medusa IFN, DA-3021, EMZ702, Infradure, IL-28, IL-29, Veldona, Soluferon and Belerofon. The interferon may also be replaced by, or assisted by, additional administration of, for example, an interleukin, such as IL-1, IL-2, IL-6, IL-7, IL-8, IL-12, IL-15, IL-18, IL-21, IL-22, IL-28, and IL29, a steroid, or an immunomodulator, e.g., Thymosin, ME3738, SCV-07, Alinia, Oglufanide, IPH-1101, CYT 107, or EGS-21, ciclosporin and derivatives thereof. The first pharmaceutically active agent for treatment of a viral hepatitis infection can be, for example, one that stimulates or assists the body's own functions, such as the body's natural defenses, such as against viruses.

Similarly, the second pharmaceutically active agent may be one of, or any combination of the elements of the following list: telaprevir, boceprevir, ITMN-191, SCH 900518, TMC435, BI201335, MK-7009, lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, thymosin alpha 1, a vaccine, glycyrrhizin, ciclosporin and derivatives thereof, e.g., SCY-635, DEBIO-025, NIM811, Silibinin, Nitazoxanide, A-831, KPE02001003, TCM700C, PYN-17, BIT225, JTK-652, BMS-791325, ACH-806 (GS-9132), amantadine, rimantadine and derivatives thereof, and azathoprine.

The PROVE2 and PROVE3 studies referenced above aptly demonstrated that a combination of, for example, standard therapy including interferon+ribavirin with, for example, telaprevir provides an additional benefit over standard therapy or interferon+telaprevir only. The rapid development of resistances against single therapeutic compounds in hepatitis-causing viruses necessitates a multi-pronged approach if elimination of the pathologic agent is to be achieved. Therefore, the inventive pharmaceutical composition can comprise at least two of the second pharmaceutically active agents for treatment of a viral hepatitis infection, or at least 3, or at least 4 of the second pharmaceutically active agents for treatment of a viral hepatitis infection.

Hence, the inventive pharmaceutical compositions and kits can comprise a total of at least 2, or at least 3, yet or at least 4, or at least 5, or at least 6, or at least 7 pharmaceutically active ingredients.

The pharmaceutical compositions and kits may be used for treating patients suffering from viral hepatitis and in particular from HCV infections. It can be advantageous to use the pharmaceutical compositions and kits for treating patients suffering from viral hepatitis and in particular from HCV infections which are considered as “non-responding” or “refractory” patients.

One advantage of the kits in accordance with the present invention is that they contain the pharmaceutical containers of compositions in separate form, e.g., as different solutions, tablets etc. This may allow for a timely ordered, i.e. subsequent administration of the compositions of separate pharmaceutical containers which can be important when treating “non-responding” or “refractory” patients suffering e.g., from HCV infections.

The kits in accordance with the present invention may thus comprise instructions in paper or electronic form advising the user to first administer the compositions of the first pharmaceutical container comprising the proteasome inhibitor and to administer the compositions of the second and/or third pharmaceutical containers subsequently with delay. Alternatively, they may comprise instructions in paper or electronic form advising the user to first administer the proteasome inhibitor and to administer the first and optionally the second pharmaceutically active agent for treatment of a viral hepatitis infection subsequently after a delay. Conversely, the instructions may instruct the user first to administer the composition of the second and optional third pharmaceutical container(s) comprising the first and optional second pharmaceutically active agent for treatment of a viral hepatitis infection, and to administer the compositions of the first pharmaceutical container comprising the proteasome inhibitor subsequently with delay, or to first administer the compositions of the second and/or third pharmaceutical container, and administer the proteasome inhibitor subsequently with delay.

The instructions may further advise the user on a specified time period for the delay between administering the first pharmaceutical composition and administering the second and/or third pharmaceutical composition, in either sequence.

The instructions may thus specify such a specified time period of about 1 day, about 2 days, about 2 to 4 days, about 4 to 6 days, about 1 week, about 1 to 2 weeks, about 2 to 3 weeks, about 2 to 4 weeks, about 2 to 5 weeks, about 2 to 6 weeks, about 2 to 7 weeks, about 2 to 8 weeks, about 3 to 4 weeks, about 3 to 5 weeks, about 3 to 6 weeks, about 3 to 7 weeks, about 3 to 8 weeks, about 4 to 5 weeks, about 4 to 6 weeks, about 4 to 7 weeks, about 4 to 8 weeks, about 4 to 10 weeks, about 4 to 12 weeks, about 6 to 8 weeks, about 6 to 10 weeks, or about 6 to 12 weeks; or more than about 2 weeks, more than about 3 weeks, more than about 4 weeks, more than about 5 weeks, more than about 6 weeks, more than about 7 weeks, more than about 8 weeks, more than about 10 weeks, or more than about 12 weeks and/or less than about 4 weeks, less than about 5 weeks, more less 6 weeks, less than about 7 weeks, less than about 8 weeks, less than about 10 weeks, or less than about 12 weeks, or any other combination not yet expressly given above of these upper and lower limits, after treatment with the proteasome inhibitor has ended and before commencing treatment with the second and/or third pharmaceutical composition.

Moreover, in other embodiments of the instant invention, e.g., where the inventive pharmaceutical compositions, methods and combinations are concerned, certain compositions may be provided in a manner such that certain treatment regimes can be followed, e.g., the administration of the proteasome inhibitor concurrent with, before, or subsequent to the administration of the first and optional second pharmaceutically active agent for treatment of a viral hepatitis infection. Where the proteasome inhibitor is to be provided in a manner such that it is administrated before or subsequent to the administration of the first and optional second pharmaceutically active agent for treatment of a viral hepatitis infection, it can be provided such that a specified time period for the delay between these administrations is, or may be, observed. This may advantageously be achieved by providing the proteasome inhibitor and the first and optional second pharmaceutically active agent for treatment of a viral hepatitis infection in separate formulations, and by optionally packaging or providing with these separate formulations instructions in written or electronic form detailing the procedure, including detailing the specified time period for the delay. The specified time periods for the delay can be the same or similar for these embodiment as those described for the inventive kits above.

In a specific embodiment, the delay is more than about two weeks, but less than about 8 weeks. In the clinical trial, the results of which are presented herewith, the range of time intervals between the end of the proteasome inhibitor treatment and the commencement of standard therapy (delay to retreatment) was between 3 and 7.6 weeks. Only the patient with the longest delay of 7.6 weeks experienced a more delayed decline in HCV RNA copy numbers in his blood. This result suggests that the optimal delay to retreatment may be more than about two weeks, but less than about 8 weeks.

The instructions may additionally advise that treatment with the proteasome inhibitor may on a three to four daily basis such as e.g., on day 1, 4, 8 and 11, or on day 1, 4, 7, 10 or on day 1, 5, 9 and 13 and the like.

Instructions for treatment with the compositions of the second and/or third pharmaceutical container(s) may follow the dosage regimen developed for these containers. In case of an interferon such as PEG-interferon-alpha and a nucleoside analog such as ribavirin, treatment may be undertaken on a one to two weekly basis for 32 to 60 weeks such as 48 weeks in case of infections with HCV genotype 1 and on a one to two weekly basis for 12 to 36 weeks such as 24 weeks in case of infections with HCV genotype 2/3.

The pharmaceutical compositions may be formulated for oral, subcutaneous, transdermal, rectal, peritoneal or intravenous administration and may contain suitable pharmaceutically acceptable excipients.

If only a first pharmaceutically active agent for treatment of a viral hepatitis infection is present, this can be selected from interferons including their derivatives such as pegylated interferon alpha as first pharmaceutically active agent or from nucleoside analogs such as ribavirin. If, however, at least two pharmaceutically active agents are present, these can be selected from interferons including their derivatives such as pegylated interferon alpha as first pharmaceutically active agent and from target specific antivirals, e.g., nucleoside analogs, such as ribavirin, as second pharmaceutically active agent.

According to the invention, the treatment with a proteasome inhibitor may be initiated in patients who received therapy for a viral hepatitis infection, and in particular for a HCV infection, such as interferons including their derivatives and/or nucleoside analogs, and which have been classified as non-responders or refractory.

In certain embodiments, the patient can be any patient infected with, or at risk for infection with, a virus inducing hepatitis, and specifically HCV. Infection or risk for infection can be determined according to any technique deemed suitable by the practitioner of skill in the art. In one embodiment, patients are humans infected with HCV.

In certain embodiments, specifically of the eighth to twenty-ninth embodiment, the patient has never received therapy or prophylaxis for a virally induced hepatitis, or more particularly an HCV infection.

In further embodiments, the patient has previously received therapy or prophylaxis for a virally induced hepatitis, or more particularly an HCV infection. For instance, in certain embodiments, the patient has not responded to treatment for a virally induced hepatitis, or more particularly an HCV infection. In certain embodiments, the patient can be a patient that received therapy but continued to suffer from viral infection or one or more symptoms thereof. In certain embodiments, the patient can be a patient that received therapy but failed to achieve a sustained virologic response. In certain embodiments, the patient has received therapy for a virally induced hepatitis, or more particularly a hepatitis induced by HCV infection, but has failed to show, for example, a 2 log 10 decline in viral RNA levels after 12 weeks of therapy.

In certain embodiments, the patient is a patient that discontinued therapy for a virally induced hepatitis, or more particularly a hepatitis induced by HCV infection, because of one or more adverse events associated with the therapy. The patient may also be a patient that is unwilling to, or refuses to, take up therapy, or re-therapy, potentially due to his or her expectation of grave side effects.

The most frequently observed side effects of an IFN therapy are flu-like symptoms such as fever, headache, muscle pain, joint pain as well as fatigue, loss of appetite and loss of weight. Moreover, neuropsychiatric side effects including mood swings, insomnia, anxiety, depression, psychosis, suicidal ideation, actual suicide and homicide have been described. Pegylated interferon may also induce autoimmune disorders, or may worsen preexisting autoimmune disorders, e.g., autoimmune thyroiditis. A frequent side effect observed with ribavirin treatment is anemia, particularly hemolytic anemia, which necessitates continuous control of blood parameters during therapy. Other ribavirin-associated adverse events include mild lymphopenia, hyperuricemia, itching, rash, cough, and nasal stuffiness. In addition, it has caused fetal death and fetal abnormalities in animals, making the use of contraceptive measures imperative in female patients receiving ribavirin treatment. Therapy related adverse events are a major reason for patients discontinuing or outwardly refusing therapy. For instance, psychiatric side effects common with interferon therapy are responsible for about 10% to 20% of discontinuations of current therapy for HCV infection.

Hence, in certain embodiments, the patient is a patient where current therapy is not indicated. In fact, quite a number of HCV-infected individuals are ineligible to receive SOC-treatment for Hepatitis C Virus infection due to them presenting with characteristics currently contraindicated for such therapy. These include uncontrolled depressive illness, organ transplants, autoimmune conditions known to be exacerbated by IFN and/or ribavirin, untreated thyroid disease, pregnancy or refusal to practice contraception, severe hypertension, significant coronary heart disease or heart failure, poorly controlled diabetes, COPD, or an age of less than 2 years.

The kits, methods and compositions provided herein may reduce or eliminate the need for exposing patients to the agents of current therapy, either by reducing the dose needed or reducing the required time of exposure to these agents, or by facilitating the replacement of certain agents of current therapy.

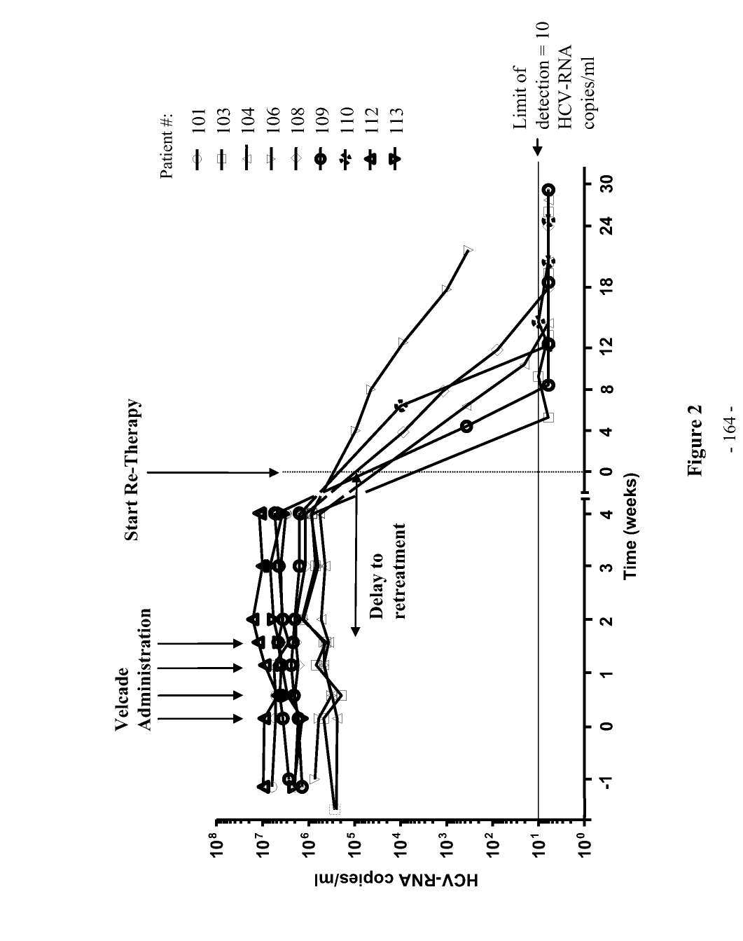

In the small clinical trial, the results of which are presented herewith, it was demonstrated that a very short course of treatment with a proteasome inhibitor (4 doses over 11 days, corresponding to a single round of treatment in the therapy of multiple myeloma; for multiple myeloma, up to 9 such rounds are administered) followed by standard of care therapy for HCV infection, led to a very rapid decline (within 4 to 12 weeks) in copy numbers of infectious Hepatitis C Virus particles in patients' blood after reuptake of standard therapy several weeks post termination of the proteasome inhibitor treatment (see FIG. 2). This was despite the fact that the proteasome inhibitor employed in this study possesses a half life on the order of hours to days, and was therefore cleared from the bloodstream long before standard therapy was recommenced. It seems hence more than likely that the present inventive methods allow for a modification of the methods of standard therapy, towards reduced exposure to the agents of standard therapy. The associated reduction in side effect occurrence and/or severity can enable certain patients previously ineligible for, or unwilling to undergo, therapy to reap the benefits of the modified therapeutic regimens.

Accordingly, provided are methods of treating or preventing a virally induced hepatitis, or more particularly a hepatitis induced by HCV infection, in patients where the risk of neuropsychiatric events, such as depression, contraindicates treatment with current therapies. In one embodiment, provided are methods of treating or preventing a virally induced hepatitis, or more particularly a hepatitis induced by HCV infection, in patients where a neuropsychiatric event, such as depression, or risk of such indicates discontinuation of treatment with current therapy. Further provided are methods of treating a virally induced hepatitis, or more particularly a hepatitis induced by HCV infection, in patients where a neuropsychiatric event, such as depression, or risk of such indicates dose reduction, or reduction of time of exposure to, current HCV therapy.

Current therapy is also contraindicated in patients that are hypersensitive to interferon or ribavirin, or both, or any other component of a pharmaceutical product for administration of interferon or ribavirin. Current therapy is not indicated in patients with hemoglobinopathies (e.g., thalassemia major, sickle-cell anemia) and other patients at risk from the hematologic side effects of current therapy. Common hematologic side effects include bone marrow suppression, neutropenia and thrombocytopenia. Furthermore, ribavirin is toxic to red blood cells and is associated with hemolysis. Accordingly, in one embodiment, provided are methods of treating or preventing a virally induced hepatitis, or more particularly a hepatitis induced by HCV infection, in patients hypersensitive to interferon or ribavirin, or both, patients with a hemoglobinopathy, for instance thalassemia major patients and sickle-cell anemia patients, and other patients at risk from the hematologic side effects of current therapy.

In certain embodiments, the patient has received treatment for a virally induced hepatitis, or more particularly a hepatitis induced by HCV infection, and discontinued that therapy prior to administration of a method provided herein. In further embodiments, the patient has received therapy and continues to receive that therapy along with administration of a method provided herein.

Treatment with at least one proteasome inhibitor may include concentrations of the proteasome inhibitors used within the range of about 1 nM to about 50 μM, or about 10 nM to about 10 μM in the pharmaceutical composition. The proteasome inhibitors may be used at doses of about 0.25 to about 5, of about 0.4 to about 2.5, or of about 0.7 to about 1.5 mg/m² body surface. Such treatment can result in plasma concentrations of the respective compound(s) in a range of, e.g., 1 nM to 100 nM, or 5 nM to 500 nM, or 10 nM to 1 μM, or 50 nM to 5 μM, or 10 nM to 100 nM, or 50 nM to 500 nM, or 100 nM to 1 μM, or 500 nM to 5 μM, or 1 μM to 10 μM. As a first approximation, the plasma concentration may be assumed to be similar (i.e. within a factor of e.g., 1.5, 2, 3, or 4) to the concentration of the respective compound in the target tissue and around the target cells.

Treatment with proteasome inhibitors may be performed over several days and weeks up to months. Usually a proteasome inhibitor may be administered every day, or every second day, or every third day, or twice a week, or once a week, or once every two weeks, or once every month.

In some embodiments, the number of times the proteasome inhibitor needs to be administered is limited. For example, a single round of treatment with the proteasome inhibitor may encompass not more than 20, not more than 15, not more than 10, not more than 8, not more than 6, not more than 4, not more than 2, or not more than a single administration of the proteasome inhibitor. Some embodiments provide not more than 3, or not more than 10, administrations. In another embodiment, a round of treatment encompasses 2 to 20, or 2 to 15, or 2 to 10, or 4 to 10, or 3 to 10, or 4 to 8 administrations. In a specific embodiment, the range is 3 to 10 administrations. Moreover, not more than 8, not more than 5, not more than 4, not more than 3, not more than 2, and or only a single round of treatment is necessary to achieve the desired therapeutic effect. In another embodiment, the range is 1 to 8, 1 to 5, 1 to 4, 1 to 3, or 1 to 2. The range of 1 to 2 is specifically contemplated. These limitations may optionally be spelled out in instructions in paper or electronic form which accompany the packaged form of the proteasome inhibitor.

Subsequent to said treatment with a proteasome inhibitor, administration of the at least first and optionally of the at least second pharmaceutically active agent is commenced. In one embodiment one may initiate a treatment (after proteasome inhibitor treatment) which is identical to the therapy for which the patient has been classified as non-responder or refractory. However, the new treatment may also differ from the previous treatment.

For example, if a patient has shown no response or a refractory response towards treatment with interferons and nucleoside analogs, one may treat this patient (after he has received proteasome inhibitor treatment) again with interferons and nucleoside analogs.

However, if for example a patient has received interferons only, he may also receive nucleoside analogs or a combination of nucleoside analogs and interferons after the proteasome inhibitor treatment.

The treatment with a first and optionally with a second pharmaceutically active agent for treatment of a viral hepatitis infection may follow the treatment with the proteasome inhibitor either rather directly (e.g., the following day) or after a break (i.e. a therapy free period) of one to several days or weeks, such as 1 week, 2 weeks, 3 weeks, 4 weeks, 5 weeks, 6 weeks, more than 6 weeks etc. Starting with the therapy after a break of about 1, 2, 3, 4, 5, or 6 weeks may be recommendable in order to await whether the proteasome inhibitor alone improved the patient's condition. For further details on a delay between the administration of the proteasome inhibitor and the pharmaceutically active agent, or agents, for treatment of a viral hepatitis infection, please see elsewhere herein.

Further, the treatment with a first and optionally with a second pharmaceutically active agent for treatment of a viral hepatitis infection may be re-commenced with the same frequency and dosage as it would have been performed without the patients receiving proteasome inhibitors. This may, or may not, entail a re-assessment of the dosing regimen for the patient, based on changes in body weight, body composition, overall health status, co-morbidities etc. that may have precipitated since the patient was last assessed for purposes of establishing a dose for standard HCV therapy. Proceeding in this manner can be advantageous.

If e.g., proteasome specific inhibitors are applied during treatment of a viral Hepatitis C infection and if this treatment is followed by standard therapy with pegylated interferons and ribavirine, treatment with pegylated interferons and ribavirine may be undertaken on a two weekly basis for 48 weeks in case of infections with HCV genotype 1 and on a two weekly basis for 24 weeks in case of infections with HCV genotype 2/3.

The clinical study described below revealed that no significant reduction of virus load could be observed after administering the proteasome inhibitor PS-341 (Bortezomib, Velcade®) to standard therapy-resistant/-refractory HCV-infected patients. However, after receiving proteasome inhibitors and being treated again with the standard therapy (PEG-IFN plus ribavirine), patients showed a significantly lower virus load. In several cases, the HCV viral load dropped below the limit of detection.

Based on these results it is justified to assume that a similar effect may be observed if a combination of at least one proteasome inhibitor together with at least one other pharmaceutically active agent which is known to be effective in treating viral hepatitis is administered simultaneously.

That concurrent treatment with a proteasome inhibitor and at least one first pharmaceutically active agent for treatment of a viral hepatitis infection may be as effective as sequential treatment with the proteasome inhibitor in the lead may be indicated by the results of the in vitro experiments presented herewith. Therein, both the application of proteasome inhibitor first, followed by a treatment free period, followed by application of at least one pharmaceutically active agent for treatment of a viral hepatitis infection, and two concurrent applications of proteasome inhibitor and at least one pharmaceutically active agent for treatment of a viral hepatitis infection at 24 hour intervals, were more effective in reducing viral replication than applying the at least one pharmaceutically active agent for treatment of a viral hepatitis infection, followed by the proteasome inhibitor.

The active agent(s) and the proteasome inhibitor can be provided in such manner as to allow for a convenient application of the respective compound in the manner envisaged. E.g., where simultaneous administration is considered, the pharmaceutically active agent(s) for treatment of a viral hepatitis infection and the proteasome inhibitor may be formulated together, while for subsequent treatment, separate formulation may be more advantageous.

In another embodiment, the present invention thus relates to the use of at least one proteasome inhibitor together with at least one pharmaceutically active agent for treatment of a viral hepatitis infection in the manufacture of a medicament for treating patients which do not respond or are refractory to treatment with a pharmaceutically active agent for treatment of a viral hepatitis infection alone. In this and other embodiments, where the term “alone” is used it is meant to indicate the lack of a proteasome inhibitor, not that only a single pharmaceutically active agent was used in the treatment of the patient when he or she was found non-respondent or refractory. On the contrary, most of these patients will have been treated with the SoC combination of IFN/Ribavirin, and found non-respondent or refractory to this combination.

Yet another embodiment of the present invention relates to the use of at least one proteasome inhibitor together with at least one first and at least one second pharmaceutically active agent for treatment of a viral hepatitis infection in the manufacture of a medicament for treating patients which do not respond or are refractory to treatment with at least one pharmaceutically active agent for treatment of a viral hepatitis infection alone.

In these embodiments, specific proteasome inhibitors such as PS-519, PS-341 (Bortezomib), PS-273, and S-2209 can be used. If only one additional pharmaceutically active agent for treatment of a viral hepatitis infection is present, this can be selected from interferons including their derivatives such as pegylated interferon alpha as first pharmaceutically active agent or from nucleoside analogs such as ribavirin as second pharmaceutically active agent. If, however, at least two additional pharmaceutically active agent for treatment of a viral hepatitis infections are present, these can be selected from interferons including their derivatives such as pegylated interferon alpha as first pharmaceutically active agent and from nucleoside analogs such as ribavirin as second pharmaceutically active agent.

With the above mentioned pharmaceutical compositions, kits, uses and methods, in particular where they relate to a combination of a proteasome inhibitor, an interferon and a nucleoside analog, it is possible to reduce the virus load in chronic HCV patients (over several orders of magnitude) or to even completely remove the virus.

In any of the embodiments of the present invention (kits, uses, pharmaceutical compositions, methods of treatment), the proteasome inhibitor may be selected from the list of: peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (also known as aclarubicine), celastrol, withaferin A, Gliotoxin, epipolythiodioxo-piperazines, green tea polyphenolic catechins such as (−)-epigallocatechin-3-gallate, Disulfuram, acridine derivatives including tetra-acridine derivatives with betulinic acid such as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, its endocyclic oxindole-phenyl clamp (BIA-1a) and endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal, its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer “PS-293”, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209 and its structural analogs Compound I to Compound 6, and Compound 8, as provided in Leban, J., et al., Bioorg. Med. Chem. 2008, 16:4579. The at least first and second pharmaceutically active agent for treatment of a viral hepatitis infection may be pegylated interferon alpha and ribavirin.

With the administration of a proteasome inhibitor, it may be possible to advantageously influence even the efficacy of the standard of care treatment, or at any rate the treatment with said first and optionally second pharmaceutically active agent for treatment of a viral hepatitis infection for treatment of a viral hepatitis infection. For example, said first and optionally second pharmaceutically active agent for treatment of a viral hepatitis infection for treatment of a viral hepatitis infection may be administered for a reduced duration compared to the duration otherwise recommended for the respective patient under the respective circumstances for the treatment of the hepatitis viral infection with the respective agents for treatment of a viral hepatitis infection, e.g., a duration of not more than about 95%, 90% 85%, 80%, 75% 70%, 66%, 50%, 40%, 33%, 25% 15% or 10% of the recommended duration. E.g., the treatment with pegylated interferons and/or ribavirin may be continued for only about 36 weeks, instead of 48 weeks, or about 30 weeks, or about 24 weeks, or about 20 weeks, or about 15 weeks, or about 12 weeks, or about 10 weeks, or about 8 weeks, or about 6 weeks, or about 4 weeks, to achieve the desired effect. Such shortened treatment would greatly ease the discomfort felt by patients on therapies of such duration which are associated with side effects as potentially severe as those associated with long term interferon and/or ribavirin administration.

In a specific embodiment, the treatment with said first and second pharmaceutically active agent for treatment of a viral hepatitis infection is continued for only about 50% of the duration of the treatment recommended without the proteasome inhibitor, or on a two weekly basis about 24 weeks in case of infections with HCV genotype 1 and on a two weekly basis for about 12 weeks in case of infections with HCV genotype 2/3 for the presently recommended interferon/ribavirin treatment. In one embodiment, the treatment with said first and optionally second pharmaceutically active agent for treatment of a viral hepatitis infection is continued for only about 25% of the duration of the treatment recommended without the proteasome inhibitor, or on a two weekly basis about 12 weeks in case of infections with HCV genotype 1 and on a two weekly basis for about 6 weeks in case of infections with HCV genotype 2/3 for interferon/ribavirin treatment.

Instead of, or at the same time as, reducing the time of treatment necessary to achieve the desired effect, the proteasome inhibitor treatment may enable the physician to reduce the dose of said first and/or second pharmaceutically active agent for treatment of a viral hepatitis infection. For example, the treatment with said first and second pharmaceutically active agent for treatment of a viral hepatitis infection may involve administration of a reduced dose compared to the dose otherwise recommended for the respective agent in the treatment of a hepatitis viral infection with the respective agents for treatment of a viral hepatitis infection for the respective patient under the respective circumstances, e.g., not more than about 95%, 90% 85%, 80%, 75% 70%, 66%, 50%, 40%, 33%, 25% 15% or 10% of the recommended dose. In a specific embodiment, not more than about 66% of the recommended dose of at least one of the first and second pharmaceutically active agent for treatment of a viral hepatitis infection is administered. E.g., the treatment with pegylated interferons and ribavirin may only require doses of about 900, 800, 700, 600, 500, 400, 300, 200 or 100 mg ribavirin, depending on patient need and serotype, or about 140, 120, 100 80, 60, 40 or 20 μg/week Peginterferon alpha-2a, or about 1.2, 1, 0.8, 0.6, 0.4 or 0.2 μg/kg/week Peginterferon alpha-2b, to achieve the desired effect. In a specific embodiment, the dose is 66% of the recommended dose. Such treatment with reduced doses could equally ease the discomfort felt by patients on such therapy.

While 50% of the recommended duration and 66% of the recommended dose are suitable embodiments, these are primarily directed at the goal of enabling a patient to undergo treatment he or she is otherwise ineligible for, or which is otherwise too unpleasant to undergo. This may be achieved already at lesser, or may need a higher, degree of reduction in the duration and or dose. It is therefore suitable in the context of the instant invention, that a level of dose/duration reduction is established, which leads to a significant improvement in the side effect profile compared to standard therapy, whereby certain patients previously ineligible for, or unwilling to undergo, standard of care treatment, are enabled to undergo an inventive treatment as provided herein.

These limitations may optionally be spelled out in instructions in paper or electronic form which accompany the packaged form of the proteasome inhibitor, as further provided for the inventive kits herein.

### Pharmaceutical Compositions and Methods of Administration

The proteasome inhibitors can be formulated into pharmaceutical compositions using methods available in the art and those disclosed herein. Such compounds can be used in some embodiments to enhance delivery of the drug to the liver.

The methods provided herein encompass administering pharmaceutical compositions containing at least one proteasome inhibitor as described herein, with one or more compatible and pharmaceutically acceptable carriers, such as diluents or adjuvants, and/or with at least one pharmaceutically active agent for treatment of a viral hepatitis infection.

In certain embodiments, the at least one pharmaceutically active agent for treatment of a viral hepatitis infection, or the first and/or second pharmaceutically active agent for treatment of a viral hepatitis infection, can be formulated or packaged with the proteasome inhibitor. Of course, the aforesaid agent(s) will only be formulated with the proteasome inhibitor when, according to the judgment of those of skill in the art, such co-formulation should not interfere with the activity of either agent or the method of administration. In certain embodiments, the proteasome inhibitor and the aforesaid agent(s) for treatment of a viral hepatitis infection are formulated separately. They can be packaged together, or packaged separately, for the convenience of the practitioner of skill in the art.

In clinical practice the active agents provided herein may be administered by any conventional route, in particular orally, parenterally, rectally or by inhalation (e.g., in the form of aerosols). In certain embodiments, the proteasome inhibitor is administered orally.

The pharmaceutical compositions can be, for example, solid compositions for oral administration, of tablets, pills, hard gelatin capsules, powders or granules. In these compositions, the active product is mixed with one or more inert diluents or adjuvants, such as sucrose, lactose or starch.

These compositions can comprise substances other than diluents, for example a lubricant, such as magnesium stearate, or a coating intended for controlled release.

The pharmaceutical compositions can be, for example, liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening or flavoring products.

The compositions for parenteral administration can be emulsions or sterile solutions. The pharmaceutical compositions can comprise a solvent or a vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing and stabilizing agents. Sterilization can be carried out in several ways, for example using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in apyrogenic sterile water, in saline or any other pharmaceutically acceptable vehicle. For use in the form of dry aerosols intended to be directly inhaled, the active principle is finely divided and combined with a water-soluble solid diluent or vehicle, for example dextran, mannitol or lactose.

In one embodiment, a composition provided herein is a pharmaceutical composition or a single unit dosage form. Pharmaceutical compositions and single unit dosage forms provided herein comprise a therapeutically effective amount of one or more therapeutic agents (e.g., a proteasome inhibitor, or other therapeutic agent), and a typically one or more pharmaceutically acceptable carriers or excipients. In a specific embodiment and in this context, the term “pharmaceutically acceptable” means approved by a regulatory agency, e.g., of the Federal or a state government of the United States of America (e.g., FDA), and/or by a regulatory agency of a Federal government of a member state of the European Union, and/or by the European Medicines Agency (EMEA), or listed in the U.S. Pharmacopeia or other generally recognized pharmacopeia for use in animals, and more particularly in humans.

The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martinm.

Typical pharmaceutical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmocopia (USP)SP(XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre-gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, N.Y., 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a therapeutically effective amount of a therapeutic agent, in certain embodiments, in purified form, together with a suitable amount of carrier so as to provide the form for proper administration to the patient. The formulation should suit the mode of administration. In a certain embodiment, the pharmaceutical compositions or single unit dosage forms are sterile and in suitable form for administration to a patient, for example, an animal patient, such as a mammalian patient, for example, a human patient.

A pharmaceutical composition is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non aqueous liquid suspensions, oil in water emulsions, or a water in oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms provided herein will typically vary depending on their use. For example, a dosage form used in the initial treatment of viral infection may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms encompassed herein will vary from one another will be readily apparent to those skilled in the art. See, e.g., Remington's Pharmaceutical Sciences, 20th ed., Mack Publishing, Easton Pa. (2000).

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms comprising a proteasome inhibitor or pharmaceutically active agent for treatment of a viral hepatitis infection, or a pharmaceutically acceptable salt, solvate or hydrate thereof, lie within the range of from about 0.1 mg to about 1000 mg per day, given as a single once-a-day dose in the morning or as divided doses throughout the day taken with or without food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active compound.

The proteasome inhibitors and/or pharmaceutically active agents for treatment of a viral hepatitis may be formulated in their free acid or base form, if any, or they may be formulated as pharmaceutically acceptable salts of the parent compounds.

“Pharmaceutically acceptable salts” refer to derivatives of compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

### Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In certain embodiments, the oral dosage forms are solid and prepared under anhydrous conditions with anhydrous ingredients, as described in detail in the sections above. However, the scope of the compositions provided herein extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions is typically present in from about 50 to about 99 weight percent of the pharmaceutical composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms include, but are not limited to, agar, alginic acid, calcium carbonate, microcrystalline cellulose, croscarmnellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Delayed Release Dosage Forms

Active ingredients such as the proteasome inhibitors and pharmaceutically active agents provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Thus encompassed herein are single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased patient compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the drug may be administered using intravenous infusion, an implantable osmotic pump, a transdermal patch, liposomes, or other modes of administration. In one embodiment, a pump may be used (see, Sefton, CRC Crit. Ref. Biomed. Eng. 1987, 14:201; Buchwald et al., Surgery 1980, 88:507; Saudek et al., N. Engl. J. Med. 1989, 321:574). In another embodiment, polymeric materials can be used. In yet another embodiment, a controlled release system can be placed in a patient at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 1990, 249:1527). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the patient.

### Parenteral Dosage Forms

In one embodiment, a pharmaceutical compositions described herein a parenteral dosage form. Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are typically, sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non aqueous vehicles such as, but not limited to, corn oil, cottonseed oil, peanut oil, sesame oil, ethyl oleate, isopropyl myristate, and benzyl benzoate. Compounds that increase the solubility of one or more of the active ingredients disclosed herein can also be incorporated into the parenteral dosage forms.

### Transdermal, Topical & Mucosal Dosage Forms

In some embodiments, a pharmaceutical compositions described herein is a transdermal, topical, or a mucosal dosage form. Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th, 18th and 20th eds., Mack Publishing, Easton Pa. (1980, 1990 & 2000); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are non toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th, 18th and 20th eds., Mack Publishing, Easton Pa. (1980, 1990 & 2000).

Depending on the specific tissue to be treated, additional components may be used prior to, in conjunction with, or subsequent to treatment with active ingredients provided. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

### Dosage and Unit Dosage Forms

In human therapeutics, the doctor will determine the posology which he considers most appropriate according to a preventive or curative treatment and according to the age, weight, stage of the infection and other factors specific to the patient to be treated. In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated.

In further aspects, provided are methods of treating a viral hepatitis in a patient by administering, to a patient in need thereof, inter alia an effective amount of a proteasome inhibitor, or a pharmaceutically acceptable salt thereof. The amount of the compound or composition which will be effective in the prevention or treatment of a disorder or one or more symptoms thereof will vary with the nature and severity of the disease or condition, and the route by which the active ingredient is administered. The frequency and dosage will also vary according to factors specific for each patient depending on the specific therapy administered, the severity of the disorder, disease, or condition, the route of administration, as well as age, body, weight, response, and the past medical history of the patient. Effective doses may be extrapolated from dose-response curves derived from in vitro or animal model test systems.

In certain embodiments, exemplary doses of a composition include milligram or microgram amounts of the active compound per kilogram of patient or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in certain embodiments, the dosage administered to a patient is 0.140 mg/kg to 3 mg/kg of the patient's body weight, based on weight of the active compound. In certain embodiments, the dosage administered to a patient is between 0.20 mg/kg and 2.00 mg/kg, or between 0.30 mg/kg and 1.50 mg/kg of the patient's body weight. Alternatively, an estimate of the surface area of the patient's body may be used to scale the dose, as the surface area is sometimes a more accurate predictor of certain properties related to drug distribution and clearance (see, for example, Pinkel, D., Cancer Res. 1958, 18:853). The proteasome inhibitors may be used, for example, at doses of about 0.25 to about 5, of about 0.4 to about 2.5, or of about 0.7 to about 1.5 mg/m² body surface.

In certain embodiments, the recommended daily dose range of a composition provided herein for the conditions described herein lie within the range of from about 0.1 mg to about 1000 mg per day, given as a single once-a-day dose or as divided doses throughout a day.

In one embodiment, the daily dose is administered twice daily in equally divided doses. In certain embodiments, a daily dose range should be from about 10 mg to about 200 mg per day, in other embodiments, between about 10 mg and about 150 mg per day, in further embodiments, between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with patient response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a patient is administered multiple dosages of a composition provided herein, not all of the dosages need be the same. For example, the dosage administered to the patient may be increased to improve the prophylactic or therapeutic effect of the composition or it may be decreased to reduce one or more side effects that a particular patient is experiencing.

In certain embodiment, the dosage of the composition provided herein, based on weight of the active compound, administered to prevent, treat, manage, or ameliorate a disorder, or one or more symptoms thereof in a patient is 0.1 mg/kg, 1 mg/kg, 2 mg/kg, 3 mg/kg, 4 mg/kg, 5 mg/kg, 6 mg/kg, 10 mg/kg, or 15 mg/kg or more of a patient's body weight. In another embodiment, the dosage of the composition or a composition provided herein administered to prevent, treat, manage, or ameliorate a disorder, or one or more symptoms thereof in a patient is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, treatment can be initiated with one or more loading doses of a compound or composition provided herein followed by one or more maintenance doses. In such embodiments, the loading dose can be, for instance, about 60 to about 400 mg per day, or about 100 to about 200 mg per day for one day to five weeks. The loading dose can be followed by one or more maintenance doses. In certain embodiments, each maintenance does is, independently, about from about 10 mg to about 200 mg per day, between about 25 mg and about 150 mg per day, or between about 25 and about 80 mg per day. Maintenance doses can be administered daily and can be administered as single doses, or as divided doses.

In certain embodiments, a dose of a compound or composition provided herein can be administered to achieve a steady-state concentration of the active ingredient in blood or serum of the patient. The steady-state concentration can be determined by measurement according to techniques available to those of skill or can be based on the physical characteristics of the patient such as height, weight and age. In certain embodiments, a sufficient amount of a compound or composition provided herein is administered to achieve a steady-state concentration in blood or serum of the patient of from about 300 to about 4000 ng/mL, from about 400 to about 1600 ng/mL, or from about 600 to about 1200 ng/mL. In some embodiments, loading doses can be administered to achieve steady-state blood or serum concentrations of about 1200 to about 8000 ng/mL, or about 2000 to about 4000 ng/mL for one to five days. In certain embodiments, maintenance doses can be administered to achieve a steady-state concentration in blood or serum of the patient of from about 300 to about 4000 ng/mL, from about 400 to about 1600 ng/mL, or from about 600 to about 1200 ng/mL.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In certain aspects, provided herein are unit dosages comprising a compound, or a pharmaceutically acceptable salt thereof, in a form suitable for administration. Such forms are described in detail above. In certain embodiments, the unit dosage comprises 1 to 1000 mg, 5 to 250 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art.

The dosages of the at least one pharmaceutically active agent for treatment of a viral hepatitis infection are to be used in the combination therapies provided herein. In certain embodiments, dosages lower than those which have been or are currently being used to prevent or treat a viral hepatitis are used in the combination therapies provided herein. The recommended dosages of at least one pharmaceutically active agent for treatment of a viral hepatitis infection can be obtained from the knowledge of those of skill. For those at least one pharmaceutically active agent for treatment of a viral hepatitis infection that are approved for clinical use, recommended dosages are described in, for example, Hardman et al., eds., 1996, Goodman & Gilman's The Pharmacological Basis Of Therapeutics 9th Ed, Mc-Graw-Hill, New York; Physician's Desk Reference (PDR) 57th Ed., 2003, Medical Economics Co., Inc., Montvale, N.J., which are incorporated herein by reference in its entirety.

In various embodiments, the therapeutic substances (e.g., the proteasome inhibitor and the at least one different and optionally the at least one second pharmaceutically active agent for treatment of a viral hepatitis infection) are administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapeutic substances are administered no more than 24 hours apart or no more than 48 hours apart. In certain embodiments, two or more therapies are administered within the same patient visit. In other embodiments, the proteasome inhibitor and the at least one pharmaceutically active agent for treatment of a viral hepatitis infection are administered concurrently.

In other embodiments, the proteasome inhibitor and the at least one pharmaceutically active agent for treatment of a viral hepatitis infection are administered at about 2 to 4 days apart, at about 4 to 6 days apart, at about 1 week part, at about 1 to 2 weeks apart, at about 2 to 3 weeks apart, at about 3 to 4 weeks apart, or more than 4 weeks apart.

Further details on a delay between the administration of the proteasome inhibitor and the pharmaceutically active agent, or agents, for treatment of a viral hepatitis infection, one described elsewhere herein.

In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, a proteasome inhibitor and at least one first and/or second pharmaceutically active agent for treatment of a viral hepatitis infection are administered to a patient, for example, a mammal, such as a human, in a sequence and within a time interval such that the proteasome inhibitor can act together with the other agent(s) to provide an increased benefit than if they were administered otherwise. For example, the at least one first and/or second pharmaceutically active agent can be administered at the same time or sequentially in any order at different points in time; however, if not administered at the same time, they should be administered sufficiently close in time so as to provide the desired therapeutic or prophylactic effect.

In one embodiment, the proteasome inhibitor and the at least one first and/or second pharmaceutically active agent exert their effect at times which overlap. Each pharmaceutically active agent can be administered separately, in any appropriate form and by any suitable route. In other embodiments, the proteasome inhibitor is administered before administration of the pharmaceutically active agent(s).

In certain embodiments, the proteasome inhibitor and the at least one first and/or second pharmaceutically active agent for treatment of a viral hepatitis infection are cyclically administered to a patient. Cycling therapy involves the administration of a first agent (e.g., a first prophylactic or therapeutic agent) for a period of time, followed by the administration of at least one second agent for a period of time and repeating this sequential administration. Cycling therapy can reduce the development of resistance to one or more of the therapies, avoid or reduce the side effects of one of the therapies, and/or improve the efficacy of the treatment.

In certain embodiments, the proteasome inhibitor and the at least one first and/or second pharmaceutically active agent are administered in a cycle of less than about 6 weeks, about once every four weeks, about once every three weeks, about once every two weeks, about once every 10 days or about once every week. One cycle can comprise the administration of a proteasome inhibitor and the at least one first and/or second pharmaceutically active agent for treatment of a viral hepatitis infection by infusion over about 480 minutes every cycle, about 360 minutes every cycle, about 240 minutes every cycle, about 180 minutes every cycle, about 120 minutes every cycle, about 90 minutes every cycle, about 1 hour every cycle, about 45 minutes every cycle, about 30 minutes every cycle, or about 15 minutes every cycle. Each cycle can comprise at least 1 week of rest, at least 2 weeks of rest, at least 3 weeks of rest, or at least 4 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the at least one pharmaceutically active agent for treatment of a viral hepatitis infection are administered separately yet within a time interval such that the proteasome inhibitor can have an additive and/or synergistic effect with the at least one first and/or second pharmaceutically active agent. For example, one component can be administered once per week in combination with the other components that can be administered once every two weeks or once every three weeks. In other words, the dosing regimens are carried out concurrently even if the therapeutics are not administered simultaneously or during the same day.

The at least one first and/or second pharmaceutically active agent for treatment of a viral hepatitis infection can act additively or synergistically with the proteasome inhibitor. In one embodiment, the proteasome inhibitor is administered concurrently with one or more at least one pharmaceutically active agent for treatment of a viral hepatitis infection in the same pharmaceutical composition. In another embodiment, a proteasome inhibitor is administered concurrently with one or more first and/or second pharmaceutically active agents for treatment of a viral hepatitis infection in separate pharmaceutical compositions. In still another embodiment, a proteasome inhibitor is administered prior to administration of one or more first and/or second pharmaceutically active agents for treatment of a viral hepatitis infection. Also contemplated are administration of a proteasome inhibitor and one or more first and/or second pharmaceutically active agents for treatment of a viral hepatitis infection by the same or different routes of administration, e.g., oral and parenteral.

In certain embodiments, when the proteasome inhibitor is administered concurrently with at least one pharmaceutically active agent for treatment of a viral hepatitis infection that potentially produces adverse side effects including, but not limited to, toxicity, the one or more first and/or second pharmaceutically active agents for treatment of a viral hepatitis infection can advantageously be administered at a dose that falls below the threshold that the adverse side effect is elicited.

### Kits

Also provided are kits for use in methods of treatment of a liver disorder, such as virally induced hepatitis, and more specifically HCV infections. The kits can include a proteasome inhibitor, at least one first and/or second pharmaceutically active agent for treatment of a viral hepatitis infection, and optionally instructions providing information to a health care provider regarding usage for treating the disorder. Instructions may be provided in printed form or in the form of an electronic medium such as, e.g., a floppy disc, CD, or DVD, or in the form of a website address where such instructions may be obtained; other re-readable media, e.g., magnetic media, are also envisaged. A unit dose of a proteasome inhibitor, and/or of the at least one first and/or second pharmaceutically active agent for treatment of a viral hepatitis infection, can include a dosage such that when administered to a patient, a therapeutically or prophylactically effective plasma level of the active ingredients(s) can be maintained in the patient for at least 1 day. In some embodiments, a composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a proteasome inhibitor and/or at least one first and/or second pharmaceutically active agent for treatment of a viral hepatitis infection suitable for administration to a patient. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

The invention is illustrated in the following with respect to an example which however is not be construed as being limiting. It will be clear that the scope of claimed subject matter may be practiced otherwise than as particularly described herein. Numerous modifications and variations of the subject matter are possible in view of the teachings herein and, therefore, are within the scope the claimed subject matter.

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### EXAMPLES

### Synthesis of S-2209

The synthesis of S-2209 (S,S,S-[1-[1-[1-Benzyl-2-(2,4-dioxo-imidazolidin-1-ylimino)-ethylcarbamoyl]-2-(1H-indol-3-yl)-ethylcarbamoyl]-2-(1H-indol-3-yl)-ethyl]-carbamic acid benzyl ester) essentially followed the description given in Leban, J., et al., Bioorg. Med. Chem. 2008, 16:4579, specifically scheme 2, page 4583, except fort he following modifications: In step a) 4M HCl in dioxane was used; step b) employed HOBt, HBTU, DIPEA, Cbz-Trp-Trp-OH in EtOAc/DMF at 0-5° C., and step d) was performed in DMF at 0-5° C.

### Clinical Trial

The purpose of this clinical trial was to determine safety and efficacy of PS-341 (bortezomib) in chronic HCV patients. Bortezomib is registered for oncological diseases such as multiple myeloma.

Adult male and female therapy-resistant and therapy refractory patients who had been infected with HCV of genotype 1 and which at the time of the study despite standard therapy showed detectable active HCV replication with high virus titres were included in this clinical study. A further inclusion criterion was that the patients had not received treatment with other HCV medicaments within 28 days prior to treatment commenced. Patients were treated intravenously with bortezomib (Velcade®) at a dosage of 1.3. mg/m² body surface on days 1, 4, 8 and 11. After a final administration of bortezomib, patients were observed for a further 4 weeks.

In summary, no effect, i.e. no significant change of the virus load was determined during treatment with bortezomib. After a therapy-free period, some of the patients reentered standard PEG-IFN plus ribavirin treatment. Surprisingly, 4 to 8 weeks after recommencing therapy with PEG-IFN plus ribavirin, 4 of the 6 patients that had reentered standard therapy were found to be virus-free, e.g., no HCV-RNA was detected in their blood (below the limit of detection of the assay, approx. 10 copies/ml). This observation provided the motivation for performing a detailed follow-up study of these patients.

### Objectives of the Follow-Up Study

The aim of this follow-up study was to assess the course of HCV viral load during standard PEG-IFN and RbV retreatment in patients with chronic HCV after a treatment period with Bortezomib. In the first part of this trial the antiviral efficacy of an 11 day treatment with Bortezomib was tested in patients who had not responded to or tolerated PEG-IFN and RbV therapy. After completion of the first part of this study patients were offered to be retreated with PEG-IFN and highly dosed RbV (13-15 mg/kg body weight) for 72 weeks. Data for this follow-up evaluation were retrospectively captured up to Week 24.

### Study Design

During the first part of the study patients with HCV infection who had not responded to or tolerated PEG-IFN and RbV therapy were treated with Bortezomib (1.3 mg/m² body surface area/dose) for 11 days in a non-randomized, open-label, single-arm design. Patients were followed up for a total of approximately 4 weeks. After completion of the last follow-up visit of the first part (Visit 8), patients were given the option to return to standard therapy with PEG-IFN and RbV for up to 72 weeks. During this period patients received standard care of treatment according to clinical practice and no additional assessments were performed. Data up to Week 24 were retrospectively collected. Patients started standard therapy with PEG-IFN and RbV at Visit 10 (Week 0 of follow-up study) and follow-up data were collected at Week 4 (Visit 11), Week 8 (Visit 12), and Week 12 (Visit 13). Patients with a sufficient viral decline at Week 12, defined as HCV ribonucleic acid (RNA) blood levels ≦100 copies/ml, continued treatment and follow-up data were collected at Week 18 (Visit 14), and Week 24 (Visit 15). Visit 8 data (=last visit of the first part of the study) were used as baseline values for the follow-up study, as patients started to use medication at home without coming to the study center and data planned to be captured at start of treatment (Visit 10) were thus not available.

### Ethics and Patient Information

Prior to inclusion in this observational follow-up study patients were informed about the objectives and procedures of the observation and had to give written informed consent. The addendum for this follow-up study, informed consent documents, and any other appropriate study-related documents were reviewed and approved by the applicable regional independent ethics committee (IEC)

### Assessments

All assessments were done according to local standards of the center. The following data were captured at Visit 11 to Visit 15 (data for Visit 10 were not available, see above):

### Efficacy

HVC viral load;
Lymphocyte count.

### Safety

Adverse events (AEs) and serious AEs (SAES);
Safety laboratory;
Vital signs.
   A schedule of assessments is provided in Table 1.

TABLE 1
Assessment schedule

| | Standard therapy administration | | | | | |
|---|---|---|---|---|---|---|
| | Visit 10¹ | Visit 11 | Visit 12 | Visit 13² | Visit 14³ | Visit 15³ |
| Assessments | Week 0 | Week 4 | Week 8 | Week 12 | Week 18 | Week 24 |
| Informed consent | ✓ | | | | | |
| Therapy before | ✓ | | | | | |
| Bortezomib | | | | | | |
| Therapy after | ✓ | | | | | |
| Bortezomib | | | | | | |
| Hepatitis C viral load | | ✓ | ✓ | ✓ | ✓ | ✓ |
| Lymphocyte counts | | | ✓ | ✓ | ✓ | ✓ |
| Routine safety | | ✓ | ✓ | ✓ | ✓ | ✓ |
| laboratory tests⁴ | | | | | | |
| Vital signs | | ✓ | ✓ | ✓ | ✓ | ✓ |
| Sample retention | | ✓ | ✓ | ✓ | ✓ | ✓ |
| Adverse events | | ✓ | ✓ | ✓ | ✓ | ✓ |

¹Start of re-therapy upon decision of investigator and consent of patient. Patients started to use medication at home without coming to the study center and data planned to be captured at start of treatment (Visit 10) were thus not available
²If positive viral load (>100 copies/ml) was detected at Week 12, therapy was to be stopped and end of study was documented.
³If an increase of viral load was detected (>1 × log10) therapy was stopped and end of study was documented.
⁴Routine laboratory tests included hematology (red and white blood cell count, differential cell count, platelets, hemoglobin, hematocrit), biochemistry (alanine aminotransferase, aspartate aminotransferase, lactate dehydrogenase, glucose, creatinine, creatine kinase, bilirubin), and coagulation (prothrombin time quick test if cirrhosis was confirmed and activated partial thromboplastin time).

### Statistical Methods

No statistical tests were performed and data were generally provided only as patient listings. The course of hepatitis C viral load during the follow-up study was presented graphically. Summary statistics were provided for adverse events (AEs).

### Disposition of Patients

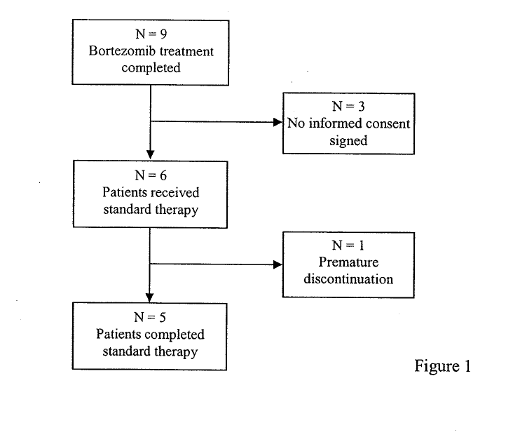

Disposition of patients is shown in FIG. 1. Of the 9 patients who were treated with Bortezomib, 6 patients continued into the follow-up study and received PEG-IFN and RbV standard therapy after the last follow-up visit of the Bortezomib treatment period. One of the 6 patients prematurely discontinued the study (Patient # 106).

### Demographics and Baseline Characteristics

All patients were Caucasian and half of the patients were female. The median age was approximately 50 years.

### History of Standard Therapy

Before Bortezomib treatment in the first part of the study, all patients had received at least 12 weeks of therapy with PEG-IFN and RbV. Patients had received 800 to 1,200 mg per day RbV combined with Pegasys® in one half of the patients and PegIntron® in the other half of patients (Table 2). Patient 103 participated in another clinical study and received a combination of PEG-IFN and RbV and another undisclosed substance. Maximum reduction in virus load had been achieved after 12 to 48 weeks of therapy. A previous relapse was documented for 4 of the 6 patients. All patients presented with a high level of HCV RNA at the start of Bortezomib treatment and at Visit 8 (last visit of the first part of the study).

TABLE 2
Treatment history of study subjects

| | | PEG-IFN | | RbV |
|---|---|---|---|---|
| | Duration | Starting dosage | | Starting dosage |
| Patient | [weeks]^{a} | [μg/week] | Type | [mg/day] |
| 103 | 72 | 180 | Pegasys ® | 1200 |
| 104 | 52 | 100 | PegIntron ® | 1200 |
| 106 | 48 | 180 | Pegasys ® | 1000 |
| 108 | 48 | 80 | PegIntron ® | unknown |
| 109 | 48 | 135^{b} | Pegasys ® | 1200 |
| 110 | 12 | 80 | PegIntron ® | 800 |

^{a}Duration of IFN/RbV treatment prior to entering the clinical trial reported herein
^{b}Dose was increased to 180 μg/week during the course of the therapy.
PEG-IFN = pegylated interferon, RbV = ribavirin.

### Study Performance

### Start of Retreatment

The mean time difference from the last follow-up visit of the Bortezomib treatment period (Visit 8) to the start of standard therapy (Visit 10) was 2.6 weeks; the mean time difference between the last Bortezomib dose and the first dosing event of retreatment was 5.0 weeks (see Table 3, Delay to retreatment). At Visit 10 patients who had agreed to retreatment started retreatment with a combination of RbV (Rebetol® or Copegus®) and PEG-IFN (PegIntron® or Pegasys®) for 72 weeks at doses given in Table 3.

TABLE 3
Dosing of study subjects for retreatment period

| | Delay to | PEG-IFN | | RbV | |
|---|---|---|---|---|---|
| Pa- | retreatment | | Dose | | Dose |
| tient | (weeks) | Type | [μg/week] | Type | [mg/day] |
| 103 | 5.3 | PegIntron ® | 120 | Rebetol ® | 1200 |
| 104 | 4.1 | Pegasys ® | 180 | Rebetol ® | 1400 |
| 106 | 7.6 | PegIntron ® | 150 | Rebetol ® | 1200 |
| 108 | 5.6 | Pegasys ® | 180 | Copegus ® | 1400 |
| 109 | 4.6 | PegIntron ® | 150 | Rebetol ® | 1400 |
| 110 | 3 | Pegasys ® | 180 | Copegus ® | 800 |

PEG-IFN = pegylated interferon, RbV = ribavirin.

### Protocol Deviations

The stopping rule (terminate treatment if HCV RNA >100 copies/ml at Week 12) was not followed in one patient (Patient 106). This patient had a HCV RNA level of 8,900 copies/ml at Week 12 and standard therapy was continued at the patient's request. HCV RNA level in this patient decreased until Visit 15. 2 patients started standard therapy before the intended Visit 10.

### Results

### HCV RNA Values

HCV RNA levels decreased in all patients from Visit 8 to Visit 11 and remained low until Visit 15, where all but one patient (Patient # 106) had HCV RNA levels below 10 copies of viral RNA/ml (FIG. 2). Note that the time intervals between the visits may vary between patients as follow-up visits were performed at the discretion of the investigator. Notably, Patient # 106 was the patient with the longest delay to retreatment (7.6 weeks), suggesting that there may be an upper limit for the time between the end of the proteasome inhibitor treatment and the recommencement of therapy with the pharmaceutical agent for treatment of a viral hepatitis.

### Analysis of Adverse Events

### Relationship to Study Medication

The majority of AEs were assessed as related (possibly, probably, or definitely) to the study treatment. There were 19 AEs which were judged to be definitely related to the study treatment, with fatigue being the most frequent definitely related AE (5 events).

### Severity

The majority of AEs were mild or moderate. There were 2 patients with one severe AE each. Severe AEs included dry mouth and dry skin. Dry mouth was judged to be possibly related to the study medication and resolved without sequelae. Dry skin was judged to be definitely related to the study medication with an outcome of “condition improved”.

### Treatment of Adverse Events

There were 10 AEs which required drug therapy. These AEs were mostly related to “skin and subcutaneous tissue disorders” and “infections and infestations”.

### In Vitro Experiments

The purpose of these experiments is to determine any potential synergistic effects of a double treatment of cells with a proteasome inhibitor (PI) and interferons (IFN) in cell culture.

Combined or sequential treatment of Replicon cells with IFN and PI in cell culture clearly displays synergistic effects. Replicon cells are in general Luc-ubi-neo/ET Replicon cells, which are derived from Huh7 cells and which harbor a sub-genomic HCV Con 1 isolate with a luciferase reporter gene. These so-called Replicon cells allow for in vitro screening of antiviral substances by autonomous HCV RNA replication.

Luc-ubi-neo/ET-cells were seeded and incubated with PI and IFN (Roferon A, Roche) for 24 hours on the following day. All concentrations used for IFN (0.5 U/ml) and PI (500-1000 nM) displayed only a low inhibition of HCV RNA replication in initial experiments. After a 24 hour combined incubation, the medium was changed and cells were incubated for another 48 hours with PI alone. After these 3 days of incubation, HCV RNA replication was determined with respect to the non-treated control. Treatment with PI and IFN showed a reduction of the HCV RNA replication of 30% to a maximum of 50%. Determining HCV RNA replication which had been treated with PI and IFN and subsequently thereto again PI showed only a low basal replication of approximately 10% (i.e. 90% inhibition). This strong inhibition of RNA replication was already observed for very low sub-toxic doses of 750 nM PI and 0.5 U/ml IFN. These concentrations are in a range for which no cytotoxicity is observed.

This representative experiment clearly shows a synergistic effect if HCV-bearing cells are treated with a combination of PI and IFN. These in vitro data support the findings of the above indicated clinical study.

The proteasome inhibitor employed in the experiment described above was S-2209.

Antiviral Activity of Proteasome Inhibitor S-2209 Alone or in Combination with Interferon Alpha (IFN-a) and/or Ribavirin (RbV), Tested in Cell Culture in the HCV Replicon Model System.

### Description of the Huh-7 Luc-ubi-neo/ET Replicon Cells.

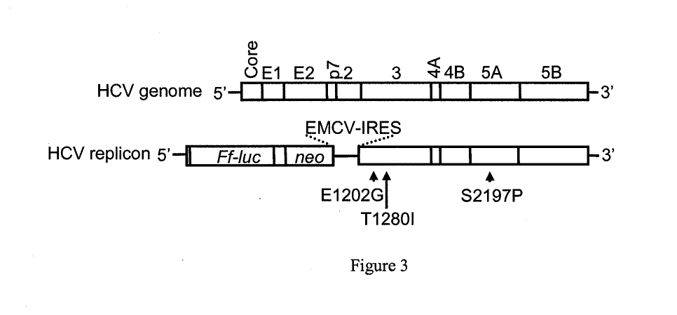

The HCV cell culture system is based on the transfection of the human hepatoma cell line Huh-7 with subgenomic HCV RNAs (Lohmann et al., Replication of subgenomic hepatitis C virus RNAs in a hepatoma cell line. Science 285:110-113 (1999)). These so-called replicons are derived from the full length genome of the HCV Con-1 isolate (EMBL data accession number AJ238799) by deletion of genome-region encoding structural proteins (FIG. 3). In addition, a fusion protein composed of luciferase gene (Ff-luc) from the firefly (Photinus pyralis), as a reporter gene, and the resistance gene neomycin phosphotransferase (neo) was inserted in-between the HCV 5′NTR (non translated region) and NS3-3′NTR region. The translation of neo-luciferase fusion protein is directed by HCV 5′NTR whereas translation of HCV proteins NS3 to NS5B is under the control of the internal ribosome entry site (IRES) of encephalomyocarditis virus (EMCV). In addition, three cell culture-adaptive mutations (E1202G, T1280I, and K1846T), that cooperatively enhance RNA replication in Huh-7 cells, were introduced into the replicon genome. Upon the transfection of Huh-7 cells with corresponding RNAs and G418 selection cells with self-replicating HCV replicons were established (Huh-7 Luc-ubi-neo/ET replicon cells). As described recently the amount of luciferase activity correlates with HCV RNA replication and is therefore a useful in vitro tool for measurement anti-HCV activity of antivirals (Vrolijk et al., A replicon-based bioassay for the measurement of interferons in patients with chronic hepatitis C. J. Virol. Methods 110:201-209 (2003)).

Modeling of Various Treatment Settings to Determine the Most Efficient Application Protocol for Combination-Treatment with S-2209, IFN-a, and Ribavirin.

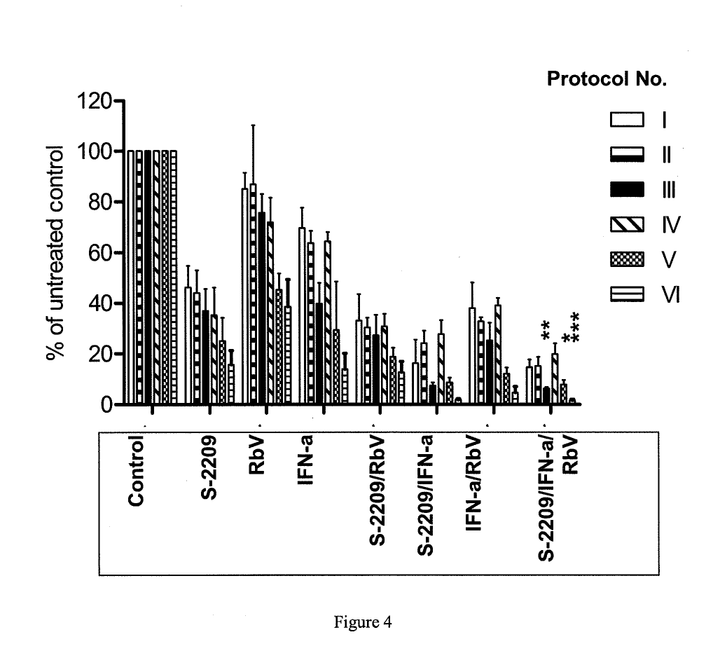

Six different experimental settings were used to monitor the best application procedure of S-2209 in combination with IFN-a (Roferon A, Roche) and Ribavirin (Sigma) (FIG. 4, I-VI). Huh-7 Luc-ubi-neo/ET cells were seeded in 24-well plates at a density of 3-4×10⁴ cells per well, and the respective cell treatment protocol, as described below, was begun 24 h after seeding. For a first experimental protocol (protocol I) cell culture medium of selected wells was replaced by either 1 ml of fresh medium or 1 ml of medium supplemented with 1.85 μM S-2209. Twenty four hours later cell culture medium was removed and cells were cultured for additional 24 h with 1 ml fresh medium or medium either supplemented with 0.75 IU/ml IFN-a, 25 μM Ribavirin or the combination of 0.75 IU/ml IFN-α and 25 μM Ribavirin. A second experimental protocol (protocol II) involved the treatment of cells first with either IFN-a, Ribavirin, or a combination of IFN-a and Ribavirin, at the concentrations given for the first protocol, and 24 h later the cell culture medium was replaced by either 1 ml of fresh medium or 1 ml of medium supplemented with 1.85 μM S-2209. The third protocol (protocol III) mimicked protocol I, with the exception that after the 24 h S-2209-treatment period the cell culture medium was replaced by fresh medium not containing either proteasome inhibitor, IFN-a or Ribavirin, and only after a further 24 h incubation cells were treated with IFN-a and/or Ribavirin. Experimental protocols IV, V and VI involved a single, double, or triple, respectively, 24 h treatment of cells with either medium only, or medium containing either S-2209, IFN-a, Ribavirin or any of the permutations possible by combining these three substances, at the concentrations given for protocol I above. For all protocols, the antiviral effect was determined 24 h after the last treatment by comparing the results of a luciferase assay as described below.

### Determination of Antiviral Effect.

Quantification of luciferase reporter activity was used to determine the antiviral effects in Huh-7 Luc-ubi-neo/ET replicon cells. Cells, treated in duplicate for each individual drug-combination, were harvested at the given time. To this end, cells were washed once with PBS, 250 μl of lysis buffer (0.1% Triton X-100, 10% Glycerol, 25 mM glycylglycine, 15 mM MgSO4, 4 mM EGTA and 1 mM DTT, pH 7.8) was added and freeze-thaw lysates were prepared. For each well, two times 100 μA lysate was mixed with 100 μl assay buffer (10% Glycerol, 25 mM glycylglycine, 15 mM MgSO4, 4 mM EGTA, 1 mM DTT, 2 mM ATP and 15 mM K2PO4, pH 7.8) and, after addition of 40 μl of a luciferin solution (1 mM luciferin, 25 mM glycylglycine, pH 7.8), measured for 2 s in a luminometer (Synergy 2 HT, Biotek). The antiviral effect was determined by normalizing the relative light units (RLU) of the different applications to the corresponding values obtained with untreated cells. Means and standard deviations of three independent experiments are shown. Statistical analysis of cells treated with S-2209, IFN-a, and Ribavirin was performed by using one-tailed t-test, comparing individual experimental protocols versus protocol I. P levels <0.05 were considered as statistically significant (FIG. 4, rightmost panel, *p≦0.02, **p≦0.005, ***p≦0.001).

### Results

The results obtained from the luciferase assays under protocols I to VI are summarized in FIG. 4. Treating Huh-7 cells with proteasome inhibitor first and IFN-a/RbV subsequently (protocol I) had an effect on replication that was indistinguishable from the effect of the reverse treatment (protocol II). Allowing an additional 24 h to elapse after proteasome inhibitor treatment but before IFNa or IFN-a/RbV treatment under protocol III had a markedly enhanced effect in lowering viral propagation. The most pronounced effect was obtained with a triple treatment with proteasome inhibitor and IFNa or IFN-a/RbV.

1. A kit for the treatment of a hepatitis viral infection in a human or animal who does not respond or is refractory to treatment with at least one pharmaceutically active agent for treatment of a viral hepatitis infection, the kit comprising:
(a) first pharmaceutical container containing a composition comprising an effective amount of a proteasome inhibitor;
(b) a second pharmaceutical container containing a composition comprising an effective amount of a first pharmaceutically active agent for treatment of a viral hepatitis infection; and
(c) instructions in paper or electronic form advising a user to apply the kit for the treatment of a human or animal individual who does not respond or is refractory to treatment with at least one pharmaceutically active agent for treatment of a viral hepatitis infection.

2. The kit according to claim 1, wherein said second container further contains a composition comprising an effective amount of a second pharmaceutically active agent for treatment of a viral hepatitis infection which is different from said first pharmaceutically active agent for treatment of a viral hepatitis infection.

3. The kit according to claim 1, further comprising a third pharmaceutical container containing a composition comprising an effective amount of a second pharmaceutically active agent for treatment of a viral hepatitis infection which is different from said first pharmaceutically active agent for treatment of a viral hepatitis infection.

4. The kit according to claim 2 or 3, wherein:
(a) said proteasome inhibitor is a proteasome-specific inhibitor;
(b) said first pharmaceutically active agent for treatment of a viral hepatitis infection is an agent which supports or assists a human or animal body's natural response in dealing with a viral infection; and
(c) said second pharmaceutically active agent for treatment of a viral hepatitis infection is an agent which interferes with the function of a viral target.

5. The kit according to claim 2 or 3, wherein:
(a) said proteasome inhibitor is a proteasome-specific inhibitor;
(b) said first pharmaceutically active agent for treatment of a viral hepatitis infection is an interferon or a derivative thereof, an interleukin, a steroid, an immunomodulator, an immunosuppressant or an inhibitor of assisted protein folding; and
(c) said second pharmaceutically active agent for treatment of a viral hepatitis infection is an inhibitor of HCV HCV NS3/4A protease, HCV NS4B protein, HCV NS5A protein, HCV NS5B polymerase or of an HCV envelope protein.

6. The kit according to claim 2 or 3, wherein:
(a) said protease inhibitor is a protease-specific inhibitor;
(b) said first pharmaceutically active agent for treatment of a viral hepatitis infection is an interferon or a derivative thereof; and
(c) said second pharmaceutically active agent for treatment of a viral hepatitis infection is a nucleoside analog or a nucleotide analog.

7. The kit according to claim 2 or 3, wherein:
(a) said first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040, or a glycosylated, pegylated or hesylated form thereof; and
(b) said second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 AND ACH-806 (GS-9132).

8. The kit according to claim 2 or 3, wherein:
(a) said first pharmaceutically active agent for treatment of a viral hepatitis infection is a pegylated form of interferon-alpha, beta, gamma or omega; and
(b) said second pharmaceutically active agent for treatment of a viral hepatitis infection is ribavirin.

9. The kit according to claim 1, wherein said proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins (−)-epigallocatechin-3-gallate, Disulfuram, acridine derivatives, tetra-acridine derivatives with betulinic acid, as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides, TMC-95A, TMC-95A endocyclic oxindole-phenyl clamp (BIA-1a) derivatives, TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

10. The kit according to claim 9, wherein said proteasome inhibitor is PS-273, PS-341, PS-519 or S-2209.

11. The kit according to claim 7, wherein said proteasome inhibitor is PS-341, said first pharmaceutically active agent for treatment of a viral hepatitis infection is pegylated interferon alpha, and said second pharmaceutically active agent for treatment of a viral hepatitis infection is ribavirin.

12. The kit according to claim 9, wherein said proteasome inhibitor is S-2209.

13. The kit according to claim 9, wherein said proteasome inhibitor is S-2209, said first pharmaceutically active agent for treatment of a viral hepatitis infection is pegylated interferon alpha, and said second pharmaceutically active agent for treatment of a viral hepatitis infection is ribavirin.

14. The kit according claim 1, wherein said instructions advise said user to administer the compositions of said first and said second pharmaceutical containers concurrently.

15. The kit according to claim 1, wherein said instructions advise said user to first administer said composition of said first pharmaceutical container and administer said composition of said second pharmaceutical container subsequently after a delay.

16. The kit according to claim 1, wherein said instructions advise said user to first administer said composition of said second pharmaceutical container and administer said composition of said first pharmaceutical container subsequently after a delay.

17. The kit according to claim 15 or 16, wherein said instructions further advise the user of the length of said delay.

18. The kit according to claim 17, wherein said delay is from about 2 weeks to about 8 weeks.

19. The kit according to claim 1, wherein said instructions advise said user to perform one to two rounds of administrations, each round consisting of 3 to 10 administrations of said composition of said first pharmaceutical container.

20. The kit according claim 1, wherein said instructions advise said user to administer said first pharmaceutically active agent for treatment of a viral hepatitis infection for not more than about 50% of the duration otherwise recommended for the treatment of viral hepatitis infection with said first pharmaceutically active agent and/or to administer not more than about 66% of the dose recommended for the treatment of hepatitis viral infection with said first pharmaceutically active agent for treatment of a viral hepatitis infection.

21. The kit according to claim 1, wherein said instructions advise said user to apply the kit for the treatment of a human or animal individual ineligible for or unwilling to undergo treatment with a pharmaceutically active agent for treatment of a viral hepatitis infection.

22. The kit according to claim 1, wherein the hepatitis viral infection is an infection with Hepatitis C virus.

23. A method for treating a hepatitis viral infection in a human or animal individual who does not respond or is refractory to treatment with a pharmaceutically active agent for treatment of viral hepatitis infection, the method comprising administering to said human or animal in need thereof an effective amount of a proteasome inhibitor and an effective amount of a first pharmaceutically active agent for treatment of a viral hepatitis infection.

24. The method of claim 23, further comprising administering to said human or animal an effective amount of a second pharmaceutically active agent for treatment of a viral hepatitis infection which is different from said first pharmaceutically active agent.

25. The method of claim 23, wherein:
(a) said proteasome inhibitor is a proteasome-specific inhibitor; and
(b) said first pharmaceutically active agent for treatment of a viral hepatitis infection is an agent which supports or assists a human or animal body's natural response to a viral infection or an agent that interferes with the function of a viral target.

26. The method of claim 24, wherein:
(a) said proteasome inhibitor is a proteasome-specific inhibitor;
(b) said first pharmaceutically active agent for treatment of a viral hepatitis infection is an agent which support or assists the human or animal body's natural response to a viral infection; and
(c) said second pharmaceutically active agent for treatment of a viral hepatitis infection is an agent that interferes with the function of a viral target.

27. The method of claim 23, wherein
(a) said proteasome inhibitor is a proteasome-specific inhibitor; and
(b) said first pharmaceutically active agent for treatment of a viral hepatitis infection is an interferon or a derivative thereof, an interleukin, a steroid, an immunomodulator, an immunosuppressant, an inhibitor of assisted protein folding, an inhibitor of HCV HCV NS3/4A protease, an inhibitor of HCV NS4B protein, an inhibitor of HCV NS5A protein, an inhibitor of HCV NS5B polymerase or an inhibitor of an HCV envelope protein.

28. The method of claim 24, wherein
(a) said proteasome inhibitor is a proteasome-specific inhibitor;
(b) said first pharmaceutically active agent for treatment of a viral hepatitis infection is an interferon or a derivative thereof, an interleukin, a steroid, an immunomodulator, an immunosuppressant or an inhibitor of assisted protein folding; and
(c) said second pharmaceutically active agent for treatment of a viral hepatitis infection is an inhibitor of HCV HCV NS3/4A protease, HCV NS4B protein, HCV NS5A protein, HCV NS5B polymerase or of an HCV envelope protein.

29. The method of claim 23 or 24, wherein said first pharmaceutically active agent for treatment of a viral hepatitis infection is an interferon or a derivative thereof, a nucleoside analog or a nucleotide analog.

30. The method of claim 24, wherein
(a) said first pharmaceutically active agent for treatment of a viral hepatitis infection is an interferon or a derivative thereof; and
(b) said second pharmaceutically active agent for treatment of a viral hepatitis infection is a nucleoside analog or a nucleotide analog.

31. The method of claim 23 or 24, wherein said first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon, Belerofon, Pegasys, Peg-IFN lambda, PEGIntron, ANA773, SD101, IMO-2125, GI-5005 (Tarmogen), IC41, PF-04878691, TG 4040 or a glycosylated, pegylated or hesylated form thereof, lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

32. The method of claim 24, wherein
(a) said first pharmaceutically active agent for treatment of a viral hepatitis infection is interferon-alpha, beta, gamma or omega, albinterferon, Locteron, Omega IFN, Medusa IFN, DA-3021, EMZ702, Infradure, IL-28, IL-29, Veldona, Soluferon and Belerofon or a glycosylated, pegylated or hesylated form thereof; and
(b) said second pharmaceutically active agent for treatment of a viral hepatitis infection is lamivudine, cidovir, ribavirin, viramidine, didanosine, vidarabine, cytarabine, emtricitabine, zalcitabine, abacavir, stavudine, zidovudine, idoxuridine, trifluridine, valopiticabine, R1626, R7128, IDX184, HCV-796, Filibuvir (PF 00868554), VCH-916, ANA598, BI 207127, VCH-222 PSI-6130, MK-3281, ABT-072, ABT-333, R1728, VCH-759, GS9190, BMS-650032, BE-868554, Debio-025, NIM-811, SCY-635, PPI-461, PPI-1301, AZD7295, EDP-239, IDX-NS5A, AZD2836, BMS-790052, Alinia (nitazoxanide), BMS-791325, BMS-824393, Celgosivir, BILB 1941, IDX-375, PSI-7851, PSI-7977 (single isomer of PSI-7851), BI201335, ABT-450, ACH-1625, AVL-181, BILN-2061, Boceprevir (SCH503034), GS-9256, IDX-320, ITMN-191 (RG7227, RO5190591), ITMN-5489, MK7009, TMC435 (TMC435350), VX-813, VX-985, ACH-1095, A-831, KPE02001003, TCM700C, PYN-17, BIT225, JTK-652, BMS-791325 or ACH-806 (GS-9132).

33. The method of claim 23 or 24, wherein said first pharmaceutically active agent for treatment of a viral hepatitis infection is a pegylated form of interferon-alpha, beta, gamma or omega, or is ribavirin.

34. The method of claim 24, wherein
(a) said first pharmaceutically active agent for treatment of a viral hepatitis infection is a pegylated form of interferon-alpha, beta, gamma or omega; and
(b) said second pharmaceutically active agent for treatment of a viral hepatitis infection is ribavirin.

35. The method of claim 23 or 24, wherein said proteasome inhibitor is selected from the group consisting of peptides carrying at their C-terminal, α,β-epoxyketone, vinyl-sulphones, glyoxal or boronic acid-residues, pinacol-esters; chemically modified derivatives of naturally occurring proteasome inhibitors, epoxomycine, carfilzomib, eponemycine, aclacinomycine A (aclarubicine), celastrol, withaferin A, Gliotoxin, epipolythiodioxo-piperazines, green tea polyphenolic catechins, (−)-epigallocatechin-3-gallate, Disulfuram, acridine derivatives, tetra-acridine derivatives with betulinic acid, 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides such as TMC-95A, TMV-95A endocyclic oxindole-phenyl clamp (BIA-1a) TMC-95A endocyclic biphenyl-ether clamp (BIA-2a) derivatives, lactacystine, Omuralide, Homobelactosin C, Salinosporamide A, NEOSH-101, CEP-18770, IPSI001, IPSI007, MLN2238, MLN9708, ONX 0912, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)₂, pyrazyl-2,5-bis-CONH(CHPhe)CONH(CHisobutyl)-B(OH)₂, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S,4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)₂ and its enantiomer PS-293, 8-quinolyl-sulfonyl-CONH—(CH-napthyl)-CONH(—CH-isobutyl)-B(OH)₂; NH₂(CH-Napthyl)-CONH—(CH-isobutyl)-B(OH)₂; morpholino-CONH—(CH-napthyl)-CONH—(CH-phenylalanine)-B(OH)₂; CH₃—NH—(CH-napthyl-CONH—(CH-isobutyl)-B(OH)₂; 2-quinole-CONH—(CH-homo-phenylalanin)-CONH—(CH-isobutyl)-B(OH)₂; Phenyalanine-CH₂—CH₂—CONH—(CH-phenylalanine)-CONH—(CH-isobutyl)1-B(OH)₂; “PS-383” (pyridyl-CONH—(CHpF-phenylalanine)-CONH—(CH-isobutyl)-B(OH)₂, (PEG)₁₉₋₂₅-Leu-Leu-Nle-H, (PEG)₁₉₋₂₅-Arg-Val-Arg-H, H-Nle-Leu-Leu-(PEG)₁₉₋₂₅-Leu-Leu-Nle-H, H-Arg-Val-Arg-(PEG)₁₉₋₂₅-Arg-Val-Arg-H ZLLL-vs), ZLLVS, YLVS, MG-262, ALLnL, ALLnM, LLnV, DFLB Ada-(Ahx)₃-(Leu)₃-vs; YU101 (Ac-hFLFL-ex), MLN519, S-2209, Compound 1-6 and Compound 8.

36. The method of claim 35, wherein said proteasome inhibitor is PS-273, PS-341, PS-519 or S-2209.

37. The method of claim 24, wherein said proteasome inhibitor is PS-341, said first pharmaceutically active agent for treatment of a viral hepatitis infection is pegylated interferon alpha, and said second pharmaceutically active agent for treatment of a viral hepatitis infection nucleoside analog is ribavirin.

38. The method of claim 36, wherein said proteasome inhibitor is S-2209.

39. The method of claim 24, wherein said proteasome inhibitor is S-2209, wherein said first pharmaceutically active agent for treatment of a viral hepatitis infection is pegylated interferon alpha, and wherein said second pharmaceutically active agent for treatment of a viral hepatitis infection is ribavirin.

40. The method of claim 23, wherein said proteasome inhibitor and said first pharmaceutically active agent for treatment of a viral hepatitis infection are administered concurrently.

41. The method of claim 24, wherein said proteasome inhibitor, said first pharmaceutically active agent for treatment of a viral hepatitis infection and said second pharmaceutically active agent for treatment of a viral hepatitis infection are all administered concurrently.

42. The method of claim 23, wherein said proteasome inhibitor is administered first and said first pharmaceutically active agent for treatment of a viral hepatitis infection is administered subsequently after a delay.

43. The method of claim 24, wherein said proteasome inhibitor is administered first and said first pharmaceutically active agent for treatment of a viral hepatitis infection and said second pharmaceutically active agent for treatment of a viral hepatitis infection are administered subsequently after a delay.

44. The method of claim 23, wherein said first pharmaceutically active agent for treatment of a viral hepatitis infection is administered first and said proteasome inhibitor is administered subsequently after a delay.

45. The method of claim 24, wherein said first pharmaceutically active agent for treatment of a viral hepatitis infection and the second pharmaceutically active agent for treatment of a viral hepatitis infection are administered first, and said proteasome inhibitor is administered subsequently after a delay.

46. The method of any one of claims 42-45, wherein said delay is from about 2 weeks to about 8 weeks.

47. The method of claim 23, wherein said proteasome inhibitor is administered in one to two rounds of administrations, wherein each round of administration includes 3 to 10 administrations.

48. The method of claim 24, wherein said first pharmaceutically active agent for treatment of a viral hepatitis infection is administered for not more than about 50% of the duration otherwise recommended for the treatment of the hepatitis viral infection with said first pharmaceutically active agent and/or such that not more than about 66% of the dose recommended for the treatment of the viral hepatitis infection with said first pharmaceutically active agent is administered.

49. The method of claim 24, wherein said first and/or said second pharmaceutically active agent for treatment of a viral hepatitis infection is administered for not more than about 50% of the duration otherwise recommended for the treatment of the hepatitis viral infection with the respective pharmaceutically active agent for treatment of a viral hepatitis infection and/or such that not more than about 66% of the dose recommended for the treatment of the viral hepatitis infection with the respective pharmaceutically active agent is administered.

50. The method of claim 48 or 49, wherein said human or animal is ineligible for or unwilling to undergo treatment with a pharmaceutically active agent for treatment of a viral hepatitis infection.

51. The method of claim 23 or 24, wherein the hepatitis viral infection is an infection with Hepatitis C virus.

52. A pharmaceutical composition comprising: an effective amount of PS-341 or S-2209; and an effective amount of a pegylated interferon-alpha.

53. The pharmaceutical composition of claim 52, further comprising an effective amount of ribavirin.

54. A kit for the treatment of a hepatitis viral infection in a human or animal who does not respond or is refractory to treatment with at least one pharmaceutically active agent for treatment of a viral hepatitis infection, the kit comprising:
(a) first pharmaceutical container containing a composition comprising an effective amount of a proteasome inhibitor;
(b) a second pharmaceutical container containing a composition comprising an effective amount of a first pharmaceutically active agent for treatment of a viral hepatitis infection, wherein said first pharmaceutically active agent for treatment of a viral hepatitis infection is an agent which supports or assists a human or animal body's natural response in dealing with a viral infection or interferes with the function of a viral target; and
(c) instructions in paper or electronic form advising a user to apply the kit for the treatment of a human or animal individual who does not respond or is refractory to treatment with at least one pharmaceutically active agent for treatment of a viral hepatitis infection.

55. The kit according to claim 54, wherein said second container further contains a composition comprising an effective amount of a second pharmaceutically active agent such that one of said first or said second pharmaceutically active agent assists a human or animal body's natural response in dealing with a viral infection, while the other of said first or second pharmaceutically active agents interferes with the function of a viral target, wherein said first and said second pharmaceutically active agent are both different from said proteasome inhibitor.

56. The kit according to claim 54, further comprising a third pharmaceutical container containing a composition comprising an effective amount of a second pharmaceutically active agent such that one of said first or said second pharmaceutically active agent assists a human or animal body's natural response in dealing with a viral infection, while the other of said first or second pharmaceutically active agents interferes with the function of a viral target, wherein said first and said second pharmaceutically active agent are both different from said proteasome inhibitor.

57. A method for treating a hepatitis viral infection in a human or animal individual who does not respond or is refractory to treatment with a pharmaceutically active agent for treatment of viral hepatitis infection, the method comprising administering to said human or animal in need thereof an effective amount of a proteasome inhibitor and an effective amount of a first pharmaceutically active agent for treatment of a viral hepatitis infection, wherein said first pharmaceutically active agent for treatment of a viral hepatitis infection is an agent which supports or assists a human or animal body's natural response in dealing with a viral infection or interferes with the function of a viral target.

58. The method of claim 57, further comprising administering to said human or animal an effective amount of a second pharmaceutically active agent for treatment of a viral hepatitis infection such that one of said first or said second pharmaceutically active agent assists a human or animal body's natural response in dealing with a viral infection, while the other of said first or second pharmaceutically active agents interferes with the function of a viral target, wherein said first and said second pharmaceutically active agent are both different from said proteasome inhibitor.
